SEQUENCE LISTING (37 sequences)

SEQ ID NO: 1 - protein, 525 aa, Aspergillus fumigatus
MRTAAASLTLAATCLFELASALMPRAPLIPAMKAKVALPSGNATFEQYIDHNNPGLGTFPQRYWYNPEFWAGPGSPVLLFTPGESDAADYDGFLTNKTIVGRFAEEIGGAVILLEHRYWGASSPYPELTTETLQYLTLEQSIADLVHFAKTVNLPFDEIHSSNADNAPWVMTGGSYSGALAAWTASIAPGTFWAYHASSAPVQAIYDFWQYFVPVVEGMPKNCSKDLNRVVEYIDHVYESGDIERQQEIKEMFGLGALKHFDDFAAAITNGPWLWQDMNFVSGYSRFYKFCDAVENVTPGAKSVPGPEGVGLEKALQGYASWFNSTYLPGSCAEYKYWTDKDAVDCYDSYETNSPIYTDKAVNNTSNKQWTWFLCNEPLFYWQDGAPKDESTIVSRIVSAEYWQRQCHAYFPEVNGYTFGSANGKTAEDVNKWTKGWDLTNTTRLIWANGQFDPWRDASVSSKTRPGGPLQSTEQAPVHVIPGGFHCSDQWLVYGEANAGVQKVIDEEVAQIKAWVAEYPKYRKP

SEQ ID NO: 2 - protein, 644 aa, Aspergillus fumigatus
MRLSHVLLGTAAAAGVLASPTPNDYVVHERRAVLPRSWTEEKRLDKASILPMRIGLTQSNLDRGHDLLMEISDPRSSRYGQHLSVEEVHSLFAPSQETVDRVRAWLESEGIAGDRISQSSNEQFLQFDASAAEVERLLGTEYYLYTHQGSGKSHIACREYHVPHSLQRHIDYITPGIKLLEVEGVKKARSIEKRSFRSPLPPILERLTLPLSELLGNTLLCDVAITPLCISALYNITRGSKATKGNELGIFEDLGDVYSQEDLNLFFSTFAQQIPQGTHPILKAVDGAQAPTSVTNAGPESDLDFQISYPIIWPQNSILFQTDDPNYTANYNFSGFLNTFLDAIDGSYCSEISPLDPPYPNPADGGYKGQLQCGVYQPPKVLSISYGGAEADLPIAYQRRQCAEWMKLGLQGVSVVVASGDSGVEGRNGDPTPTECLGTEGKVFAPDFPATCPYLTTVGGTYLPLGADPRKDEEVAVTSFPSGGGFSNIYERADYQQQAVEDYFSRADPGYPFYESVDNSSFAENGGIYNRIGRAYPDVAAIADNVVIFNKGMPTLIGGTSAAAPVFAAILTRINEERLAVGKSTVGFVNPVLYAHPEVFNDITQGSNPGCGMQGFSAATGWDPVTGLGTPNYPALLDLFMSLP

SEQ ID NO: 3 - protein, 602 aa, Aspergillus fumigatus
MFSSLLNRGALLAVVSLLSSSVAAEVFEKLSAVPQGWKYSHTPSDRDPIRLQIALKQHDVEGFETALLEMSDPYHPNYGKHFQTHEEMKRMLLPTQEAVESVRGWLESAGISDIEEDADWIKFRTTVGVANDLLDADFKWYVNEVGHVERLRTLAYSLPQSVASHVNMVQPTTRFGQIKPNRATMRGRPVQVDADILSAAVQAGDTSTCDQVITPQCLKDLYNIGDYKADPNGGSKVAFASFLEEYARYDDLAKFEEKLAPYAIGQNFSVIQYNGGLNDQNSASDSGEANLDLQYIVGVSSPIPVTEFSTGGRGLLIPDLSQPDPNDNSNEPYLEFLQNVLKMDQDKLPQVISTSYGEDEQTIPEKYARSVCNLYAQLGSRGVSVIFSSGDSGVGAACLTNDGTNRTHFPPQFPAACPWVTSVGGTTKTQPEEAVYFSSGGFSDLWERPSWQDSAVKRYLKKLGPRYKGLYNPKGRAFPDVAAQAENYAVFDKGVLHQFDGTSCSAPAFSAIVALLNDARLRAHKPVMGFLNPWLYSKASKGFNDIVKGGSKGCDGRNRFGGTPNGSPVVPYASWNATDGWDPATGLGTPDFGKLLSLAMRR

SEQ ID NO: 4 - protein, 596 aa, Aspergillus fumigatus
MAPFTFLVGILSLCICCIVLGAAAEPSYAVVEQLRNVPDGWIKHDAAPASELIRFRLAMNQERAAEFERRVIDMSTPGHSSYGQHMKRDDVREFLRPPEEVSDKVLSWLRSENVPAGSIESHGNWVTFTVPVSQAERMLRTRFYAFQHVETSTTQVRTLAYSVPHDVHRYIQMIQPTTRFGQPARHERQPLFHGTVATKEELAANCSTTITPNCLRELYGIYDTRAEPDPRNRLGVSGFLDQYARYDDFENFMRLYATSRTDVNFTVVSINDGLNLQDSSLSSTEASLDVQYAYSLAYKALGTYYTTGGRGPVVPEEGQDTNVSTNEPYLDQLHYLLDLPDEELPAVLSTSYGEDEQSVPESYSNATCNLFAQLGARGVSIIFSSGDSGVGSTCITNDGTKTTRFLPVFPASCPFVTAVGGTHDIQPEKAISFSSGGFSDHFPRPSYQDSSVQGYLEQLGSRWNGLYNPSGRGFPDVAAQATNFVVIDHGQTLRVGGTSASAPVFAAIVSRLNAARLEDGLLKLGFLNPWLYSLNQTGFTDIIDGGSSGCYVGTSNEQLVPNASWNATPGWDPVTGLGTPIYNTLVKLATSVSSTP

SEQ ID NO: 5 - protein, 594 aa, Aspergillus fumigatus
MLSSTLYAGWLLSLAAPALCVVQEKLSAVPSGWTLIEDASESDTITLSIALARQNLDQLESKLTTLATPGNPEYGKWLDQSDIESLFPTASDDAVLQWLKAAGITQVSRQGSLVNFATTVGTANKLFDTKFSYYRNGASQKLRTTQYSIPDHLTESIDLIAPTVFFGKEQNSALSSHAVKLPALPRRAATNSSCANLITPDCLVEMYNLGDYKPDASSGSRVGFGSFLNESANYADLAAYEQLFNIPPQNFSVELINRGVNDQNWATASLGEANLDVELIVAVSHPLPVVEFITGGSPPFVPNADEPTAADNQNEPYLQYYEYLLSKPNSHLPQVISNSYGDDEQTVPEYYARRVCNLIGLMGLRGITVLESSGDTGIGSACMSNDGTNKPQFTPTFPGTCPFITAVGGTQSYAPEVAWDGSSGGFSNYFSRPWYQSFAVDNYLNNHITKDTKKYYSQYTNFKGRGFPDVSAHSLTPYYEVVLTGKHYKSGGTSAASPVFAGIVGLLNDARLRAGKSTLGFLNPLLYSILAEGFTDITAGSSIGCNGINPQTGKPVPGGGIIPYAHWNATAGWDPVTGLGVPDFMKLKELVLSL

SEQ ID NO: 6 - protein, 395 aa, Aspergillus fumigatus
MVVFSKVTAVVVGLSTIVSAVPVVQPRKGFTINQVARPVTNKKTVNLPAVYANALTKYGGTVPDSVKAAASSGSAVTTPEQYDSEYLTPVKVGGTTLNLDFDTGSADLWVFSSELSASQSSGHAIYKPSANAQKLNGYTWKIQYGDGSSASGDVYKDTVTVGGVTAQSQAVEAASHISSQFVQDKDNDGLLGLAFSSINTVSPRPQTTFFDTVKSQLDSPLFAVTLKYHAPGTYDFGYIDNSKFQGELTYTDVDSSQGFWMFTADGYGVGNGAPNSNSISGIADTGTTLLLLDDSVVADYYRQVSGAKNSNQYGGYVFPCSTKLPSFTTVIGGYNAVVPGEYINYAPVTDGSSTCYGGIQSNSGLGFSIFGDIFLKSQYVVFDSQGPRLGFAPQA

SEQ ID NO: 7 - protein, 269 aa, Aspergillus fumigatus
MKFTSVLASGLLATAAIAAPLTEQRQARHARRLARTANRSSHPPYKPGTSEVIKLSNTTQVEYSSNWAGAVLIGTGYTAVTGEFVVPTPSVPSGGSSSKQYCASAWVGIDGDTCSSAILQTGVDFCIQGSSVSFDAWYEWYPDYAYDFSGISISAGDTIRVTVDATSKTAGTATVENVTKGKTVTHTFTGGVDGNLCEYNAEWIVEDFESNGSLVPFANFGTVTFTGAQATDGGSTVGPSGATLIDIQQSGKVLTSVSTSSSSVTVKYV

SEQ ID NO: 8 - protein, 613 aa, Aspergillus fumigatus
MLSLVTLLSGTAGLALTASAQYFPPTPEGLKVVHSKHQEGVKISYKEPGICETTPGVKSYSGYVHLPPGTLNDVDVDQQYPINTFFCFFESRNDPIHAPLAIWMNGGPGSSSMIGLLQENGPCLVNADSNSTEINPWSWNNYVNMLYIDQPNQVGFSYDVPTNGTYNQLTTAWNVSAFPDGKVPEQNNTFYVGTFPSMNRTATANTTQNAARSLWHFAQTWFSEFPEYKPHDDRVSIWTESYGGRYGPSFAAFFQEQNEKIEEGALPDEYHYIHLDTLGIINGCVDLLTQAPFYPDMAYNNTYGIEAINKTVYERAMNAWSKPGGCKDLIVKCRELAAEGDPTMSGHNETVNEACRRANDYCSNQVEGPYILFSKRGYYDIAHFDPDPFPPPYFQGFLNQNWVQAALGVPVNFSISVDSTYSAFASTGDYPRADVHGYLEDLAYVLDSGIKVALVYGDRDYACPWNGGEEVSLRVNYSDSQSFQKAGYAPVQTNSSYIGGRVRQYGNFSFTRVFEAGHEVPAYQPQTAYEIFHRALFNRDIATGKMSLLKNATYASEGPSSTWEFKNEVPESPEPTCYIQSLQSSCTEEQIQSVVNGTALIKDWIVVEKVDIY

SEQ ID NO: 9 - protein, 765 aa, Aspergillus fumigatus
MKWSILLLVGCAAAIDVPRQPYAPTGSGKKRLTFNETVVKRAISPSAISVEWISTSEDGDYVYQDQDGSLKIQSIVTNHTQTLVPADKVPEDAYSYWIHPNLSSVLWATNYTKQYRHSYFADYFIQDVQSMKLRPLAPDQSGDIQYAQWTPTGDAIAFVRDNNVFVWTNASTSQITNDGGPDLFNGVPDWIYEEEILGDRFALWFSPDGAYLAFLRFNETGVPTFTVPYYMDNEEIAPPYPRELELRYPKVSQTNPTVELNLLELRTGERTPVPIDAFDAKELIIGEVAWLTGKHDVVAVKAFNRVQDRQKVVAVDVASLRSKTISERDGTDGWLDNLLSMAYIGPIGESKEEYYIDISDQSGWAHLWLFPVAGGEPIALTKGEWEVTNILSIDKPRQLVYFLSTKHHSTERHLYSVSWKTKEITPLVDDTVPAVWSASFSSQGGYYILSYRGPDVPYQDLYAINSTAPLRTITSNAAVLNALKEYTLPNITYFELALPSGETLNVMQRLPVKFSPKKKYPVLFTPYGGPGAQEVSKAWQALDFKAYIASDPELEYITWTVDNRGTGYKGRAFRCQVASRLGELEAADQVFAAQQAAKLPYVDAQHIAIWGWSYGGYLTGKVIETDSGAFSLGVQTAPVSDWRFYDSMYTERYMKTLESNAAGYNASAIRKVAGYKNVRGGVLIQHGTGDDNVHFQNAAALVDTLVGAGVTPEKLQVQWFTDSDHGIRYHGGNVFLYRQLSKRLYEEKKRKEKGEAHQWSKKSVL

SEQ ID NO: 10 - DNA, 1578 bp, Aspergillus fumigatus
atgcggactgctgctgcttcactgacgcttgctgcgacttgtctctttgagttggcatctgctctcatgcccagggcgcctttgatccctgcgatgaaagcgaaagttgccttgccctctggaaacgcgacattcgagcagtatattgatcataataaccccggtctgggaacatttccccagagatactggtataatccggagttttgggccggtcctggctctcctgtgcttttgtttacaccgggtgaatcagatgctgcggactacgacggattcctgaccaacaagacgattgttggacgctttgccgaagagatcgggggcgcggttatcctgcttgagcatcgctactggggagcctcatcaccttatcccgagttgaccaccgagacgctccagtacctgactctggagcagtcgatcgcagaccttgttcactttgcaaagactgtgaatcttccgttcgacgagattcacagcagcaacgccgataacgcgccatgggtgatgactgggggatcctacagtggtgctctagccgcgtggaccgcatcaattgctccagggaccttctgggcgtaccatgcatcgagtgcaccggtgcaggccatctatgacttctggcaatatttcgtccccgttgtcgaggggatgcccaagaactgcagcaaggatctcaaccgcgtggtggagtatattgaccacgtctatgagtcgggggatatcgagcgccagcaggaaatcaaagagatgttcgggttgggagctctcaagcattttgacgattttgcagcagcaattacgaacggaccatggctttggcaggatatgaatttcgtctcggggtactcccgtttttataaattttgcgatgcggtagagaatgtcactccgggggcaaagtccgttcctggaccggaaggcgtcggtctggagaaagcactccaaggctatgcgtcatggttcaattcaacgtacttgcctggctcttgcgccgaatacaaatattggaccgacaaagacgcagttgactgttacgactcttatgagactaacagccccatttacaccgacaaggccgtcaacaatacctccaataagcagtggacctggttcttatgcaatgaacctctcttctactggcaagatggtgcacccaaggatgagtccaccattgtctccagaatcgtctcagcagagtactggcagcgacaatgtcacgcgtatttcccagaagtcaacggctatacgttcggtagcgccaatggcaagaccgctgaagacgtgaataagtggaccaagggctgggacttgaccaacacaacacgtctgatctgggcaaatggtcaattcgatccctggagggacgcctcagtttcctccaaaacgagacccggaggaccccttcagtccacagaacaagcgccagtacatgtaattccgggtgggttccattgctcagatcaatggctagtctatggggaggcgaatgccggcgttcaaaaggtgattgatgaagaagtggcgcaaatcaaggcttgggtcgcggagtatcccaaatataggaagccatga

SEQ ID NO: 11 - DNA, 1935 bp, Aspergillus fumigatus
atgcgactttcacacgtactcctaggaactgcagctgcagctggcgttctggctagtcccaccccgaacgactatgtcgtgcatgaacgtcgtgctgtcctccctcgctcctggacggaggagaagagacttgataaggcctctatcttgcctatgaggattggtctcactcagtctaacctagatcgcggtcatgacttgttgatggagatatctgatccgcgctcgtcacgctatggacaacatctctccgtcgaggaggtccacagtctctttgctccgagccaggagactgtcgaccgtgttcgagcatggcttgagtctgagggcatagccggcgaccgcatctctcagtcctcgaacgagcaattcctgcaatttgacgcgagtgcggcggaagttgaaaggctattgggtactgagtactatctctatacacatcaaggttcaggaaagtcacacattgcttgccgagaataccatgtcccccactcattgcagcggcatatcgactacattacccctggcatcaagctcctagaggtggaaggagtcaagaaagctcggagcattgaaaagcgttcattcagaagcccgctgccgccaatccttgagcggcttacccttcccttgtccgagctgctgggtaatactttattgtgtgatgtggccataacaccactgtgtatatcagctctctacaacattactcgcggctcaaaagctaccaagggcaatgaactgggcatctttgaggatctaggggatgtttacagtcaagaggatctcaacctgttcttttcaacatttgcacagcaaattccccagggcactcatcccatcctgaaggccgtcgacggcgctcaagccccaaccagcgtgaccaatgcagggcccgaatccgacctggactttcaaatctcgtatccgatcatctggccgcagaactccattctctttcaaacagatgatccaaattacacagcaaactacaacttcagtggctttttgaacacctttttggatgctatcgatggatcctactgcagcgagatctcccctctggacccgccgtaccccaatcccgccgacggcggctacaaaggccaactccagtgcggcgtctaccagccccccaaggttctctccatctcgtacggcggcgccgaggccgacctccccatcgcgtaccagcgccgccagtgcgccgagtggatgaaactcggcctgcagggtgtctccgtcgtcgtcgcatccggcgactccggcgtcgaaggcaggaatggcgatcccacccccactgagtgcctcgggacggaagggaaagtcttcgccccggacttcccggccacctgtccctacctcaccaccgtcggcgggacctacctccccctcggcgccgacccccgcaaggacgaagaagtcgccgtgacctcgttcccctcgggcggcgggttcagcaacatctacgagcgcgcagactaccagcagcaagccgtcgaggactacttctcccgcgccgatcccgggtacccgttctacgagagcgtcgacaacagcagcttcgcggagaacggcggcatctacaaccggattgggcgcgcgtacccggacgtcgcagccatcgcggacaacgtcgtgatcttcaacaagggcatgccgacgcttattggcggtacctcggctgctgcgccggtgtttgcagccatcctgactaggattaacgaggagcggctcgcggtcggcaagtcgaccgtgggatttgtgaaccccgtgctgtatgcgcatcccgaggtgtttaatgatatcacgcaggggagtaacccgggctgtggcatgcaagggttctccgctgcgacgggatgggatccggtgacggggttgggaactccgaattatccagcacttttagacttgttcatgagcctgccgtag

SEQ ID NO: 12 - DNA, 1809 bp, Aspergillus fumigatus
atgttttcgtcgctcttgaaccgtggagctttgctcgcggttgtttctctcttgtcctcttccgttgctgccgaggtttttgagaagctgtccgcggtgccacagggatggaaatactcccacacccctagtgaccgcgatcccattcgcctccagattgccctgaagcaacatgatgtcgaaggttttgagaccgccctcctggaaatgtccgatccctaccacccaaactatggcaagcactttcaaactcacgaggagatgaagcggatgctgctgcccacccaggaggcggtcgagtccgtccgcggctggctggagtccgctggaatctcggatatcgaggaggatgcagactggatcaagttccgcacaaccgttggcgtggccaatgacctgctggacgccgacttcaagtggtacgtgaacgaggtgggccacgttgagcgcctgaggaccctggcatactcgctcccgcagtcggtcgcgtcgcacgtcaacatggtccagcccaccacgcggttcggacagatcaagcccaaccgggcgaccatgcgcggtcggcccgtgcaggtggatgcggacatcctgtccgcggccgttcaagccggcgacacctccacttgcgatcaggtcatcacccctcagtgcctcaaggatctgtacaatatcggcgactacaaggccgaccccaacgggggcagcaaggtcgcgtttgccagtttcctggaggaatacgcccgctacgacgatctggccaagttcgaggagaagctggccccgtacgccattggacagaactttagcgtgatccagtacaacggcggtctgaacgaccagaactccgccagtgacagcggggaggccaatctcgacctgcagtacatcgttggtgtcagctcgcccattccggtcaccgagttcagcaccggtggccggggtcttctcattccggacctgagccagcccgaccccaacgacaacagcaacgagccgtatctggaattcctgcagaatgtgttgaagatggaccaggataagctccctcaggtcatctccacctcctatggcgaggatgaacagaccattcccgaaaaatacgcgcgctcggtctgcaacctgtacgctcagctgggcagccgcggggtttcggtcattttctcctctggtgactccggtgttggcgcggcttgcttgaccaacgacggcaccaaccgcacgcacttccccccacagttccctgcggcctgcccctgggtgacctcggtgggtggcacgaccaagacccagcccgaggaggcggtgtacttttcgtcgggcggtttctccgacctgtgggagcgcccttcctggcaggattcggcggtcaagcgctatctcaagaagctgggccctcggtacaagggcctgtacaaccccaagggccgtgccttccccgatgttgctgcccaggccgagaactacgccgtgttcgacaagggggtgctgcaccagtttgacggaacctcgtgctcggctcccgcatttagcgctatcgtcgcattgctgaacgatgcgcgtctgcgcgctcacaagcccgtcatgggtttcctgaacccctggctgtatagcaaggccagcaagggtttcaacgatatcgtcaagggcggtagcaagggctgcgacggtcgcaaccgattcggaggtactcccaatggcagccctgtggtgccctatgccagctggaatgccactgacggctgggacccggccacgggtctagggactccggactttggcaagcttctgtctcttgctatgcggagatag

SEQ ID NO: 13 - DNA, 1791 bp, Aspergillus fumigatus
atggctccattcacgtttctggtagggatactatccctctgtatttgctgcattgttcttggtgcagctgcagagcccagctacgcggtcgttgagcagctcagaaatgttcccgacggctggataaagcacgatgcagcgccagcgtctgaattgatcagatttcggctggctatgaaccaggaaagagccgctgaattcgagcgaagggtcattgacatgtcaacgccgggtcactcgagctatggacaacatatgaagcgtgacgatgtcagggaatttctgcgtcctcccgaggaggtttcagacaaagtcctttcctggctgagatcagagaatgttcctgctggctcgattgaaagtcatggcaactgggtcactttcactgtcccggtatcacaggcggaacgtatgctaagaacacgcttttacgccttccagcacgtggagacaagtacgacacaagtcagaacgcttgcgtattccgttccacatgacgtccaccgctatattcagatgatccagccaacgactcgctttggacaacctgcccggcatgaacggcaaccacttttccacgggactgttgctaccaaggaagagctggcggcgaattgctccacaaccataacgccgaactgccttcgcgaattgtacgggatttatgataccagagccgaacccgatccccgcaacagactgggagtttccgggttcctagatcagtacgcacgttacgacgactttgaaaattttatgagattgtatgcaaccagtaggacagacgtcaacttcactgtggtctcgataaatgacggtctcaatctgcaggactcgtccctgagcagtaccgaagccagcctagacgtccagtatgcctattctttggcgtataaagcgcttggaacctactatacaacgggtggccgaggaccggttgtgcctgaggaaggtcaggatacgaacgtgtcgaccaatgagccttacttagatcaacttcattatcttcttgatcttccagatgaagagcttcccgccgttctttcaacctcgtatggtgaagatgagcaaagcgtccctgaatcatactcaaatgcaacatgcaatctgttcgcgcagcttggcgcacgcggcgtgtcgatcatcttcagcagcggtgactcaggcgttggttcaacatgcataactaacgatggaaccaagacaactcgattcttgcctgtcttcccagcgtcctgcccatttgttactgctgtcggcggtactcacgatatccaacccgagaaagcaattagcttctctagcggaggcttttcagatcactttccacgtccctcctatcaggattcaagcgttcaaggctacctagagcagcttggaagcagatggaacgggttatacaacccgagcgggagaggtttccctgacgtcgccgctcaggccactaactttgtcgtcattgatcacgggcaaacgttgagggtaggcggcacaagtgcatctgcgcctgtatttgcagccatagtctcgcgattaaatgctgctcgacttgaggatggtttgctaaaactggggttcttaaatccatggctctattccctcaaccagacaggattcacagacattattgatggtggctcatcgggttgctatgttggcaccagcaacgagcaactggttcccaatgcaagctggaatgcaacgccaggatgggatcctgttaccgggcttgggacgcccatttataataccctggtgaaattggccacgagtgtttcaagtaccccatga

SEQ ID NO: 14 - DNA, 1707 bp, Aspergillus fumigatus
atgctgtcctcgactctctacgcagggtggctcctctccctcgcagccccagccctttgtgtggtgcaggagaagctctcagctgttcctagtggctggacactcatcgaggatgcatcggagagcgacacgatcactctctcaattgcccttgctcggcagaacctcgaccagcttgagtccaagctgaccacgctggcgaccccagggaacccggagtacggcaagtggctggaccagtccgacattgagtccctatttcctactgcaagcgatgatgctgttctccaatggctcaaggcggccgggattacccaagtgtctcgtcagggcagcttggtgaacttcgccaccactgtgggaacagcgaacaagctctttgacaccaagttctcttactaccgcaatggtgcttcccagaaactgcgtaccacgcagtactccatccccgatcacctgacagagtcgatcgatctgattgcccccactgtcttctttggcaaggagcagaacagcgcactgtcatctcacgcagtgaagcttccagctcttcctaggagggcagccaccaacagttcttgcgccaacctgatcacccccgactgcctagtggagatgtacaacctcggcgactacaaacctgatgcatcttcgggaagtcgagtcggcttcggtagcttcttgaatgagtcggccaactatgcagatttggctgcgtatgagcaactcttcaacatcccaccccagaatttctcagtcgaattgatcaacagaggcgtcaatgatcagaattgggccactgcttccctcggcgaggccaatctggacgtggagttgattgtagccgtcagccaccccctgccagtagtggagtttatcactggcgccctacctccagtactacgagtacttgctctccaaacccaactcccatcttcctcaggtgatttccaactcactgttcccgagtactacgccaggagagtttgcaacttgatcggcttgatgggtcttcgtggcatcacggtgctcgagtcctctggtgataccggaatcggctcggcatgcatgtccaatgacggcaccaacaagccccaattcactcctacattccctggcacctgccccttcatcaccgcagttggtggtactcagtcctatgctcctgaagttgcttgggacggcagttccggcggattcagcaactacttcagccgtccctggtaccagtctttcgcggtggacaactacctcaacaaccacattaccaaggataccaagaagtactattcgcagtacaccaacttcaagggccgtggattccctgatgtttccgcccatagtttgaccccttactacgaggtcgtcttgactggcaaacactacaagtctggcggcacatccgccgccagccccgtctttgccggtattgtcggtctgctgaacgacgcccgtctgcgcgccggcaagtccactcttggcttcctgaacccattgctgtatagcatcctggccgaaggattcaccgatatcactgccggaagttcaatcggttgtaatggtatcaacccacagaccggaaagccagttcctggtggtggtattatcccctacgctcactggaacgctactgccggctgggatcctgttactggccttggggttcctgatttcatgaaattgaaggagttggttctgtcgttgtaa

SEQ ID NO: 15 - DNA, 1188 bp, Aspergillus fumigatus
atggtcgtctttagcaaagtcaccgctgtcgtcgtcggtctctcgaccattgtgtctgctgtccctgtggtccagccgcgcaagggcttcactatcaaccaagtggccagaccagtgaccaacaagaagaccgtcaatcttccagctgtctatgccaatgctttgactaagtacgggggcactgtccccgacagtgtcaaggcggctgcaagctccggcagcgctgttactacccccgagcaatatgactcggaatacctgacccccgtcaaagtcggtggaacgaccctgaacttggacttcgacactggctctgcagatctctgggtcttctcctccgagctttcggcttcccagtccagcggccatgctatctacaagccgtccgctaatgcccaaaagctgaatggctacacctggaagatccaatatggtgatggtagcagtgccagcggtgacgtctacaaggataccgtcactgtgggtggtgtcactgctcagagccaggctgtggaggctgccagccatatcagctctcaattcgtgcaggataaggacaacgatggtctgttgggtttggcattcagctccatcaacactgtcagtccccgccctcagactactttctttgacactgtcaagtcccagttggactctcctctctttgctgtgaccttgaagtaccatgctccaggcacctacgactttggatacatcgacaactccaagttccaaggggaactcacttataccgacgtcgacagctcccagggtttctggatgttcactgctgatggctacggtgttggcaatggtgctcccaactccaacagtatcagcggcattgctgacaccggcaccaccctcctcctgcttgatgacagcgttgttgccgactactaccgccaggtttccggagccaagaacagcaaccaatacggtggttatgtcttcccctgctccaccaaacttccttctttcactaccgtcatcggaggctacaatgccgtcgttcccggtgaatacatcaactacgcccccgtcactgacggcagctctacctgctacggcggcatccagagcaactctggtttgggcttttctatcttcggagatatcttcctcaagagccagtacgtcgtcttcgactcccaaggccccagactcggcttcgcccctcaggcatag

SEQ ID NO: 16 - DNA, 810 bp, Aspergillus fumigatus
atgaagttcacttctgtcctcgcctccggcttgcttgccacggctgccatcgctgctcccctcacagaacagcgtcaagcccggcatgcccgtcgtctggcccgcaccgccaacagatcgagccaccctccctacaagcccggcacttccgaggttatcaagctcagcaacaccacccaggtcgagtacagctccaactgggctggtgccgtcctcatcggcacaggctacacggctgtgactggcgagttcgtcgtccctacccccagcgtcccaagcggtggctcttccagcaagcagtactgcgcctccgcttgggtcggtatcgacggtgacacctgcagctctgccatcctgcaaaccggcgtcgacttctgcatccagggcagctctgtctccttcgacgcctggtacgagtggtaccccgactacgcgtacgacttcagcggcatctccatctccgctggcgacacgatcagggtcaccgttgatgcaaccagcaagaccgctggcacggccactgtcgagaatgtgaccaagggcaagactgtcacccacaccttcaccggcggcgtggacggcaatctgtgcgagtacaatgccgagtggatcgttgaagactttgagtccaacgggtctctggtgccgtttgctaactttggcactgtcaccttcaccggggctcaggctaccgatggcggttccactgttgggccttctggcgccactctgattgatatccagcagagcggcaaggttttgacttcggtttctacctctagcagctctgtcactgttaagtatgtctaa

SEQ ID NO: 17 - DNA, 1842 bp, Aspergillus fumigatus
atgctatccctcgtaacccttctatctgggaccgctggtcttgcattgaccgcgtcggcacagtatttccctcccactcccgagggtctcaaggtcgtgcattcgaagcaccaggagggcgtgaagatttcgtacaaagaacctggtatttgtgaaaccaccccgggtgtcaaatcgtactccggctatgtacatctgccgcccggcacgctgaacgacgttgatgtcgaccagcaataccccatcaacactttcttctgcttcttcgagtcgcgcaatgatcccattcacgcaccgctggccatttggatgaacggcggtcccggcagctcgtccatgatcggactactgcaggaaaatggcccgtgtcttgtaaacgccgactccaactcaacggagatcaacccctggtcgtggaacaactacgtcaacatgctgtacattgatcagccgaaccaggttgggttcagctacgatgttcctacaaacgggacgtataaccagctcaccactgcgtggaatgtgtctgcattcccggatggtaaagtcccggagcagaacaatacattctatgtgggcacgttccccagtatgaaccggacggctacggcaaatacgacgcagaatgcggcgcggtcgctttggcactttgcgcagacgtggttctctgaattccccgagtacaagccgcacgatgaccgggtgagtatctggactgagtcatatggtggtcgatacgggccgtcgttcgcggcgttctttcaggaacagaatgagaagatcgaagagggggcgttaccagatgagtaccattacattcacctggacactctgggaatcatcaatgggtgcgtggatttgttgacccaagcgccgttctacccggatatggcgtacaacaatacctacggcatcgaggcgatcaacaagaccgtctacgaaagggcaatgaatgcgtggagtaagcccggtggctgcaaggacctgatagtcaagtgccgtgagctagcggccgagggagatccaaccatgtccggccacaacgagacggtcaacgaggcctgtcgaagggcgaacgactactgcagcaaccaggtggaaggcccctacatactgttctccaagcgtggctactacgatatcgcgcactttgatccagatccatttccaccaccttatttccaaggtttcctgaaccagaactgggtacaagccgccctgggggtgcccgtcaacttctccatctcagtggacagcacatacagcgcctttgcgtcgacgggcgactatccgcgcgccgatgttcacgggtacctcgaggatcttgcatatgtcctcgactcggggatcaaagtggcgctcgtctacggagaccgggactacgcatgtccctggaacggcggcgaagaggttagtttgcgcgtcaactattccgactcgcagtcgttccaaaaagcaggctacgccccggtccagaccaattcgtcatatatcgggggccgggtgcggcagtacggcaacttttctttcacgcgtgtcttcgaagcgggccatgaggtgccagcgtatcaaccgcagacggcctatgagatcttccacagagcgttatttaatcgagacattgcgacggggaagatgtcactactgaagaatgccacctacgcgagcgagggcccatcctcgacgtgggaatttaagaatgaggtacctgagagtccggagccgacctgttatatccagtcattgcagagtagttgcaccgaagagcagatccagagcgtggtcaacggcactgctttgattaaagattggatcgtggtggagaaagtggacatttactag

SEQ ID NO: 18 - DNA, 2298 bp, Aspergillus fumigatus
atgaagtggtcaattctccttttggtcggctgcgctgccgccattgacgtccctcgtcaaccatatgcccctactggaagcggcaagaaacgactgaccttcaacgagacggtcgtcaagcgagccatttccccctcggccatctcggtcgagtggatttctacctccgaggatggggattatgtctaccaagaccaggacggcagtctgaaaatccagagcatcgtcaccaaccacacgcagaccctcgtccctgcggacaaagtgccagaggatgcctacagctactggatccatcccaatctctcctccgtgctctgggctaccaactacaccaagcaataccggcactcgtactttgccgactactttatccaggacgtgcagtcgatgaaattgcgaccgctcgccccagaccagtccggcgacatccagtacgctcagtggactcccaccggcgacgccatcgcctttgtccgcgacaacaacgtcttcgtctggaccaatgcctcgactagccagattaccaatgacggcgggccggatctcttcaatggcgtcccggactggatctacgaggaggagatcctcggcgaccggtttgcgctctggttctcgccggacggggcgtacctcgccttcctgcggttcaatgagaccggtgtcccaaccttcaccgtgccgtactacatggacaacgaggagattgcgccgccgtacccacgcgagctggagctgcggtatcccaaggtgtcgcagacgaaccctaccgtcgagctgaacctgctggagctccgtaccggcgagcggacgcctgtcccgatcgacgcctttgacgcaaaggagctgatcatcggcgaggtggcgtggttgacggggaagcatgacgtcgtggctgtcaaggcgttcaaccgcgtgcaggaccggcaaaaggtcgtcgctgtggatgtggcctcgctcaggtccaagacaattagtgagcgcgacggcacggacggatggctggataacctgctctccatggcgtacatcgggcccatcggcgagtccaaggaggagtactacattgacatctcggaccagtccggctgggcgcatctctggctgtttcctgtcgccggaggcgagcccatcgccctgaccaagggcgagtgggaagtcaccaatatccttagcatcgacaagccgcgccagctggtctacttcctgtcgaccaaacaccacagcaccgagcgccacctctactccgtctcctggaagacgaaagaaatcacccccttagtcgacgacaccgtccccgccgtctggtccgcctccttctcctcgcagggcggatactacatcctctcttaccgcgggcccgacgtgccctaccaagacctctacgccatcaactccaccgcgcccctgcgcaccatcaccagcaacgcggccgtgctcaacgccttgaaggaatacaccttgccgaacattacctacttcgagctcgcccttcccagcggcgaaaccctcaacgtcatgcagcgcctccccgtcaagttctcccccaagaagaagtaccccgttctcttcaccccctacggcggtcccggcgcacaagaagtctccaaagcctggcaagccctcgacttcaaggcctacattgcctcagaccccgaactcgagtatatcacctggacggttgacaaccgcggcacgggctacaagggccgcgcattccggtgccaagttgccagccggctgggcgagctcgaagccgccgaccaggtcttcgccgcgcagcaggccgccaagctcccttatgtcgacgcacagcacatcgccatatggggatggagttacggcggctatctgacgggcaaggtcatcgagaccgacagtggggcgttctcgcttggtgtgcagaccgctccggtttcggactggcgattctatgattcgatgtacacggagcggtatatgaagacgctggagagcaacgcggcagggtacaatgccagtgcgatccggaaggtagcaggctacaagaatgtgcgtggtggggtgctgatccagcatgggacgggtgacgataatgtgcatttccagaatgcggcggcgctggtggacacccttgttggggcgggagtgacaccggagaagctgcaggtgcagtggtttacagactcggatcatgggattcggtaccatggggggaatgtgttcttgtatcggcagttgtccaagaggctgtacgaggagaagaagcggaaggagaagggtgaggcgcatcagtggagcaagaagtctgttctgtag

SEQ ID NO: 19 - DNA, 31 bp, Artificial
  synthetic sequence
gtttctcgagcactcatgcccagggcgcctt

SEQ ID NO: 20 - DNA, 25 bp, Artificial
  synthetic sequence
tgagagctcccaacccgaacatctc

SEQ ID NO: 21 - DNA, 24 bp, Artificial
  synthetic sequence
tgggagctctcaagcattttgact

SEQ ID NO: 22 - DNA, 30 bp, Artificial
  synthetic sequence
gtttagatctcatggcttcctatatttggg

SEQ ID NO: 23 - DNA, 48 bp, Artificial
  synthetic sequence
gtttagatctcagtgatggtgatggtgatgtggcttcctatatttggg

SEQ ID NO: 24 - DNA, 31 bp, Artificial
  synthetic sequence
gttccatgggtggctttggcaggatatgaat

SEQ ID NO: 25 - DNA, 30 bp, Artificial
  synthetic sequence
cttggatcctcatggcttcctatatttggg

SEQ ID NO: 26 - protein, 36 aa, Artificial
  synthetic sequence
YPSKPDNPGEDAPAEDMARYYSALRHYINLITRQRY

SEQ ID NO: 27 - protein, 34 aa, Artificial
  synthetic sequence
SKPDNPGEDAPAEDMARYYSALRHYINLITRQRY

SEQ ID NO: 28 - protein, 9 aa, Artificial
  synthetic sequence
RPPGFSPFR

SEQ ID NO: 29 - protein, 31 aa, Artificial
  synthetic sequence
DNPGEDAPAEDMARYYSALRHYINLITRQRY

SEQ ID NO: 30 - protein, 28 aa, Artificial
  synthetic sequence
GEDAPAEDMARYYSALRHYINLITRQRY

SEQ ID NO: 31 - protein, 23 aa, Artificial
  synthetic sequence
AEDMARYYSALRHYINLITRQRY

SEQ ID NO: 32 - protein, 8 aa, Artificial
  synthetic sequence
YPSKPDNP

SEQ ID NO: 33 - protein, 6 aa, Artificial
  synthetic sequence
SKPDNP

SEQ ID NO: 34 - protein, 5 aa, Artificial
  synthetic sequence
GEDAP

SEQ ID NO: 35 - protein, 6 aa, Artificial
  synthetic sequence
GFSPFR

SEQ ID NO: 36 - protein, 7 aa, Artificial
  synthetic sequence
PGFSPFR

SEQ ID NO: 37 - protein, 7 aa, Artificial
  synthetic sequence
RPPGFSP

(19) United States
(12) Patent Application Publication
(10) Pub. No.: US 2012/0276075 A1
(43) Pub. Date: Nov. 1, 2012

(54) SYNERGIC ACTION OF A PROLYL PROTEASE AND TRIPEPTIDYL PROTEASES

(75) Inventors: Michel Monod, Lausanne (CH); Eric Grouzmann, Lausanne (CH)

(73) Assignee: CENTRE HOSPITALIER UNIVERSITAIRE VAUDOIS (CHUV), Lausanne (CH)

(21) Appl. No.: 13/517,141

(22) PCT Filed: Dec. 20, 2010
(86) PCT No.: PCT/IB10/55959

§ 371 Date: Jul. 16, 2012

(30) Foreign Application Priority Data
Dec. 21, 2009 (IB) PCT/IB2009/055884

(51) Int. Cl. C12N 9/62; C07K 1/12; A62D 3/02; C12Q 1/37; A61K 38/48; A61K 38/54; A61P 1/00; A61P 37/08; A61P 31/10; A61K 8/66; A01N 63/02; A01P 1/00; A23L 1/105; A23J 3/34; A23C 19/032; C12N 9/48

(52) U.S. Cl. 424/94.2; 435/225; 435/212; 435/272; 435/262.5; 435/23; 424/94.64; 426/18; 426/7; 426/36

(57) ABSTRACT

The present invention relates to a novel enzyme composition comprising a prolyl protease and tripeptidyl proteases having unique catalytic properties. The present invention further relates to methods for producing the enzyme composition as well as a pharmaceutical composition and a food supplement containing the enzyme composition and its use in the degradation of polypeptides.

### FIELD OF THE INVENTION

The present invention relates to a novel enzyme composition comprising a prolyl protease and tripeptidyl proteases having unique catalytic properties. The present invention further relates to methods for producing the enzyme composition as well as a pharmaceutical composition and a food supplement containing the enzyme composition and its use in the degradation of polypeptides.

### BACKGROUND OF THE INVENTION

Celiac disease (CD) is a digestive genetically determined disorder that damages the small intestine and interferes with absorption of nutrients from food. People who have CD cannot tolerate a protein called gluten, which is found in wheat, rye and barley. The disease has a prevalence of about 1:200 in most of the world's population groups and the only treatment for CD is to maintain a life-long, strictly gluten-free diet. For most people, following this diet will stop symptoms, heal existing intestinal lesions, and prevent further damage. The disease is more frequent in the paediatric population. Patients are suspected of having CD when they are presenting gastrointestinal or malabsorption symptoms. The principal toxic components of wheat gluten are a family of proline- and glutamine-rich proteins called gliadins, which are resistant to degradation in the gastrointestinal tract and contain several T-cell stimulatory epitopes (33 mer and 31-49 (p31-49) peptides). The 33-mer peptide is an excellent substrate for the enzyme transglutaminase 2 (TG2) that deamidates the immunogenic gliadin peptides, increasing their affinity to human leucocyte antigen (HLA) DQ2 or DQ8 molecules and thus activating the T cell-mediated mucosal immune response leading to clinical symptoms. The toxicity of these fragments may be due to an overexpression of transferrin receptor in CD allowing intestinal transport of intact peptide across the enterocyte. Thus the peptides can escape degradation by the acidic endosome-lysosomal pathway only in patients with active CD and can reach the serosal border unchanged.

Since in patients with coeliac disease the gastrointestinal tract does not possess the enzymatic equipment to efficiently cleave the gluten-derived proline-rich peptides, driving the abnormal immune intestinal response, another therapeutic approach relies on the use of orally active proteases to degrade toxic gliadin peptides before they reach the mucosa. Oral therapy by exogenous prolyl-endopeptidases able to digest ingested gluten was therefore propounded as an alternative treatment to the diet.

It has been demonstrated (Shan et al., Science 2002) that an exogenous PEP (prolyl endoprotease) derived from Flavobacterium meningosepticum helps to digest gliadin peptides. The addition of PEP either in vitro in the presence of brush border membrane (BBM) extracts or during in vivo perfusion of rat small intestine caused a rapid degradation of the 33 mer peptide and a loss of its capacity to stimulate gliadin-specific T cells.

A randomized, double-blind, cross-over study in twenty asymptomatic patients with histologically proven celiac sprue involving two 14-day stages has been performed using gluten pretreated with recombinant PEP from F. meningosepticum. The result of this study was not very satisfactory mainly because PEP from F. meningosepticum exhibits pH optima near neutrality and is not active in the stomach.

To circumvent this problem, PEP was associated to a glutamine-specific endoprotease B, iso form 2 from Hordeum vulgare (EP-B2), a cysteine-protease derived from germinating barley seeds that is activated at acidic pH and by pepsin and can efficiently hydrolyse gliadin in vitro in conditions mimicking the gastric lumen (Bethune et al., Chem. Biol., 2006). Another study proved that the combination of EP-B2 with PEP from F. meningosepticum improve the breakdown of gluten. Also another reports that a PEP deriving from Aspergillus niger, deploying its main activity under acid conditions in the stomach, can start to degrade gliadin before it reached the intestinal lumen. (Stepniak et al., Am J. Physiol. Gastrointest. Liver Physiol., 2006).

WO2005019251 (Funzyme Biotechnologies SA) provides leucine aminopeptidase (LAP) of two different fungal species, Trichophyton rubrum and Aspergillus fumigatus in combination with dipeptidyl peptidase IV (DppIV). These enzymes have been evaluated for cleavage of the 33 mer under neutral pH condition since the optimal activity of LAPs were estimated around 7.0 with a range of activity between pH 6 and 8. However, a limitation of these enzymes relies on their optimum activity at neutral pH precluding a possible breakdown of gliadin in the gastric fluid.

Another known oral therapy by exogenous peptidases is the use of encapsulated undefined enzyme extract, such as Combizym® containing the combination of digestive enzymes of pancreatin (lipase, amylase, protease) and enzyme concentrate from Aspergillus oryzae containing protease, cellulase, hemicellulase, and amylase.

The problem to be solved to confer a potential therapeutic value to an enzyme or enzyme composition are the following: the enzymes must be resistant to degradation by other gastrointestinal enzymes, efficient in the environment where the 33 mer is produced, must present a high proteolytic activity toward gluten peptides, should be active at acidic pH and should be able to access a complex composition of gluten hindered by other components of normal foodstuffs eventually baked or cooked.

The Applicants were able to solve this problem in the present invention by providing an enzyme composition having unique catalytic properties.

### SUMMARY OF THE INVENTION

The Applicants provide in the present invention an improved enzyme composition, comprising
i. a prolyl protease AfuS28 comprising SEQ ID NO: 1, a biologically active fragment thereof, a naturally occurring allelic variant thereof, or a sequence having at least 95% of identity, and
ii. at least one tripeptidyl protease of the sedolisin family, said tripeptidyl protease selected from the group consisting in
   a) a sedolisin SedA comprising SEQ ID NO: 2, a biologically active fragment thereof, a naturally occurring allelic variant thereof, or a sequence having at least 95% of identity, or
   b) a sedolisin SedB comprising SEQ ID NO: 3, a biologically active fragment thereof, a naturally occurring allelic variant thereof, or a sequence having at least 95% of identity, or
   c) a sedolisin SedC comprising SEQ ID NO: 4, a biologically active fragment thereof, a naturally occurring allelic variant thereof, or a sequence having at least 95% of identity, or
   d) a sedolisin SedD comprising SEQ ID NO: 5, a biologically active fragment thereof, a naturally occurring allelic variant thereof, or a sequence having at least 95% of identity

The invention further relates to a pharmaceutical composition comprising an enzyme composition of the invention and at least one pharmaceutically acceptable excipient, carrier and/or diluent.

Additionally, the invention relates to a food supplement comprising an enzyme composition of the invention.

The invention also encompasses an enzyme composition for use in a method for treating and/or preventing a syndrome associated with a human disease, said disease being selected from the group comprising celiac disease, digestive tract bad absorption, an allergic reaction, an enzyme deficiency, a fungal infection, Crohn disease, mycoses, wound healing and sprue.

Additionally, the invention encompasses the use of an enzyme composition for the degradation of proteins, for the degradation of by-products, toxic or contaminant proteins; for the degradation of prions or viruses; for the degradation of proteins for proteomics; for the degradation of cornified substrate; for the hydrolysis of polypeptides for amino acid analysis; for wound cleaning; for cosmetology such as peeling tools, depilation, dermabrasion and dermaplaning; for prothesis cleaning and/or preparation; for fabric softeners; for soaps; for tenderizing meat; for the controlled fermentation process of Soja or cheese; for cleaning or disinfection of septic tanks or any container containing proteins that should be removed or sterilized; and for cleaning of surgical instruments.

The invention also provides a method of degrading a polypeptide substrate comprising contacting the polypeptide substrate with an enzyme composition of the invention.

Further, the invention provides a method of detoxifying gliadin comprising contacting gliadin containing food product with an effective dose of an enzyme composition of the invention.

Additionally, the invention concerns a method for improving food digestion in a mammal comprising oral administration to the said mammal of an enzyme composition of the invention.

The invention also involves a kit for degrading a polypeptide product comprising an enzyme composition of the invention.

Further provided is a method for producing the enzyme composition of the invention, said method comprising
(a) introducing into a host cell a nucleic acid encoding for
   i. a prolyl protease AfuS28 comprising SEQ ID NO: 1, a biologically active fragment thereof, a naturally occurring allelic variant thereof, or a sequence having at least 95% of identity, and
   ii. at least one tripeptidyl protease of the sedolisin family, said tripeptidyl protease selected from the group consisting in
      a) a sedolisin SedA comprising SEQ ID NO: 2, a biologically active fragment thereof, a naturally occurring allelic variant thereof, or a sequence having at least 95% of identity, or
      b) a sedolisin SedB comprising SEQ ID NO: 3, a biologically active fragment thereof, a naturally occurring allelic variant thereof, or a sequence having at least 95% of identity
      c) a sedolisin SedC comprising SEQ ID NO: 4, a biologically active fragment thereof, a naturally occurring allelic variant thereof, or a sequence having at least 95% of identity, or
      d) a sedolisin SedD comprising SEQ ID NO: 5, a biologically active fragment thereof, a naturally occurring allelic variant thereof, or a sequence having at least 95% of identity
(b) cultivating the cell of step (a) in a culture medium under conditions suitable for producing the enzyme composition; and
(c) recovering the enzyme composition.

### BRIEF DESCRIPTION OF FIGURES

FIG. 1: 10% SDS-PAGE stained with Coomassie blue of Aspergillus fumigatus secreted proteins at pH 3.5 and pH 7

FIG. 2: Distribution of proteases as a function of pH

FIG. 3: (a) 12% gel Coomassie Blue staining of recombinant AfuS28 Hist₆ Tag before and after deglycosylation.

(b) Western Blot of native and recombinant AfuS28 Hist₆ Tag deglycosilated

FIG. 4: Bradykinin degradation by AfuS28: the rectional medium contains 16 ml of Bradykinin, 0.02 nmol of AfuS28 Hist6 Tag and 0.05 mmol of Histidine on acidic buffer pH 4 (formic acid ˜0.0125%) and was incubated at 37° C. during 1 h. Reaction was stopped by adding 0.5% formic acid. All samples were diluted 10 times in H₂O:MeCN 50:50 (+0.1% formic acid) and infused in the LTQ-Orbitrap via the Nanomate.

FIG. 5: (a) Kinetics of 3-36 NPY degradation by AfuS28 during 15 min (1/2)

(b) Kinetics of 3-36 NPY degradation by AfuS28 during 15 min (2/2)

FIG. 6: NPY3-36 (a) and NPY1-36 (b) degradations by AfuS28 and SedB The rectional medium contains 4.8 nmol of NPY3-36 (a) or 1-36 (b), 0.02 nmol of AfuS28 Hist₆ Tag and/or 0.8 μg of SUB2 (or both of them) and 0.05 mmol of Histidine on acidic buffer pH 4 (Formic acid ˜0.0125%) and was incubated at 37° C. during 1 h. Reaction was stopped by adding 0.5% formic acid. All samples were diluted 10 times in H₂O:MeCN 50:50 (+0.1% formic acid) and infused in the LTQ-Orbitrap via the Nanomate. them) and 0.05 mmol of Histidine on acidic buffer pH 4 (Formic acid ˜0.0125%) and was incubated at 37° C. during 1 h. Reaction was stopped by adding 0.5% formic acid. All samples were diluted 10 times in H2O:MeCN 50:50 (+0.1% formic acid) and infused in the LTQ-Orbitrap via the Nanomate.

FIG. 7 shows degradation of gliadin by the enzyme composition AfuS28+SedB at pH 4.

FIG. 8 shows degradation of gliadin by the enzyme composition AfuS28+SedB at pH 8

Table 1: Primers for AfuS28 and AfuS28 antigen construct

Table 2: Proteases secreted massively by A. fumigatus on media containing collagen at pH 3.5 and 7 during 70-h growth under shaking at 30° C. Numbers of matched spectra give a semiquantitative measure of protein amounts.

Table 3: Comparison between secreted protein on pH 3.5 and 7 get by Shotgun proteomics analysis

Table 4: All theoretical and detected weight of peptides released after AfuS28 and SedB digestion of NPY1-36 and 3-36 by MS.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components.

As used in the specification and claims, the singular form “a”, “an” and “the” include plural references unless the context clearly dictates otherwise.

The term “endogenous” with reference to a polynucleotide or protein refers to a polynucleotide or protein that occurs naturally in the host cell.

The term “enzyme composition” is equivalent and interchangeable with the term “enzyme cocktail” or “enzyme combination” and refers to a mixture of more than one enzyme (protease in the context of the present invention) that digests for example proline rich peptides, proteins or polypeptides, such as gluten.

As used herein, the term “protease” is synonymous with peptidase, proteolytic enzyme and peptide hydrolase. The proteases include all enzymes that catalyse the cleavage of the peptide bonds (CO—NH) of proteins, digesting these proteins into peptides or free amino acids. Exopeptidases act near the ends of polypeptide chains at the amino (N) or carboxy (C) terminus. Those acting at a free N terminus liberate a single amino acid residue and are termed aminopeptidases.

Aspergillus fumigatus is an important opportunistic pathogen which is the main causative agent of invasive aspergillosis in neutropenic patients. Under natural conditions in composts, this fungus plays an important role in the decomposition of organic materials and in recycling environmental carbon and nitrogen. Like many other ascomycete fungi, A. fumigatus can grow in a medium containing protein as the sole nitrogen and carbon source. This ability to grow in a protein medium depends on the synergic action of secreted endo- and exoproteases since only amino acids and short peptides can be assimilated via membrane transporters. In contrast, large peptides cannot be used as nutrients. At neutral pH, A. fumigatus secrete two major endoproteases, an alkaline protease of the subtilisin family (Alp1) (Reichard et al., 1990; Monod et al. 1991) and a metalloprotease of the fungalysin family (Mep) (Monod et al., 1993a; 1993b; Jaton-Ogay et al., 1994), leucine aminopeptidases (Lap1 and Lap2) (Monod et al, 2005) and a X-prolyl peptidase (DppIV) (Beauvais et al., 1997). A similar battery of orthologue proteases was found to be secreted by Aspergillus oryzae (Doumas et al., 1998; 1999; Blinkowsky et al., 2000; Chien et al., 2002). With this set of enzymes, large peptides generated from proteins by endoproteolysis can be further digested into amino acids and X-pro dipeptides by the synergistic action of the leucine aminopeptidases and DppIV. Laps degrade peptides from their N-terminus till an X-Pro sequence which acts as a stop. However, in a complementary manner, X-Pro sequences can be removed by DppIV, which allows Laps an access to the following residues. Synergic action of A. oryzae Lap and DppIV at pH 7.5 was found to digest a peptide consisting of the sequence APGDRIYVHPF into amino acids, AP and HP di-peptides (Byun et al., 2001).

A. fumigatus also grows well in a protein medium at acidic pH like at neutral and basic pH. This is indicative that other enzymes are expressed at lower pH and are able to digest complex proteins in acidic conditions. The Applicants have shown that A. fumigatus secretes different sets of proteases at neutral and acidic pH, respectively. The Applicants have also described the different steps of protein digestion into assimilable amino acids and short peptides at acidic pH. In a protein medium at acidic pH, A. fumigatus was found to secrete a set of proteases which includes an aspartic protease of the pepsin family (Pep1) (as endoprotease), a glutamic protease (also as endoprotease), tripeptidyl-peptidases (Tpp) of the sedolisin family (SedB and SedD) (as exopeptidase), a prolyl-peptidase of the S28 family called AfuS28A (as exopeptidase) and carboxypeptidase of the S10 family (also as exopeptidase).

Proteomic investigation reveals that the fungus grows in a protein medium at neutral and acidic pH using two different set of secreted proteases. At neutral pH, the fungus secretes a set of neutral and alkaline proteases which includes Alp1, Mep1 as endoproteases and Laps, DppIV and AfuS28 as exoproteases. At acidic pH the fungus secretes another set of proteases which includes Pep and G1 as endoproteases and tripeptidyl-peptidases of the Sedolisin family and AfuS28 as exoproteases. During protein digestion the main function of endoproteases is to produce a large number of free ends on which exoproteases may act. The Applicants have shown that for example larges peptides such as NPY3-36 can be degraded from their N-terminus into amino acids, di- and tri-peptides by a synergic action of two peptidases, SedB and AfuS28.

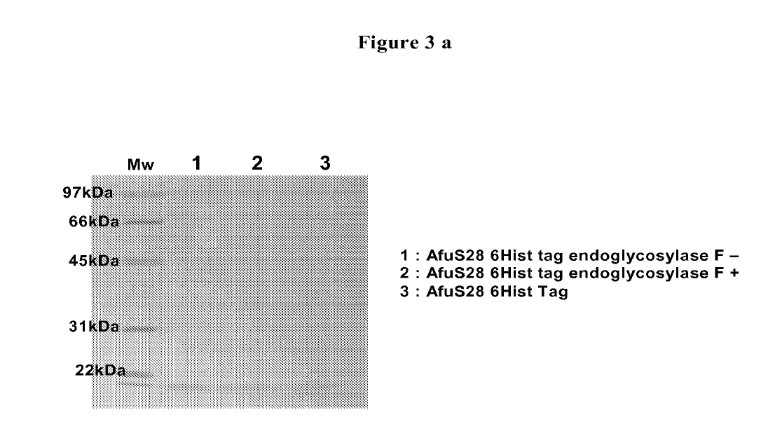

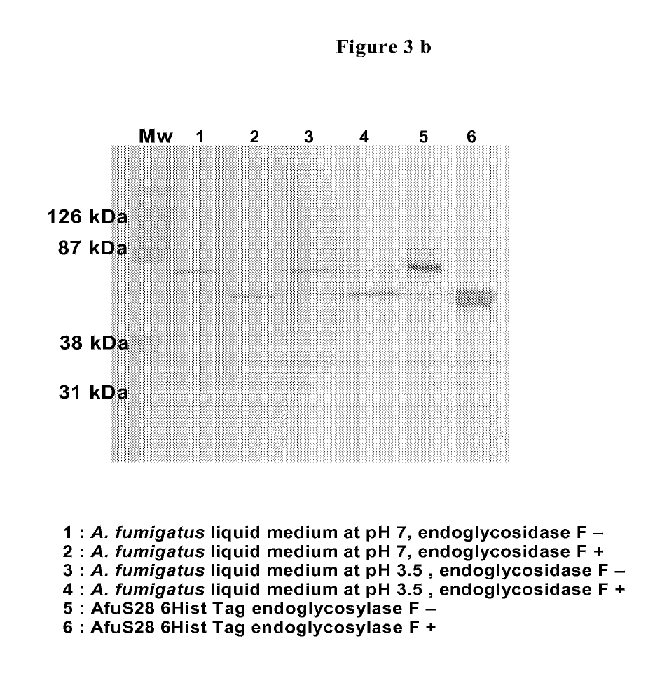

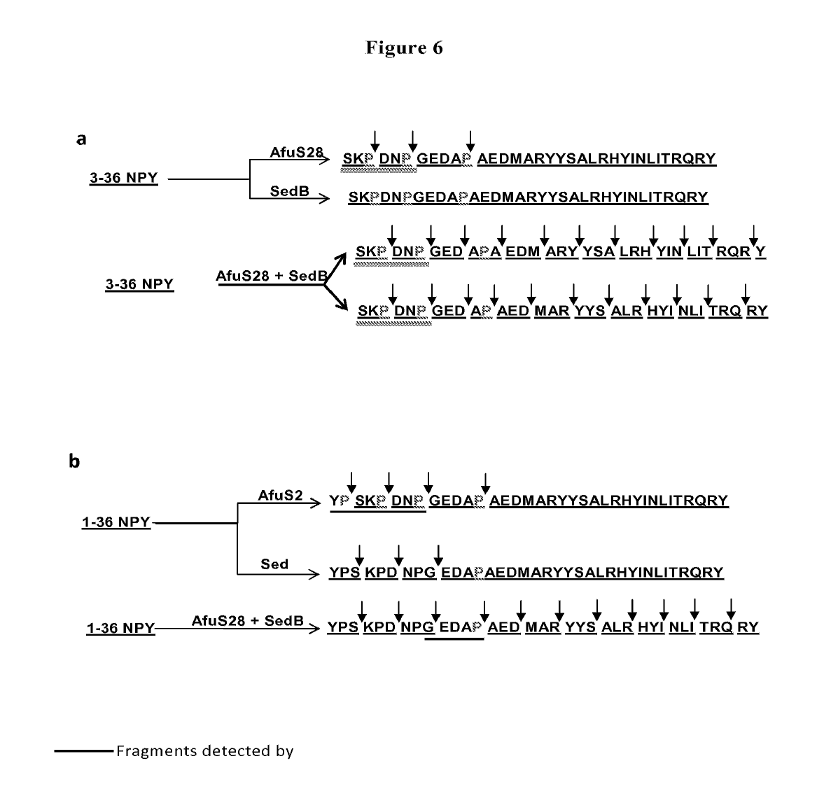

Among the 20 amino acids found in proteins, proline occupies a particular position because of its cyclic structure, and constitutes road blocks on the way of sequential protein hydrolysis by leucine aminopeptidases and tripepeptidyl-peptidases of the sedolisin family, at neutral and acidic pH, respectively (Byun et al., 2001; Monod et al., 2005; Reichard et al., 2006). However, both sets of proteases secreted by A. fumigatus contain exoproteases which allow the removing of proline residues in large peptide digestion. DppIV has the optimum active and is secreted at neutral pH, while still having a certain activity up to pH 4, whereas AfuS28 is active and secreted at neutral and acidic pH. Therefore, DppIV can be substituted by AfuS28 at neutral pH. In contrast, the latter peptidase may play a major function in peptide digestion from their N-terminus with tripeptidylpeptidases of the sedolisin family at acidic pH, since apparently A. fumigatus does not possesses other secreted prolyl exopeptidases (Monod et al., 2009). Only P residue in position P2 can be jumped by sedolisine enzymes which are active when amino acids in positions 3 and 4 from the N-terminus of the substrate peptide are not a proline (FIG. 6) (Reichard et al., 2006). Comparison between the A. fumigatus genome sequence and reverse transcriptase PCR products used to produce AfuS28 in P. pastoris showed that the AfuS28 gene consists of 10 exons. As a secreted protein, AfuS28 is synthesized as a preprotein precursor. The deduced amino acid sequence of the open reading frame encoded by the AfuS28 gene shows a 21-amino acid signal peptide with a hydrophobic core of 13 amino acid residues and a putative signal peptidase cleavage site Ala-Ser-Ala in accordance with the Von Heijne's rule (von Heijne 1986; Bentsen et al. 2004) The AfuS28 protein generated after signal peptidase cleavage is 504 amino acids long. The polypeptidic chain of the mature protein has a calculated molecular mass of 55 kDa, which is in accordance with that estimated for the deglycosylated protein by SDS-PAGE (FIG. 3a). The amino acid sequence of AfuS28 contains six potential N-linked glycosylation (Asn-X-Thr) sites, and the carbohydrate content of the secreted enzyme is about 20% (FIGS. 3a and 3b). AfuS28 contains a Gly-Gly-Ser-Tyr-Gly sequence (residue 173-177) in accordance with the consensus sequence Gly-X-Ser-X-Gly for the catalytic site of serine proteases. In addition to Ser 175, alignment of AfuS28 with afore cited S28 peptidases reveals Asp and H is residues of the catalytic triad in position 453 and 486, respectively. AfuS28 is closely related to A. niger prolylendopeptidase, which was described as a prolyl-endopeptidase, with around 75% identity.

The recombinant AfuS28 strictly hydrolyzed prolyl bonds but some bonds appear to be more resistant than others as evidenced by the accumulation of NPY 3-8 fragment (SKPDNP) during NPY3-36 digestion. In contrast to DppIV, AfuS28 is able to cleave peptides between and after two proline residues as revealed by products found from bradykinin digestion. A. niger prolylendopeptidase showed a specificity lower than that of AfuS28 being able to digest after amino acids other than proline (Kubota and al., 2005). Although AfuS28 cleaves substrates which are Z-blocked at the N-terminus, several facts support the conclusion that AfuS28 behaves rather as an Xn-prolyl exopeptidase. (i) AfuS28 does not attack full length protein substrates such as resorufin-labeled casein and BSA. (ii) NPY3-36 digestion was found to be sequentially performed from the N-terminus. AfuS28 and A. niger prolylendopeptidase are homologous to human lysosomal Pro-Xaa carboxypeptidase and DppII which have a substrate specificity similar to that of DppIV. While all proteases of the S28 family are specialized for hydrolyzing prolyl bonds, no crystal structure has yet been reported to understand the differences in substrate specificity in different members of the S28 family.

Gluten is a complex protein consisting of a mixture of numerous gliadin and glutenin polypeptides. Gluten proteins are rich in proline (15%) and glutamine (35%) residues, a feature that is especially notable among gluten epitopes that are recognized by disease-specific T cells. The principal toxic components of wheat gluten are a family of proline- and glutamine-rich proteins called gliadins, which are resistant to degradation in the gastrointestinal tract and contain several T-cell stimulatory epitopes (33 mer and 31-49 (p31-49) peptides). Proline rich nutriments such as glutens in cereals are highly resistant to proteolytic degradation in the gastrointestinal tract by pepsin, trypsin, chymotrypsin and the like.

Applicants have developed particular composition of proteases, which exhibits a proteolytic activity toward peptides, such as proline rich peptides, at acidic pH, which corresponds to the pH of the gastric fluid, and found that this enzyme composition is also able to degrade the 33 mer of the gliadin.

For example a combination of AfuS28 protease and at least one tripeptidyl protease of the sedolisin family sequentially digests a full length polypeptide chain and degrades a fragment of gliadin known to be resistant to protease action, thereby providing evidence that AfuS28 in combination with at least one tripeptidyl protease of the sedolisin family can be used for the treatment of celiac disease or any disease of the digestive tract such as malabsorption. The Applicants have shown that the co-incubation of gliadine with AfuS28 and SedB resulted in complete degradation of gliadin into short 2- to 5-mers.

AfuS28 in combination with at least one tripeptidyl protease of the sedolisin family and optionally with other proteases is also useful in the food industry, such as, but not limited to degrading substrates for bitterness, treatment of meat, soap industry, degrading prions, degrading viruses, and degrading toxic or contaminant proteins into short peptides and/or free amino acids.

Thus the present invention provides an enzyme composition, comprising
i. a prolyl protease AfuS28 comprising SEQ ID NO: 1, a biologically active fragment thereof, a naturally occurring allelic variant thereof, or a sequence having at least 95% of identity, and
ii. at least one tripeptidyl protease of the sedolisin family, said tripeptidyl protease is selected from the group consisting in
   a) a sedolisin SedA comprising SEQ ID NO: 2, a biologically active fragment thereof, a naturally occurring allelic variant thereof, or a sequence having at least 95% of identity, or
   b) a sedolisin SedB comprising SEQ ID NO: 3, a biologically active fragment thereof, a naturally occurring allelic variant thereof, or a sequence having at least 95% of identity, or
   c) a sedolisin SedC comprising SEQ ID NO: 4, a biologically active fragment thereof, a naturally occurring allelic variant thereof, or a sequence having at least 95% of identity, or
   d) a sedolisin SedD comprising SEQ ID NO: 5, a biologically active fragment thereof, a naturally occurring allelic variant thereof, or a sequence having at least 95% of identity

Preferably the enzyme composition of the invention comprises a prolyl protease AfuS28 comprising SEQ ID NO: 1, a biologically active fragment thereof, a naturally occurring allelic variant thereof, or a sequence having at least 95% of identity, and either a sedolisin SedB comprising SEQ ID NO: 3, a biologically active fragment thereof, a naturally occurring allelic variant thereof, or a sequence having at least 95% of identity, or a sedolisin SedD comprising SEQ ID NO: 5, a biologically active fragment thereof, a naturally occurring allelic variant thereof, or a sequence having at least 95% of identity, or a sedolisin SedC comprising SEQ ID NO: 4, a biologically active fragment thereof, a naturally occurring allelic variant thereof, or a sequence having at least 95% of identity.

The most preferably the enzyme composition of the invention comprises a prolyl protease AfuS28 comprising SEQ ID NO: 1, a biologically active fragment thereof, a naturally occurring allelic variant thereof, or a sequence having at least 95% of identity, and a sedolisin SedB comprising SEQ ID NO: 3, a biologically active fragment thereof, a naturally occurring allelic variant thereof, or a sequence having at least 95% of identity.

In a further embodiment, the enzyme composition of the invention comprises
i. a prolyl protease AfuS28 comprising SEQ ID NO: 1, a biologically active fragment thereof, a naturally occurring allelic variant thereof, or a sequence having at least 95% of identity,
ii. a sedolisin SedA comprising SEQ ID NO: 2, a biologically active fragment thereof, a naturally occurring allelic variant thereof, or a sequence having at least 95% of identity,
iii. a sedolisin SedB comprising SEQ ID NO: 3, a biologically active fragment thereof, a naturally occurring allelic variant thereof, or a sequence having at least 95% of identity
iv. a sedolisin SedC comprising SEQ ID NO: 4, a biologically active fragment thereof, a naturally occurring allelic variant thereof, or a sequence having at least 95% of identity, and
v. a sedolisin SedD comprising SEQ ID NO: 5, a biologically active fragment thereof, a naturally occurring allelic variant thereof, or a sequence having at least 95% of identity

The enzyme composition of the invention has an activity at pH values below 7 as well as slightly above 7 (pH 7 to 8). The optimum activity of the enzyme composition of the invention corresponds to the pH of the gastric fluid. Preferably the enzyme composition of the invention has an optimal activity at pH 2-4, and the most preferably at pH 2.5-3.5.

The term “acidic pH” or “low pH” corresponds to pH values below 7, which indicate an acid.

The enzyme composition of the invention further comprises optionally one or more proteases having activity at pH values below 7, said proteases being selected from the group comprising:
an aspartic protease of the pepsin family (Pep1) comprising SEQ ID NO: 6, a biologically active fragment thereof, a naturally occurring allelic variant thereof, or a sequence having at least 95% of identity.
a glutamic protease serine comprising SEQ ID NO: 7, a biologically active fragment thereof, a naturally occurring allelic variant thereof, or a sequence having at least 95% of identity.
carboxypeptidase Scp1 comprising SEQ ID NO:8, a biologically active fragment thereof, a naturally occurring allelic variant thereof, or a sequence having at least 95% of identity, and
X-prolyl peptidase (DppIV) comprising SEQ ID NO:9, a biologically active fragment thereof, a naturally occurring allelic variant thereof, or a sequence having at least 95% of identity.

Preferably, the enzyme composition of the invention comprises additionally X-prolyl peptidase (DppIV) comprising SEQ ID NO:9, a biologically active fragment thereof, a naturally occurring allelic variant thereof, or a sequence having at least 95% of identity.

As herein used the term “protease of the invention” or “proteases of the invention” is a protease or proteases of the enzyme composition of the present invention.

The following sequences are considered in the present invention:

SEQ ID NO: 1
MRTAAASLTLAATCLFELASALMPRAPLIPAMKAKVALPSGNATFEQYIDHNNPGLG
TFPQRYWYNPEFWAGPGSPVLLFTPGESDAADYDGFLTNKTIVGRFAEEIGGAVILLE
HRYWGASSPYPELTTETLQYLTLEQSIADLVHFAKTVNLPFDEIHSSNADNAPWVMT
GGSYSGALAAWTASIAPGTFWAYHASSAPVQAIYDFWQYFVPVVEGMPKNCSKDL
NRVVEYIDHVYESGDIERQQEIKEMFGLGALKHFDDFAAAITNGPWLWQDMNFVSG
YSRFYKFCDAVENVTPGAKSVPGPEGVGLEKALQGYASWFNSTYLPGSCAEYKYW
TDKDAVDCYDSYETNSPIYTDKAVNNTSNKQWTWFLCNEPLFYWQDGAPKDEST
IVSRIVSAEYWQRQCHAYFPEVNGYTFGSANGKTAEDVNKWTKGWDLTNTTRLIW
ANGQFDPWRDASVSSKTRPGGPLQSTEQAPVHVIPGGFHCSDQWLVYGEANAGVQ
KVIDEEVAQIKAWVAEYPKYRKP
SEQ ID NO: 2
MRLSHVLLGTAAAAGVLASPTPNDYVVHERRAVLPRSWTEEKRLDKASILPMRIGLTQS
NLDRGHDLLMEISDPRSSRYGQHLSVEEVHSLFAPSQETVDRVRAWLESEGIAGDRISQS
SNEQFLQFDASAAEVERLLGTEYYLYTHQGSGKSHIACREYHVPHSLQRHIDYITPGIKL
LEVEGVKKARSIEKRSFRSPLPPILERLTLPLSELLGNTLLCDVAITPLCISALYNITRGSKA
TKGNELGIFEDLGDVYSQEDLNLFFSTFAQQIPQGTHPILKAVDGAQAPTSVTNAGPESD
LDFQISYPIIWPQNSILFQTDDPNYTANYNFSGFLNTFLDAIDGSYCSEISPLDPPYPNPAD
GGYKGQLQCGVYQPPKVLSISYGGAEADLPIAYQRRQCAEWMKLGLQGVSVVVASGD
SGVEGRNGDPTPTECLGTEGKVFAPDFPATCPYLTTVGGTYLPLGADPRKDEEVAVTSF
PSGGGFSNIYERADYQQQAVEDYFSRADPGYPFYESVDNSSFAENGGIYNRIGRAYPDV
AAIADNVVIFNKGMPTLIGGTSAAAPVFAAILTRINEERLAVGKSTVGFVNPVLYAHPEV
FNDITQGSNPGCGMQGFSAATGWDPVTGLGTPNYPALLDLFMSLP
SEQ ID NO: 3
MFSSLLNRGALLAVVSLLSSSVAAEVFEKLSAVPQGWKYSHTPSDRDPIRLQIALKQ
HDVEGFETALLEMSDPYHPNYGKHFQTHEEMKRMLLPTQEAVESVRGWLESAGISD
IEEDADWIKFRTTVGVANDLLDADFKWYVNEVGHVERLRTLAYSLPQSVASHVNM
VQPTTRFGQIKPNRATMRGRPVQVDADILSAAVQAGDTSTCDQVITPQCLKDLYNIG
DYKADPNGGSKVAFASFLEEYARYDDLAKFEEKLAPYAIGQNFSVIQYNGGLNDQN
SASDSGEANLDLQYIVGVSSPIPVTEFSTGGRGLLIPDLSQPDPNDNSNEPYLEFLQNV
LKMDQDKLPQVISTSYGEDEQTIPEKYARSVCNLYAQLGSRGVSVIFSSGDSGVGAA
CLTNDGTNRTHFPPQFPAACPWVTSVGGTTKTQPEEAVYFSSGGFSDLWERPSWQD
SAVKRYLKKLGPRYKGLYNPKGRAFPDVAAQAENYAVFDKGVLHQFDGTSCSAPA
FSAIVALLNDARLRAHKPVMGFLNPWLYSKASKGFNDIVKGGSKGCDGRNRFGGTP
NGSPVVPYASWNATDGWDPATGLGTPDFGKLLSLAMRR
SEQ ID NO: 4
MAPFTFLVGILSLCICCIVLGAAAEPSYAVVEQLRNVPDGWIKHDAAPASELIRFRLA
MNQERAAEFERRVIDMSTPGHSSYGQHMKRDDVREFLRPPEEVSDKVLSWLRSENV
PAGSIESHGNWVTFTVPVSQAERMLRTRFYAFQHVETSTTQVRTLAYSVPHDVHRYI
QMIQPTTRFGQPARHERQPLFHGTVATKEELAANCSTTITPNCLRELYGIYDTRAEPD
PRNRLGVSGFLDQYARYDDFENFMRLYATSRTDVNFTVVSINDGLNLQDSSLSSTEA
SLDVQYAYSLAYKALGTYYTTGGRGPVVPEEGQDTNVSTNEPYLDQLHYLLDLPDE
ELPAVLSTSYGEDEQSVPESYSNATCNLFAQLGARGVSIIFSSGDSGVGSTCITNDGTK
TTRFLPVFPASCPFVTAVGGTHDIQPEKAISFSSGGFSDHFPRPSYQDSSVQGYLEQLG
SRWNGLYNPSGRGFPDVAAQATNFVVIDHGQTLRVGGTSASAPVFAAIVSRLNAAR
LEDGLLKLGFLNPWLYSLNQTGFTDIIDGGSSGCYVGTSNEQLVPNASWNATPGWD
PVTGLGTPIYNTLVKLATSVSSTP
SEQ ID NO: 5
MLSSTLYAGWLLSLAAPALCVVQEKLSAVPSGWTLIEDASESDTITLSIALARQNLD
QLESKLTTLATPGNPEYGKWLDQSDIESLFPTASDDAVLQWLKAAGITQVSRQGSLV
NFATTVGTANKLFDTKFSYYRNGASQKLRTTQYSIPDHLTESIDLIAPTVFFGKEQNS
ALSSHAVKLPALPRRAATNSSCANLITPDCLVEMYNLGDYKPDASSGSRVGFGSFLN
ESANYADLAAYEQLFNIPPQNFSVELINRGVNDQNWATASLGEANLDVELIVAVSHP
LPVVEFITGGSPPFVPNADEPTAADNQNEPYLQYYEYLLSKPNSHLPQVISNSYGDDE
QTVPEYYARRVCNLIGLMGLRGITVLESSGDTGIGSACMSNDGTNKPQFTPTFPGTCP
FITAVGGTQSYAPEVAWDGSSGGFSNYFSRPWYQSFAVDNYLNNHITKDTKKYYSQ
YTNFKGRGFPDVSAHSLTPYYEVVLTGKHYKSGGTSAASPVFAGIVGLLNDARLRA
GKSTLGFLNPLLYSILAEGFTDITAGSSIGCNGINPQTGKPVPGGGIIPYAHWNATAG
WDPVTGLGVPDFMKLKELVLSL
SEQ ID NO: 6
MVVFSKVTAVVVGLSTIVSAVPVVQPRKGFTINQVARPVTNKKTVNLPAVYANALTKY
GGTVPDSVKAAASSGSAVTTPEQYDSEYLTPVKVGGTTLNLDFDTGSADLWVFSSELSA
SQSSGHAIYKPSANAQKLNGYTWKIQYGDGSSASGDVYKDTVTVGGVTAQSQAVEAA
SHISSQFVQDKDNDGLLGLAFSSINTVSPRPQTTFFDTVKSQLDSPLFAVTLKYHAPGTY
DFGYIDNSKFQGELTYTDVDSSQGFWMFTADGYGVGNGAPNSNSISGIADTGTTLLLLD
DSVVADYYRQVSGAKNSNQYGGYVFPCSTKLPSFTTVIGGYNAVVPGEYINYAPVTDG
SSTCYGGIQSNSGLGFSIFGDIFLKSQYVVFDSQGPRLGFAPQA
SEQ ID NO: 7
MKFTSVLASGLLATAAIAAPLTEQRQARHARRLARTANRSSHPPYKPGTSEVIKLSN
TTQVEYSSNWAGAVLIGTGYTAVTGEFVVPTPSVPSGGSSSKQYCASAWVGIDGDT
CSSAILQTGVDFCIQGSSVSFDAWYEWYPDYAYDFSGISISAGDTIRVTVDATSKTAG
TATVENVTKGKTVTHTFTGGVDGNLCEYNAEWIVEDFESNGSLVPFANFGTVTFTG
AQATDGGSTVGPSGATLIDIQQSGKVLTSVSTSSSSVTVKYV
SEQ ID NO: 8
MLSLVTLLSGTAGLALTASAQYFPPTPEGLKVVHSKHQEGVKISYKEPGICETTPGVK
SYSGYVHLPPGTLNDVDVDQQYPINTFFCFFESRNDPIHAPLAIWMNGGPGSSSMIGL
LQENGPCLVNADSNSTEINPWSWNNYVNMLYIDQPNQVGFSYDVPTNGTYNQLTTA
WNVSAFPDGKVPEQNNTFYVGTFPSMNRTATANTTQNAARSLWHFAQTWFSEFPE
YKPHDDRVSIWTESYGGRYGPSFAAFFQEQNEKIEEGALPDEYHYIHLDTLGIINGCV
DLLTQAPFYPDMAYNNTYGIEAINKTVYERAMNAWSKPGGCKDLIVKCRELAAEGD
PTMSGHNETVNEACRRANDYCSNQVEGPYILFSKRGYYDIAHFDPDPFPPPYFQGFL
NQNWVQAALGVPVNFSISVDSTYSAFASTGDYPRADVHGYLEDLAYVLDSGIKVAL
VYGDRDYACPWNGGEEVSLRVNYSDSQSFQKAGYAPVQTNSSYIGGRVRQYGNFSF
TRVFEAGHEVPAYQPQTAYEIFHRALFNRDIATGKMSLLKNATYASEGPSSTWEFKN
EVPESPEPTCYIQSLQSSCTEEQIQSVVNGTALIKDWIVVEKVDIY
SEQ ID NO: 9
MKWSILLLVGCAAAIDVPRQPYAPTGSGKKRLTFNETVVKRAISPSAISVEWISTSED
GDYVYQDQDGSLKIQSIVTNHTQTLVPADKVPEDAYSYWIHPNLSSVLWATNYTKQ
YRHSYFADYFIQDVQSMKLRPLAPDQSGDIQYAQWTPTGDAIAFVRDNNVFVWTNA
STSQITNDGGPDLFNGVPDWIYEEEILGDRFALWFSPDGAYLAFLRFNETGVPTFTVP
YYMDNEEIAPPYPRELELRYPKVSQTNPTVELNLLELRTGERTPVPIDAFDAKELIIGE
VAWLTGKHDVVAVKAFNRVQDRQKVVAVDVASLRSKTISERDGTDGWLDNLLSM
AYIGPIGESKEEYYIDISDQSGWAHLWLFPVAGGEPIALTKGEWEVTNILSIDKPRQL
VYFLSTKHHSTERHLYSVSWKTKEITPLVDDTVPAVWSASFSSQGGYYILSYRGPDV
PYQDLYAINSTAPLRTITSNAAVLNALKEYTLPNITYFELALPSGETLNVMQRLPVKF
SPKKKYPVLFTPYGGPGAQEVSKAWQALDFKAYIASDPELEYITWTVDNRGTGYKG
RAFRCQVASRLGELEAADQVFAAQQAAKLPYVDAQHIAIWGWSYGGYLTGKVIET
DSGAFSLGVQTAPVSDWRFYDSMYTERYMKTLESNAAGYNASAIRKVAGYKNVRG
GVLIQHGTGDDNVHFQNAAALVDTLVGAGVTPEKLQVQWFTDSDHGIRYHGGNVF
LYRQLSKRLYEEKKRKEKGEAHQWSKKSVL

A protease of the invention includes a protease comprising the amino acid sequence comprising SEQ ID NOs: 1, 2, 3, 4, 5, 6, 7, 8, and 9. The invention also includes a mutant or variant protease any of whose residues may be changed from the corresponding residues shown in SEQ ID NOs: 1, 2, 3, 4, 5, 6, 7, 8 or 9 while still maintaining its activity and physiological functions, or a biologically active fragment thereof.

The present invention is also directed to variants of proteases of the invention. The term “variant” refers to a polypeptide or protein having an amino acid sequence that differs to some extent from a native SEQ ID NOs: 1, 2, 3, 4, 5, 6, 7, 8 or 9, and which is an amino acid sequence that vary from the native sequence by conservative amino acid substitutions, whereby one or more amino acids are substituted by another with same characteristics and conformational roles. The amino acid sequence variants possess substitutions, deletions, side-chain modifications and/or insertions at certain positions within the amino acid sequence of the native amino acid sequence. Conservative amino acid substitutions are herein defined as exchanges within one of the following five groups:

I. Small aliphatic, nonpolar or slightly polar residues: Ala, Ser, Thr, Pro, Gly
II. Polar, positively charged residues: H is, Arg, Lys
III. Polar, negatively charged residues: and their amides: Asp, Asn, Glu, Gln
IV. Large, aromatic residues: Phe, Tyr, Trp
V. Large, aliphatic, nonpolar residues: Met, Leu, Ile, Val, Cys.

In another aspect, the present invention is directed to isolated proteases of the invention, and biologically active fragments thereof (or derivatives, portions, analogs or homologs thereof). Biologically active fragment refers to regions of the proteases of the invention, which are necessary for normal function, for example, prolyl, sedolisin, pepsin, glutamic or carboxypeptidase like protease activities. Biologically active fragments include peptides comprising amino acid sequences sufficiently homologous to or derived from the amino acid sequences of SEQ ID NOs: 1, 2, 3, 4, 5, 6, 7, 8 or 9, that include fewer amino acids than the full-length protease, and exhibit at least one activity of a protease of the invention. Typically, biologically active fragments comprise a domain or motif with at least one activity of the protease of the invention. A biologically active fragment of a protease of the invention can be a polypeptide that is, for example, 10, 25, 50, 100 or more amino acid residues in length. Moreover, other biologically active fragments, in which other regions of the protease are deleted, can be prepared by recombinant techniques and evaluated for one or more of the functional activities of a native protease of the invention.

In a further embodiment, the protease of the invention is a protease that comprises an amino acid sequence having at least 70%, 80%, 90%, 95% or 99%, preferably 95%, identity to the amino acid sequence comprising SEQ ID NOs: 1, 2, 3, 4, 5, 6, 7, 8 or 9 and retains the activity of the proteases comprising SEQ ID NOs: 1, 2, 3, 4, 5, 6, 7, 8 or 9.

To determine the percent of identity or homology of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “identity” is equivalent to amino acid or nucleic acid “homology”). The alignment and the percent homology or identity can be determined using any suitable software program known in the art, for example those described in CURRENT PROTOCOLS IN MOLECULAR BIOLOGY (F. M. Ausubel et al. (eds) 1987, Supplement 30, section 7.7.18). Preferred programs include the GCG Pileup program, FASTA (Pearson et al. (1988) Proc. Natl, Acad. Sci. USA 85:2444-2448), and BLAST (BLAST Manual, Altschul et al., Natl. Cent. Biotechnol. Inf., Natl Lib. Med. (NCIB NLM NIH), Bethesda, Md., and Altschul et al., (1997) NAR 25:3389-3402). Another preferred alignment program is ALIGN Plus (Scientific and Educational Software, PA), preferably using default parameters. Another sequence software program that finds use is the TFASTA Data Searching Program available in the Sequence Software Package Version 6.0 (Genetics Computer Group, University of Wisconsin, Madison, Wis.).

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (e.g., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

The invention also provides proteases of the invention as chimeric or fusion proteins. As used herein, a “chimeric protein” or “fusion protein” of proteases of the invention comprises a protease of the invention operatively-linked to another polypeptide. A protease of the invention refers to a polypeptide having an amino acid sequence corresponding to a SEQ ID NOs: 1, 2, 3, 4, 5, 6, 7, 8 or 9, whereas “another polypeptide” refers to a polypeptide having an amino acid sequence corresponding to a protein that is not substantially homologous to the protease of the invention, e.g., a protein that is different from the protease of the invention and that is derived from the same or a different organism. Within a fusion protein, the polypeptide can correspond to all or a portion of a protease of the invention. In one embodiment, a fusion protein comprises at least one biologically active fragment of a protease of the invention. In another embodiment, a fusion protein comprises at least two biologically active fragments of a protease of the invention. In yet another embodiment, a fusion protein comprises at least three biologically active fragments of a protease of the invention. Within the fusion protein, the term “operatively-linked” is intended to indicate that the polypeptide of a protease of the invention and another polypeptide are fused in-frame with one another. Another polypeptide can be fused to the N-terminus and/or C-terminus of the polypeptide of protease of the invention. In one embodiment, the fusion protein is a GST fusion protein in which the sequences of the protease of the invention are fused to the C-terminus of the GST (glutathione S-transferase) sequences. Such fusion proteins can facilitate the purification of recombinant protease of the invention. In another embodiment, the fusion protein is a protease of the invention containing a heterologous signal sequence at its N-terminus. In certain host cells (e.g., mammalian host cells), expression and/or secretion of proteases of the invention can be increased through use of a heterologous signal sequence. A chimeric or fusion protein of the invention can be produced by standard recombinant DNA techniques or conventional techniques including automated DNA synthesizers. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation.

The proteases of the enzyme composition of the invention operate with a synergic action. The Applicants have shown for example that larges peptides such as NPY3-36 can be degraded at acidic pH from their N-terminus into amino acids, di- and tri-peptides by a synergic action of two proteases, AfuS28 and SedB. AfuS28 protease plays a major function in peptide digestion from their N-terminus with tripeptidylpeptidases of the sedolisin family at acidic pH. Only P residue in position P2 can be jumped by Sedolisines which are active when amino acids in positions 3 and 4 from the N-terminus of the substrate peptide are not a proline (FIG. 6) (Reichard et al., 2006).

Large peptide NPY1-36 was not digested by only SedB at acidic pH, but this enzyme removed tripeptides NPY1-3, NPY4-6 and NPY7-9 (YPS, KPD and NPG) from the N-terminus of NPY1-36 until position 10 (FIG. 6). SedB appeared to be active only when the amino acid in P1 or P′ l position (amino acids in positions 3 and 4 from the N-terminus of any substrate peptide) was not a proline. AfuS28 and SedB added together degraded NPY3-36 in Y, di- and tri-peptides (FIG. 6, Table 4). Two different ways of degradation could be reconstituted. In the first way, SedB cleaves NPY9-36 (NPY9XXX-P-(X)23 (generated by AfuS28) in tri-peptides (and jumped P13). In the second way, AfuS28 first acts on P13 before further SedB digestion. Other tripeptides such as NPY28-30, NPY31-33 and NPY34-36 INL, ITR or QRY which would result from other ways of degradation were not detected.

The present invention further relates to a pharmaceutical composition comprising the enzyme composition of the invention and at least one pharmaceutically acceptable excipient, carrier and/or diluent. A pharmaceutical composition of the invention is formulated to be compatible with its intended route of administration, which is preferably the oral administration. For example, a crude preparation of cell culture medium from Aspergillus fumigatus or transgenic fungi producing the enzyme composition of the invention, or the enzyme composition purified from Aspergillus fumigatus can be administered orally since the proteases of the invention are secreted.

For oral administration, the enzyme composition of the invention may be formulated for example in the form of capsules (coated or non-coated) containing powder, coated or non-coated pellets, granules or micro-/mini-tablets or in the form of tablets (coated or non-coated) pressed from powder, coated or non-coated pellets, dragees or micro-/mini-tablets, hydrogels, liposomes, nanosomes, encapsulation, PEGylation. The enzyme composition of the invention may also be formulated for example in the form of gel caps or in liquid form as solution, drops, suspension or gel also be formulated e.g. as dried or moist oral supplement. The formulation of the enzyme composition according to the present invention as powder is particularly suitable for admixing with foodstuff. The powder may be sprinkled onto a meal or mixed into a pulp or beverage. It is particularly beneficial, if the enzyme composition offered as bulk powder is packaged in single dosage amounts, such as in single bags or capsules, or if it is provided in a dosing dispenser.

Suitable excipients, carriers and/or diluents include maltodextrin, cyclodextrines, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellu-lose, povidone, carboxymethyl cellulose, corn starch, modified starch, fibersol, gelatine, hy-droxypropylmethyl cellulose and the like (including mixtures thereof). Preferable carriers include calcium carbonate, magnesium stearate, maltodex-trin, dicalcium phosphate, modified starch, microcrystalline cellulose, fibersol, gelatine, hydroxypropylmethyl cellulose and mixtures thereof.

The various ingredients and the excipient, carrier and/or diluent may be mixed and formed into the desired form using common methods well known to the skilled person. The administration form according to the present invention which is suited for the oral route, such as e.g. tablet or capsule, may be coated with a coating which is resistant against low pH values (approximately pH 1 to 2.5) and which dissolves at a pH value of approximately 3.0 to 8.0, preferably at a pH value of 3.0 to 6.5 and particularly preferable at a pH value of 4.0 to 6.0. An optionally used coating should be in accordance with the pH optimum of the enzyme composition used and its stability at pH values to which the formulation will be exposed. Also a coating may be used which is not resistant to low pH values but which delays the release of the enzyme composition at low pH values. It is also possible to prepare the enzyme composition according to the present invention as coated (see above) pellets, granules or micro-/mini-tablets which can be filled into coated or non-coated capsules or which can be pressed into coated or non-coated tablets. Suitable coatings are, for example, cellulose acetate phthalate, cellulose deri-vates, shellac, polyvinylpyrrolidone derivates, acrylic acid, poly-acrylic acid derivates and polymethyl methacrylate (PMMA), such as e.g. Eudragit® (from Rohm GmbH, Darmstadt, Germany), in particular Eudragit® L30D-55. The coating Eudragit® L30D-55 is dissolved, for example, at a pH value of 5.5 and higher. If it is desired to release the enzyme composition already at a lower pH value, this may be achieved e.g. by the addition of sodium hydroxide solution to the coating agent Eudragit® L30D-55, because in this case carboxyl groups of the methacrylate would be neutralised. Therefore, this coating will be dissolved, for example, already at a pH value of 4.0 provided that 5% of the carboxyl groups are neutralised. The addition of about 100 g of 4% sodium hydroxide solution to 1 kg of Eudragit® L30D-55 would result in a neutralisation of about 6% of the carboxyl groups. Further details about formulation methods and administration methods can be found in the 21^{st} edition of “Remington: The Science & Practice of Pharmacy”, published 2005 by Lippincott, Williams & Wilkins, Baltimore, USA, in the Encyclopedia of Pharmaceutical Technology (Editor James Swarbrick) and in Prof. Bauer “Lehrbuch der Pharmazeutischen Technologie”, 18th edition, published 2006 by Wissenschaftliche Verlagsgesellschaft (ISBN 3804-72222-9). The contents of these documents are incorporated herein by reference.

Other suitable acceptable excipients, carriers and/or diluents for use in the present invention include, but are not limited to water, mineral oil, ethylene glycol, propylene glycol, lanolin, glyceryl stearate, sorbitan stearate, isopropyl myristate, isopropyl palmitate, acetone, glycerine, phosphatidylcholine, sodium cholate or ethanol.

The pharmaceutical compositions for use in the present invention may also comprise at least one co-emulsifying agent which includes but is not limited to oxyethylenated sorbitan monostearate, fatty alcohols, such as stearyl alcohol or cetyl alcohol, or esters of fatty acids and polyols, such as glyceryl stearate.

The enzyme composition according to the present invention may be provided in a stabilized form. Generally, stabilization methods and procedures which may be used according to the present invention include any and all methods for the stabilization of chemical or biological material which are known in the art, comprising e.g. the addition of chemical agents, methods which are based on temperature modulation, methods which are based on irradiation or combinations thereof. Chemical agents that may be used according to the present invention include, among others, preservatives, acids, bases, salts, antioxidants, viscosity enhancers, emulsifying agents, gelatinizers, and mixtures thereof.

In cases of treating the celiac disease, the pharmaceutical compositions employed are preferably formulated so as to release their activity in gastric fluid. This type of formulations will provide optimum activity in the right place, i.e. the release of the proteases of the invention in stomach

The dosage unit form of the pharmaceutical composition may be chosen from among a variety of such forms. In the case of tablets, capsules etc. the weight of each dosage unit is usually less than 0.5 g, these dosage units being intended for administration in an amount of say 1 to 2 tablets (to be ingested before, during or after meals) e.g. 2 to 3 times per day.

The pharmaceutical composition according to the present invention will normally contain the enzyme composition of the invention in an amount of from 0.0001 to 100% (w/w), e.g. from 0.001 to 90% (w/w). The exact amount will depend on the particular type of composition employed and on the specific protease activity per mg of protein.

As regards the protease activity in the pharmaceutical composition, this will often be within a range of from 0.1 to 0.0001 enzyme units per mg; but in some cases other activity per mg ranges may be obtained, depending on the purity of the enzyme preparation.

The pharmaceutical compositions can be included in a container, pack, or dispenser together with instructions for administration.

The present invention further provides a food supplement comprising the enzyme composition of the present invention. The term “food supplement” in the context of the present invention is equivalent and interchangeable with the terms food additive, a dietary supplement, alicament, and nutritional supplement.

In the food supplement of the invention, a carrier material is commonly added, although not essential, to the enzyme composition. Suitable carrier materials include maltodextrins, modified starches, direct compression tablet excipients such as dicalcium phosphate, calcium sulfate and sucrose. A particularly preferred carrier ingredient is the 10 DE Maltrin M100 maltodextrin from Grain Processing Corporation. Carriers can be added in concentrations ranging from 50 to 95 weight percent of the total composition.

The enzyme composition according to the present invention may contain the enzymes without further additives. However, it is preferable that the enzyme composition according to the present invention further contains additives that are pharmaceutically acceptable and/or acceptable for food supplements, such as for example extenders, binders, stabilizers, preservatives, flavourings, etc. Such additives are commonly used and well known for the production of pharmaceutical compositions, medical devices, food supplements, and special food supplements and the person skilled in the art knows which additives in which amounts are suitable for certain presentation forms. The enzyme composition according to the present invention may for example contain as additives dicalcium phosphate, lactose, modified starch, microcrystal-line cellulose, maltodextrin and/or fibersol.

The food supplement of the invention may be a granulated enzyme product which may readily be mixed with food components. Alternatively, food supplements of the invention can form a component of a pre-mix. The granulated enzyme composition product of the invention may be coated or uncoated. The particle size of the enzyme granulates can be compatible with that of food and pre-mix components. This provides a safe and convenient mean of incorporating enzymes into food supplements. Alternatively, the food supplements of the invention may be a stabilized liquid composition. This may be an aqueous or oil-based slurry.

In another aspect, enzyme composition of the invention can be supplied by expressing the enzymes directly in transgenic food crops (as, e.g., transgenic plants, seeds and the like), such as grains, cereals, corn, soy bean, rape seed, lupin and the like. For example transgenic plants, plant parts and plant cells can comprise nucleic acids encoding the proteases of the invention. In one aspect, the nucleic acid is expressed such that the enzyme (e.g., AfuS28) of the invention is produced in recoverable quantities. The enzyme composition of the invention can be recovered from any plant or plant part. Alternatively, the plant or plant part containing the recombinant polypeptide can be used as such for improving the quality of a food, e.g., improving nutritional value, palatability, and rheological properties, or to destroy an antinutritive factor.

The pharmaceutical composition or the food supplement of the invention can be provided at a time of a meal so that the proteases of the enzyme composition are released or activated in the upper gastrointestinal lumen where the proteases can complement gastric and pancreatic enzymes to detoxify ingested gluten and prevent harmful peptides to reach the mucosal surface. The enzyme composition according to the present invention can be taken orally prior to meals, immediately before meals, with meals or immediately after meals, so that it can exert its proteolytic effect on proline-rich nutriments in the food pulp. For example the extract from a wild type Aspergillus strain or from an engineered strain of Aspergillus to produce the enzyme composition of the invention could be used as a food supplement before a gluten rich meal in celiac disease.

Celiac disease (CD) is a digestive genetically determined disorder that damages the small intestine and interferes with absorption of nutrients from food. People who have CD cannot tolerate a protein called gluten, which is found in wheat, rye and barley. The disease has a prevalence of about 1:200 in most of the world's population groups and the only treatment for CD is to maintain a life-long, strictly gluten-free diet. For most people, following this diet will stop symptoms, heal existing intestinal lesions, and prevent further damage. The disease is more frequent in the paediatric population. Patients are suspected of having CD when they are presenting gastrointestinal or malabsorption symptoms. The principal toxic components of wheat gluten are a family of proline- and glutamine-rich proteins called gliadins, which are resistant to degradation in the gastrointestinal tract and contain several T-cell stimulatory epitopes (33 mer and 31-49 (p31-49) peptides). The 33-mer peptide is an excellent substrate for the enzyme transglutaminase 2 (TG2) that deamidates the immunogenic gliadin peptides, increasing their affinity to human leucocyte antigen (HLA) DQ2 or DQ8 molecules and thus activating the T cell-mediated mucosal immune response leading to clinical symptoms. The toxicity of these fragments may be due to an overexpression of transferrin receptor in CD allowing intestinal transport of intact peptide across the enterocyte. Thus the peptides can escape degradation by the acidic endosome-lysosomal pathway only in patients with active CD and can reach the serosal border unchanged.

Since in patients with celiac disease the gastrointestinal tract does not possess the enzymatic equipment to efficiently cleave the gluten-derived proline-rich peptides, driving the abnormal immune intestinal response, another therapeutic approach relies on the use of orally active proteases to degrade toxic gliadin peptides before they reach the mucosa. Oral therapy by exogenous prolyl-endopeptidases able to digest ingested gluten is therefore propounded as an alternative treatment to the diet.

Thus the enzyme composition of the invention is provided for use in a method for treating and/or preventing a syndrome associated with a human disease, said disease being selected from the group comprising celiac disease, digestive tract bad absorption, an allergic reaction, an enzyme deficiency, a fungal infection, Crohn disease, mycoses and sprue. The allergic reaction is a reaction to gluten or fragments thereof. Preferably a fragment of gluten is gliadine.

The present invention also relates to a method for treating and/or preventing a syndrome associated with a human disease in a subject suffering therefrom comprising administering a therapeutically effective amount of the enzyme composition of the present invention or the pharmaceutical composition of the present invention, said disease being selected from the group comprising celiac disease, digestive tract bad absorption, an allergic reaction, an enzyme deficiency, a fungal infection, Crohn disease, mycoses and sprue.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder, such as celiac disease, digestive tract bad absorption, an allergic reaction, an enzyme deficiency, a fungal infection, Crohn disease, mycoses and sprue. The term does not denote a particular age or sex. Thus, adult and newborn subjects, whether male or female, are intended to be covered.

The present invention further relates to a method of detoxifying gliadin comprising contacting gliadin containing food product with an effective dose of the enzyme composition of the invention. The term “food product”, “foodstuff” or “food” encompasses also any proline rich nutriment, such as gluten.

In one aspect, treating food products using the enzyme composition of the invention can help in the availability of nutrients, e.g., starch, protein, and the like, in the food product. By breaking down difficult to digest proteins, such as gluten, or indirectly or directly unmasking starch (or other nutrients), the enzyme composition of the invention makes nutrients more accessible to other endogenous or exogenous enzymes. The enzyme composition of the invention can also simply cause the release of readily digestible and easily absorbed nutrients and sugars. When added to food products, the enzyme composition of the invention improve the in vivo break-down of plant cell wall material partly due to a reduction of the intestinal viscosity (see, e.g., Bedford et al., Proceedings of the 1st Symposium on Enzymes in Animal Nutrition, 1993, pp. 73-77), whereby a better utilization of the plant nutrients by the mammal is achieved.

The present invention further provides the use of the enzyme composition of the invention for the degradation of proteins, for the degradation of by-products, toxic or contaminant proteins; for the degradation of prions or viruses; for the degradation of proteins for proteomics; for the degradation of cornified substrate; for the hydrolysis of polypeptides for amino acid analysis; for wound cleaning; for wound healing; for cosmetology such as peeling tools, depilation, dermabrasion and dermaplaning; for prothesis cleaning and/or preparation; for fabric softeners; for soaps; for tenderizing meat; for the controlled fermentation process of Soja or cheese; for cleaning or disinfection of septic tanks or any container containing proteins that should be removed or sterilized; and for cleaning of surgical instruments. The enzyme composition of the invention can be used in the manufacture of the food supplement of the invention.

Further, the present invention provides a method of degrading a polypeptide substrate, comprising contacting the polypeptide substrate with the enzyme composition of the invention. In the method of degrading a polypeptide substrate, the enzyme composition sequentially digests a full-length polypeptide substrate or a full-length protein. Preferably the polypeptide substrate is selected from the group comprising casein, gluten, bovine serum albumin or fragments thereof and the polypeptide substrate length is from 2 to 200 amino acids.

The present invention also relates a kit for degrading a polypeptide product comprising the enzyme composition of the present invention.

The kit featured herein can also include reagents necessary for carrying out the degradation of a polypeptide product. Said reagents can be buffers, for example sodium citrate buffer, Tris-HCl buffer, and/or acetate buffer; precipitation reagents, such as trichloroacetic acid; and/or the reagents for stopping the enzyme activity, such as acetic acid and/or formic acid. The kit featured herein can further include an information material describing how to perform the degradation of a polypeptide product. The informational material of the kit is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. Of course, the informational material can also be provided in any combination of formats. The kit can also contain separate containers, dividers or compartments for the reagents and informational material. Containers can be appropriately labeled.

The enzyme composition of the invention have numerous applications in food processing industry. For example, the proteases of the invention can be used in the enzymatic treatment of various gluten-containing materials, e.g. from cereals, grains, wine or juice production, or agricultural residues such as vegetable hulls, bean hulls, sugar beet pulp, olive pulp, potato pulp, and the like. The proteases of the invention can be used to modify the consistency and appearance of processed fruit, vegetables or meat. The proteases of the invention can be used to treat plant material to facilitate processing of plant material, including foods, facilitate purification or extraction of plant components.

The enzyme composition according to the present invention can also be added to a food product before its consumption. It can already be added to the food product during production, with the aim that it exhibits its effect only after eating the food product. This could also be achieved by microencapsulation, for example. With this, for example the utilizable proline-rich materials, such as gluten, in the food product would be reduced without negatively affecting its taste. Therefore, preparations containing the enzyme composition according to the present invention are useful, which release the enzyme composition only in the digestive tract of a human (or animal) or let it become effective in another way, especially in the stomach or small intestine. Therefore, the enzyme composition according to the present invention can be used, for example, in the production of desserts, fruit preparations, jam, honey, chocolate and chocolate products, bakery products (e.g. biscuits and cakes), breads, pastas, vegetable dishes, potato dishes, ice cream, cereals, dairy products (e.g. fruit yogurt and pudding), gluten-containing beverages, gluten-containing sauces and gluten-containing sweeteners. For dishes that are boiled or baked, the enzyme composition according to the present invention could, for example, be mixed into or sprinkled onto them after cooling.

The enzyme composition according to the present invention can also be added to a food product, to exert its effect after eating on the gluten originating from another food product. An example of this would be the addition of the enzyme composition according to the present invention to a spread so that the reduction of the gluten that is contained in the bread and that can be used by the body occurs after the intake of the bread, without impairing its taste.

In the modification of food product, the enzyme composition of the present invention can process the food product either in vitro (by modifying components of the food product) or in vivo. The enzyme composition of the invention can be added to food product containing high amounts of gluten, e.g. plant material from cereals, grains and the like. When added to the food product, the enzyme composition of the present invention significantly improves the in vivo break-down of gluten-containing material, e.g., wheat, whereby a better utilization of the plant nutrients by the human (or animal) is achieved.

The enzyme composition according to the present invention may also be used in immobilized form. This is especially useful for the treatment of liquid food products. For example, the enzyme composition of the invention can be embedded in a matrix which is permeable for gluten. If a gluten containing liquid food product is allowed to flow along the enzyme containing matrix, then gluten is extracted from the food product by the action of the enzymes and digested. The enzyme composition of the invention can also be used in the fruit and brewing industry for equipment cleaning and maintenance.

The present invention further provides a method for improving food digestion in a mammal, wherein said method comprising oral administration to the said mammal of the enzyme composition of the invention. Preferably the food contains proline rich nutriments such as gluten and the mammal is a human.

Thus in one aspect, the growth rate and/or food conversion ratio (i.e. the weight of ingested food relative to weight gain) of the human or animal is improved. For example a partially or indigestible proline-comprising protein is fully or partially degraded by the enzyme composition of the invention, resulting in availability of more digestible food for the human or animal. Thus the enzyme composition of the invention of the invention can contribute to the available energy of the food. Also, by contributing to the degradation of proline-comprising proteins, the proteases of the invention can improve the digestibility and uptake of carbohydrate and non-carbohydrate food constituents such as protein, fat and minerals

In one embodiment, the proteases of the enzyme composition of the invention are produced by recombinant DNA techniques. As used herein, the term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques. The person skilled in the art will recognize that these cells can be used for unicellular or multicellular transgenic organisms, for example transgenic fungi producing the enzyme composition of the invention.

Thus the present invention provides a method for producing the enzyme composition of the invention comprising the steps of:
(a) introducing into a host cell a nucleic acid encoding for
   i. a prolyl protease AfuS28 comprising SEQ ID NO: 1, a biologically active fragment thereof, a naturally occurring allelic variant thereof, or a sequence having at least 95% of identity, and
   ii. at least one tripeptidyl protease of the sedolisin family, said tripeptidyl protease selected from the group consisting in
      a) a sedolisin SedA comprising SEQ ID NO: 2, a biologically active fragment thereof, a naturally occurring allelic variant thereof, or a sequence having at least 95% of identity, or
      b) a sedolisin SedB comprising SEQ ID NO: 3, a biologically active fragment thereof, a naturally occurring allelic variant thereof, or a sequence having at least 95% of identity
      c) a sedolisin SedC comprising SEQ ID NO: 4, a biologically active fragment thereof, a naturally occurring allelic variant thereof, or a sequence having at least 95% of identity, or
      d) a sedolisin SedD comprising SEQ ID NO: 5, a biologically active fragment thereof, a naturally occurring allelic variant thereof, or a sequence having at least 95% of identity
(b) cultivating the cell of step (a) in a culture medium under conditions suitable for producing the enzyme composition; and
(c) recovering the enzyme composition.

Optionally, one or more nucleic acids encoding proteases selected from the group comprising:
an aspartic protease of the pepsin family (Pep1) comprising SEQ ID NO: 6, a biologically active fragment thereof, a naturally occurring allelic variant thereof, or a sequence having at least 95% of identity.
a glutamic protease serine comprising SEQ ID NO: 7, a biologically active fragment thereof, a naturally occurring allelic variant thereof, or a sequence having at least 95% of identity.
carboxypeptidase Scp1 comprising SEQ ID NO:8, a biologically active fragment thereof, a naturally occurring allelic variant thereof, or a sequence having at least 95% of identity, and
X-prolyl peptidase (DppIV) comprising SEQ ID NO:9, a biologically active fragment thereof, a naturally occurring allelic variant thereof, or a sequence having at least 95% of identity.
   can be additionally introduced into the host cell.

Preferably, the additional nucleic acid encodes X-prolyl peptidase (DppIV) comprising SEQ ID NO:9, a biologically active fragment thereof, a naturally occurring allelic variant thereof, or a sequence having at least 70% of identity.

(AfuS28)
SEQ ID NO: 10
atgcggactg ctgctgcttc actgacgctt gctgcgactt gtctctttga gttggcatct
gctctcatgc ccagggcgcc tttgatccct gcgatgaaag cgaaagttgc cttgccctct
ggaaacgcga cattcgagca gtatattgat cataataacc ccggtctggg aacatttccc
cagagatact ggtataatcc ggagttttgg gccggtcctg gctctcctgt gcttttgttt
acaccgggtg aatcagatgc tgcggactac gacggattcc tgaccaacaa gacgattgtt
ggacgctttg ccgaagagat cgggggcgcg gttatcctgc ttgagcatcg ctactgggga
gcctcatcac cttatcccga gttgaccacc gagacgctcc agtacctgac tctggagcag
tcgatcgcag accttgttca ctttgcaaag actgtgaatc ttccgttcga cgagattcac
agcagcaacg ccgataacgc gccatgggtg atgactgggg gatcctacag tggtgctcta
gccgcgtgga ccgcatcaat tgctccaggg accttctggg cgtaccatgc atcgagtgca
ccggtgcagg ccatctatga cttctggcaa tatttcgtcc ccgttgtcga ggggatgccc
aagaactgca gcaaggatct caaccgcgtg gtggagtata ttgaccacgt ctatgagtcg
ggggatatcg agcgccagca ggaaatcaaa gagatgttcg ggttgggagc tctcaagcat
tttgacgatt ttgcagcagc aattacgaac ggaccatggc tttggcagga tatgaatttc
gtctcggggt actcccgttt ttataaattt tgcgatgcgg tagagaatgt cactccgggg
gcaaagtccg ttcctggacc ggaaggcgtc ggtctggaga aagcactcca aggctatgcg
tcatggttca attcaacgta cttgcctggc tcttgcgccg aatacaaata ttggaccgac
aaagacgcag ttgactgtta cgactcttat gagactaaca gccccattta caccgacaag
gccgtcaaca atacctccaa taagcagtgg acctggttct tatgcaatga acctctcttc
tactggcaag atggtgcacc caaggatgag tccaccattg tctccagaat cgtctcagca
gagtactggc agcgacaatg tcacgcgtat ttcccagaag tcaacggcta tacgttcggt
agcgccaatg gcaagaccgc tgaagacgtg aataagtgga ccaagggctg ggacttgacc
aacacaacac gtctgatctg ggcaaatggt caattcgatc cctggaggga cgcctcagtt
tcctccaaaa cgagacccgg aggacccctt cagtccacag aacaagcgcc agtacatgta
attccgggtg ggttccattg ctcagatcaa tggctagtct atggggaggc gaatgccggc
gttcaaaagg tgattgatga agaagtggcg caaatcaagg cttgggtcgc ggagtatccc
aaatatagga agccatga
(SedA)
SEQ ID NO: 11
ATGCGACTTTCACACGTACTCCTAGGAACTGCAGCTGCAGCTGGCGTTCTGGCTA
GTCCCACCCCGAACGACTATGTCGTGCATGAACGTCGTGCTGTCCTCCCTCGCTC
CTGGACGGAGGAGAAGAGACTTGATAAGGCCTCTATCTTGCCTATGAGGATTGG
TCTCACTCAGTCTAACCTAGATCGCGGTCATGACTTGTTGATGGAGATATCTGAT
CCGCGCTCGTCACGCTATGGACAACATCTCTCCGTCGAGGAGGTCCACAGTCTCT
TTGCTCCGAGCCAGGAGACTGTCGACCGTGTTCGAGCATGGCTTGAGTCTGAGGG
CATAGCCGGCGACCGCATCTCTCAGTCCTCGAACGAGCAATTCCTGCAATTTGAC
GCGAGTGCGGCGGAAGTTGAAAGGCTATTGGGTACTGAGTACTATCTCTATACA
CATCAAGGTTCAGGAAAGTCACACATTGCTTGCCGAGAATACCATGTCCCCCACT
CATTGCAGCGGCATATCGACTACATTACCCCTGGCATCAAGCTCCTAGAGGTGGA
AGGAGTCAAGAAAGCTCGGAGCATTGAAAAGCGTTCATTCAGAAGCCCGCTGCC
GCCAATCCTTGAGCGGCTTACCCTTCCCTTGTCCGAGCTGCTGGGTAATACTTTAT
TGTGTGATGTGGCCATAACACCACTGTGTATATCAGCTCTCTACAACATTACTCG
CGGCTCAAAAGCTACCAAGGGCAATGAACTGGGCATCTTTGAGGATCTAGGGGA
TGTTTACAGTCAAGAGGATCTCAACCTGTTCTTTTCAACATTTGCACAGCAAATT
CCCCAGGGCACTCATCCCATCCTGAAGGCCGTCGACGGCGCTCAAGCCCCAACC
AGCGTGACCAATGCAGGGCCCGAATCCGACCTGGACTTTCAAATCTCGTATCCGA
TCATCTGGCCGCAGAACTCCATTCTCTTTCAAACAGATGATCCAAATTACACAGC
AAACTACAACTTCAGTGGCTTTTTGAACACCTTTTTGGATGCTATCGATGGATCCT
ACTGCAGCGAGATCTCCCCTCTGGACCCGCCGTACCCCAATCCCGCCGACGGCGG
CTACAAAGGCCAACTCCAGTGCGGCGTCTACCAGCCCCCCAAGGTTCTCTCCATC
TCGTACGGCGGCGCCGAGGCCGACCTCCCCATCGCGTACCAGCGCCGCCAGTGC
GCCGAGTGGATGAAACTCGGCCTGCAGGGTGTCTCCGTCGTCGTCGCATCCGGCG
ACTCCGGCGTCGAAGGCAGGAATGGCGATCCCACCCCCACTGAGTGCCTCGGGA
CGGAAGGGAAAGTCTTCGCCCCGGACTTCCCGGCCACCTGTCCCTACCTCACCAC
CGTCGGCGGGACCTACCTCCCCCTCGGCGCCGACCCCCGCAAGGACGAAGAAGT
CGCCGTGACCTCGTTCCCCTCGGGCGGCGGGTTCAGCAACATCTACGAGCGCGCA
GACTACCAGCAGCAAGCCGTCGAGGACTACTTCTCCCGCGCCGATCCCGGGTAC
CCGTTCTACGAGAGCGTCGACAACAGCAGCTTCGCGGAGAACGGCGGCATCTAC
AACCGGATTGGGCGCGCGTACCCGGACGTCGCAGCCATCGCGGACAACGTCGTG
ATCTTCAACAAGGGCATGCCGACGCTTATTGGCGGTACCTCGGCTGCTGCGCCGG
TGTTTGCAGCCATCCTGACTAGGATTAACGAGGAGCGGCTCGCGGTCGGCAAGT
CGACCGTGGGATTTGTGAACCCCGTGCTGTATGCGCATCCCGAGGTGTTTAATGA
TATCACGCAGGGGAGTAACCCGGGCTGTGGCATGCAAGGGTTCTCCGCTGCGAC
GGGATGGGATCCGGTGACGGGGTTGGGAACTCCGAATTATCCAGCACTTTTAGA
CTTGTTCATGAGCCTGCCGTAG
(SedB)
SEQ ID NO: 12
ATGTTTTCGTCGCTCTTGAACCGTGGAGCTTTGCTCGCGGTTGTTTCTCTCTTGTC
CTCTTCCGTTGCTGCCGAGGTTTTTGAGAAGCTGTCCGCGGTGCCACAGGGATGG
AAATACTCCCACACCCCTAGTGACCGCGATCCCATTCGCCTCCAGATTGCCCTGA
AGCAACATGATGTCGAAGGTTTTGAGACCGCCCTCCTGGAAATGTCCGATCCCTA
CCACCCAAACTATGGCAAGCACTTTCAAACTCACGAGGAGATGAAGCGGATGCT
GCTGCCCACCCAGGAGGCGGTCGAGTCCGTCCGCGGCTGGCTGGAGTCCGCTGG
AATCTCGGATATCGAGGAGGATGCAGACTGGATCAAGTTCCGCACAACCGTTGG
CGTGGCCAATGACCTGCTGGACGCCGACTTCAAGTGGTACGTGAACGAGGTGGG
CCACGTTGAGCGCCTGAGGACCCTGGCATACTCGCTCCCGCAGTCGGTCGCGTCG
CACGTCAACATGGTCCAGCCCACCACGCGGTTCGGACAGATCAAGCCCAACCGG
GCGACCATGCGCGGTCGGCCCGTGCAGGTGGATGCGGACATCCTGTCCGCGGCC
GTTCAAGCCGGCGACACCTCCACTTGCGATCAGGTCATCACCCCTCAGTGCCTCA
AGGATCTGTACAATATCGGCGACTACAAGGCCGACCCCAACGGGGGCAGCAAGG
TCGCGTTTGCCAGTTTCCTGGAGGAATACGCCCGCTACGACGATCTGGCCAAGTT
CGAGGAGAAGCTGGCCCCGTACGCCATTGGACAGAACTTTAGCGTGATCCAGTA
CAACGGCGGTCTGAACGACCAGAACTCCGCCAGTGACAGCGGGGAGGCCAATCT
CGACCTGCAGTACATCGTTGGTGTCAGCTCGCCCATTCCGGTCACCGAGTTCAGC
ACCGGTGGCCGGGGTCTTCTCATTCCGGACCTGAGCCAGCCCGACCCCAACGAC
AACAGCAACGAGCCGTATCTGGAATTCCTGCAGAATGTGTTGAAGATGGACCAG
GATAAGCTCCCTCAGGTCATCTCCACCTCCTATGGCGAGGATGAACAGACCATTC
CCGAAAAATACGCGCGCTCGGTCTGCAACCTGTACGCTCAGCTGGGCAGCCGCG
GGGTTTCGGTCATTTTCTCCTCTGGTGACTCCGGTGTTGGCGCGGCTTGCTTGACC
AACGACGGCACCAACCGCACGCACTTCCCCCCACAGTTCCCTGCGGCCTGCCCCT
GGGTGACCTCGGTGGGTGGCACGACCAAGACCCAGCCCGAGGAGGCGGTGTACT
TTTCGTCGGGCGGTTTCTCCGACCTGTGGGAGCGCCCTTCCTGGCAGGATTCGGC
GGTCAAGCGCTATCTCAAGAAGCTGGGCCCTCGGTACAAGGGCCTGTACAACCC
CAAGGGCCGTGCCTTCCCCGATGTTGCTGCCCAGGCCGAGAACTACGCCGTGTTC
GACAAGGGGGTGCTGCACCAGTTTGACGGAACCTCGTGCTCGGCTCCCGCATTTA
GCGCTATCGTCGCATTGCTGAACGATGCGCGTCTGCGCGCTCACAAGCCCGTCAT
GGGTTTCCTGAACCCCTGGCTGTATAGCAAGGCCAGCAAGGGTTTCAACGATATC
GTCAAGGGCGGTAGCAAGGGCTGCGACGGTCGCAACCGATTCGGAGGTACTCCC
AATGGCAGCCCTGTGGTGCCCTATGCCAGCTGGAATGCCACTGACGGCTGGGAC
CCGGCCACGGGTCTAGGGACTCCGGACTTTGGCAAGCTTCTGTCTCTTGCTATGC
GGAGATAG
(SedC)
SEQ ID NO: 13
ATGGCTCCATTCACGTTTCTGGTAGGGATACTATCCCTCTGTATTTGCTGCATTGT
TCTTGGTGCAGCTGCAGAGCCCAGCTACGCGGTCGTTGAGCAGCTCAGAAATGTT
CCCGACGGCTGGATAAAGCACGATGCAGCGCCAGCGTCTGAATTGATCAGATTT
CGGCTGGCTATGAACCAGGAAAGAGCCGCTGAATTCGAGCGAAGGGTCATTGAC
ATGTCAACGCCGGGTCACTCGAGCTATGGACAACATATGAAGCGTGACGATGTC
AGGGAATTTCTGCGTCCTCCCGAGGAGGTTTCAGACAAAGTCCTTTCCTGGCTGA
GATCAGAGAATGTTCCTGCTGGCTCGATTGAAAGTCATGGCAACTGGGTCACTTT
CACTGTCCCGGTATCACAGGCGGAACGTATGCTAAGAACACGCTTTTACGCCTTC
CAGCACGTGGAGACAAGTACGACACAAGTCAGAACGCTTGCGTATTCCGTTCCA
CATGACGTCCACCGCTATATTCAGATGATCCAGCCAACGACTCGCTTTGGACAAC
CTGCCCGGCATGAACGGCAACCACTTTTCCACGGGACTGTTGCTACCAAGGAAG
AGCTGGCGGCGAATTGCTCCACAACCATAACGCCGAACTGCCTTCGCGAATTGTA
CGGGATTTATGATACCAGAGCCGAACCCGATCCCCGCAACAGACTGGGAGTTTC
CGGGTTCCTAGATCAGTACGCACGTTACGACGACTTTGAAAATTTTATGAGATTG
TATGCAACCAGTAGGACAGACGTCAACTTCACTGTGGTCTCGATAAATGACGGTC
TCAATCTGCAGGACTCGTCCCTGAGCAGTACCGAAGCCAGCCTAGACGTCCAGT
ATGCCTATTCTTTGGCGTATAAAGCGCTTGGAACCTACTATACAACGGGTGGCCG
AGGACCGGTTGTGCCTGAGGAAGGTCAGGATACGAACGTGTCGACCAATGAGCC
TTACTTAGATCAACTTCATTATCTTCTTGATCTTCCAGATGAAGAGCTTCCCGCCG
TTCTTTCAACCTCGTATGGTGAAGATGAGCAAAGCGTCCCTGAATCATACTCAAA
TGCAACATGCAATCTGTTCGCGCAGCTTGGCGCACGCGGCGTGTCGATCATCTTC
AGCAGCGGTGACTCAGGCGTTGGTTCAACATGCATAACTAACGATGGAACCAAG
ACAACTCGATTCTTGCCTGTCTTCCCAGCGTCCTGCCCATTTGTTACTGCTGTCGG
CGGTACTCACGATATCCAACCCGAGAAAGCAATTAGCTTCTCTAGCGGAGGCTTT
TCAGATCACTTTCCACGTCCCTCCTATCAGGATTCAAGCGTTCAAGGCTACCTAG
AGCAGCTTGGAAGCAGATGGAACGGGTTATACAACCCGAGCGGGAGAGGTTTCC
CTGACGTCGCCGCTCAGGCCACTAACTTTGTCGTCATTGATCACGGGCAAACGTT
GAGGGTAGGCGGCACAAGTGCATCTGCGCCTGTATTTGCAGCCATAGTCTCGCG
ATTAAATGCTGCTCGACTTGAGGATGGTTTGCTAAAACTGGGGTTCTTAAATCCA
TGGCTCTATTCCCTCAACCAGACAGGATTCACAGACATTATTGATGGTGGCTCAT
CGGGTTGCTATGTTGGCACCAGCAACGAGCAACTGGTTCCCAATGCAAGCTGGA
ATGCAACGCCAGGATGGGATCCTGTTACCGGGCTTGGGACGCCCATTTATAATAC
CCTGGTGAAATTGGCCACGAGTGTTTCAAGTACCCCATGA
(SedD)
SEQ ID NO: 14
ATGCTGTCCTCGACTCTCTACGCAGGGTGGCTCCTCTCCCTCGCAGCCCCAGCCC
TTTGTGTGGTGCAGGAGAAGCTCTCAGCTGTTCCTAGTGGCTGGACACTCATCGA
GGATGCATCGGAGAGCGACACGATCACTCTCTCAATTGCCCTTGCTCGGCAGAAC
CTCGACCAGCTTGAGTCCAAGCTGACCACGCTGGCGACCCCAGGGAACCCGGAG
TACGGCAAGTGGCTGGACCAGTCCGACATTGAGTCCCTATTTCCTACTGCAAGCG
ATGATGCTGTTCTCCAATGGCTCAAGGCGGCCGGGATTACCCAAGTGTCTCGTCA
GGGCAGCTTGGTGAACTTCGCCACCACTGTGGGAACAGCGAACAAGCTCTTTGA
CACCAAGTTCTCTTACTACCGCAATGGTGCTTCCCAGAAACTGCGTACCACGCAG
TACTCCATCCCCGATCACCTGACAGAGTCGATCGATCTGATTGCCCCCACTGTCT
TCTTTGGCAAGGAGCAGAACAGCGCACTGTCATCTCACGCAGTGAAGCTTCCAG
CTCTTCCTAGGAGGGCAGCCACCAACAGTTCTTGCGCCAACCTGATCACCCCCGA
CTGCCTAGTGGAGATGTACAACCTCGGCGACTACAAACCTGATGCATCTTCGGGA
AGTCGAGTCGGCTTCGGTAGCTTCTTGAATGAGTCGGCCAACTATGCAGATTTGG
CTGCGTATGAGCAACTCTTCAACATCCCACCCCAGAATTTCTCAGTCGAATTGAT
CAACAGAGGCGTCAATGATCAGAATTGGGCCACTGCTTCCCTCGGCGAGGCCAA
TCTGGACGTGGAGTTGATTGTAGCCGTCAGCCACCCCCTGCCAGTAGTGGAGTTT
ATCACTGGCGCCCTACCTCCAGTACTACGAGTACTTGCTCTCCAAACCCAACTCC
CATCTTCCTCAGGTGATTTCCAACTCACTGTTCCCGAGTACTACGCCAGGAGAGT
TTGCAACTTGATCGGCTTGATGGGTCTTCGTGGCATCACGGTGCTCGAGTCCTCT
GGTGATACCGGAATCGGCTCGGCATGCATGTCCAATGACGGCACCAACAAGCCC
CAATTCACTCCTACATTCCCTGGCACCTGCCCCTTCATCACCGCAGTTGGTGGTAC
TCAGTCCTATGCTCCTGAAGTTGCTTGGGACGGCAGTTCCGGCGGATTCAGCAAC
TACTTCAGCCGTCCCTGGTACCAGTCTTTCGCGGTGGACAACTACCTCAACAACC
ACATTACCAAGGATACCAAGAAGTACTATTCGCAGTACACCAACTTCAAGGGCC
GTGGATTCCCTGATGTTTCCGCCCATAGTTTGACCCCTTACTACGAGGTCGTCTTG
ACTGGCAAACACTACAAGTCTGGCGGCACATCCGCCGCCAGCCCCGTCTTTGCCG
GTATTGTCGGTCTGCTGAACGACGCCCGTCTGCGCGCCGGCAAGTCCACTCTTGG
CTTCCTGAACCCATTGCTGTATAGCATCCTGGCCGAAGGATTCACCGATATCACT
GCCGGAAGTTCAATCGGTTGTAATGGTATCAACCCACAGACCGGAAAGCCAGTT
CCTGGTGGTGGTATTATCCCCTACGCTCACTGGAACGCTACTGCCGGCTGGGATC
CTGTTACTGGCCTTGGGGTTCCTGATTTCATGAAATTGAAGGAGTTGGTTCTGTC
GTTGTAA
(Pep1)
SEQ ID NO: 15
ATGGTCGTCTTTAGCAAAGTCACCGCTGTCGTCGTCGGTCTCTCGACCATTGTGTCTG
CTGTCCCTGTGGTCCAGCCGCGCAAGGGCTTCACTATCAACCAAGTGGCCAGACCAG
TGACCAACAAGAAGACCGTCAATCTTCCAGCTGTCTATGCCAATGCTTTGACTAAGT
ACGGGGGCACTGTCCCCGACAGTGTCAAGGCGGCTGCAAGCTCCGGCAGCGCTGTT
ACTACCCCCGAGCAATATGACTCGGAATACCTGACCCCCGTCAAAGTCGGTGGAAC
GACCCTGAACTTGGACTTCGACACTGGCTCTGCAGATCTCTGGGTCTTCTCCTCCGA
GCTTTCGGCTTCCCAGTCCAGCGGCCATGCTATCTACAAGCCGTCCGCTAATGCCCA
AAAGCTGAATGGCTACACCTGGAAGATCCAATATGGTGATGGTAGCAGTGCCAGCG
GTGACGTCTACAAGGATACCGTCACTGTGGGTGGTGTCACTGCTCAGAGCCAGGCTG
TGGAGGCTGCCAGCCATATCAGCTCTCAATTCGTGCAGGATAAGGACAACGATGGT
CTGTTGGGTTTGGCATTCAGCTCCATCAACACTGTCAGTCCCCGCCCTCAGACTACTT
TCTTTGACACTGTCAAGTCCCAGTTGGACTCTCCTCTCTTTGCTGTGACCTTGAAGTA
CCATGCTCCAGGCACCTACGACTTTGGATACATCGACAACTCCAAGTTCCAAGGGGA
ACTCACTTATACCGACGTCGACAGCTCCCAGGGTTTCTGGATGTTCACTGCTGATGG
CTACGGTGTTGGCAATGGTGCTCCCAACTCCAACAGTATCAGCGGCATTGCTGACAC
CGGCACCACCCTCCTCCTGCTTGATGACAGCGTTGTTGCCGACTACTACCGCCAGGT
TTCCGGAGCCAAGAACAGCAACCAATACGGTGGTTATGTCTTCCCCTGCTCCACCAA
ACTTCCTTCTTTCACTACCGTCATCGGAGGCTACAATGCCGTCGTTCCCGGTGAATAC
ATCAACTACGCCCCCGTCACTGACGGCAGCTCTACCTGCTACGGCGGCATCCAGAGC
AACTCTGGTTTGGGCTTTTCTATCTTCGGAGATATCTTCCTCAAGAGCCAGTACGTCG
TCTTCGACTCCCAAGGCCCCAGACTCGGCTTCGCCCCTCAGGCATAG
(Glutamic protease)
SEQ ID NO: 16
ATGAAGTTCACTTCTGTCCTCGCCTCCGGCTTGCTTGCCACGGCTGCCATCGCTGC
TCCCCTCACAGAACAGCGTCAAGCCCGGCATGCCCGTCGTCTGGCCCGCACCGCC
AACAGATCGAGCCACCCTCCCTACAAGCCCGGCACTTCCGAGGTTATCAAGCTCA
GCAACACCACCCAGGTCGAGTACAGCTCCAACTGGGCTGGTGCCGTCCTCATCG
GCACAGGCTACACGGCTGTGACTGGCGAGTTCGTCGTCCCTACCCCCAGCGTCCC
AAGCGGTGGCTCTTCCAGCAAGCAGTACTGCGCCTCCGCTTGGGTCGGTATCGAC
GGTGACACCTGCAGCTCTGCCATCCTGCAAACCGGCGTCGACTTCTGCATCCAGG
GCAGCTCTGTCTCCTTCGACGCCTGGTACGAGTGGTACCCCGACTACGCGTACGA
CTTCAGCGGCATCTCCATCTCCGCTGGCGACACGATCAGGGTCACCGTTGATGCA
ACCAGCAAGACCGCTGGCACGGCCACTGTCGAGAATGTGACCAAGGGCAAGACT
GTCACCCACACCTTCACCGGCGGCGTGGACGGCAATCTGTGCGAGTACAATGCC
GAGTGGATCGTTGAAGACTTTGAGTCCAACGGGTCTCTGGTGCCGTTTGCTAACT
TTGGCACTGTCACCTTCACCGGGGCTCAGGCTACCGATGGCGGTTCCACTGTTGG
GCCTTCTGGCGCCACTCTGATTGATATCCAGCAGAGCGGCAAGGTTTTGACTTCG
GTTTCTACCTCTAGCAGCTCTGTCACTGTTAAGTATGTCTAA
(Scp1)
SEQ ID NO: 17
ATGCTATCCCTCGTAACCCTTCTATCTGGGACCGCTGGTCTTGCATTGACCGCGTC
GGCACAGTATTTCCCTCCCACTCCCGAGGGTCTCAAGGTCGTGCATTCGAAGCAC
CAGGAGGGCGTGAAGATTTCGTACAAAGAACCTGGTATTTGTGAAACCACCCCG
GGTGTCAAATCGTACTCCGGCTATGTACATCTGCCGCCCGGCACGCTGAACGACG
TTGATGTCGACCAGCAATACCCCATCAACACTTTCTTCTGCTTCTTCGAGTCGCGC
AATGATCCCATTCACGCACCGCTGGCCATTTGGATGAACGGCGGTCCCGGCAGCT
CGTCCATGATCGGACTACTGCAGGAAAATGGCCCGTGTCTTGTAAACGCCGACTC
CAACTCAACGGAGATCAACCCCTGGTCGTGGAACAACTACGTCAACATGCTGTA
CATTGATCAGCCGAACCAGGTTGGGTTCAGCTACGATGTTCCTACAAACGGGAC
GTATAACCAGCTCACCACTGCGTGGAATGTGTCTGCATTCCCGGATGGTAAAGTC
CCGGAGCAGAACAATACATTCTATGTGGGCACGTTCCCCAGTATGAACCGGACG
GCTACGGCAAATACGACGCAGAATGCGGCGCGGTCGCTTTGGCACTTTGCGCAG
ACGTGGTTCTCTGAATTCCCCGAGTACAAGCCGCACGATGACCGGGTGAGTATCT
GGACTGAGTCATATGGTGGTCGATACGGGCCGTCGTTCGCGGCGTTCTTTCAGGA
ACAGAATGAGAAGATCGAAGAGGGGGCGTTACCAGATGAGTACCATTACATTCA
CCTGGACACTCTGGGAATCATCAATGGGTGCGTGGATTTGTTGACCCAAGCGCCG
TTCTACCCGGATATGGCGTACAACAATACCTACGGCATCGAGGCGATCAACAAG
ACCGTCTACGAAAGGGCAATGAATGCGTGGAGTAAGCCCGGTGGCTGCAAGGAC
CTGATAGTCAAGTGCCGTGAGCTAGCGGCCGAGGGAGATCCAACCATGTCCGGc
CACAACGAGACGGTCAACGAGGCCTGTCGAAGGGCGAACGACTACTGCAGCAAC
CAGGTGGAAGGCCCCTACATACTGTTCTCCAAGCGTGGCTACTACGATATCGCGC
ACTTTGATCCAGATCCATTTCCACCACCTTATTTCCAAGGTTTCCTGAACCAGAAC
TGGGTACAAGCCGCCCTGGGGGTGCCCGTCAACTTCTCCATCTCAGTGGACAGCA
CATACAGCGCCTTTGCGTCGACGGGCGACTATCCGCGCGCCGATGTTCACGGGTA
CCTCGAGGATCTTGCATATGTCCTCGACTCGGGGATCAAAGTGGCGCTCGTCTAC
GGAGACCGGGACTACGCATGTCCCTGGAACGGCGGCGAAGAGGTTAGTTTGCGC
GTCAACTATTCCGACTCGCAGTCGTTCCAAAAAGCAGGCTACGCCCCGGTCCAGA
CCAATTCGTCATATATCGGGGGCCGGGTGCGGCAGTACGGCAACTTTTCTTTCAC
GCGTGTCTTCGAAGCGGGCCATGAGGTGCCAGCGTATCAACCGCAGACGGCCTA
TGAGATCTTCCACAGAGCGTTATTTAATCGAGACATTGCGACGGGGAAGATGTC
ACTACTGAAGAATGCCACCTACGCGAGCGAGGGCCCATCCTCGACGTGGGAATT
TAAGAATGAGGTACCTGAGAGTCCGGAGCCGACCTGTTATATCCAGTCATTGCA
GAGTAGTTGCACCGAAGAGCAGATCCAGAGCGTGGTCAACGGCACTGCTTTGAT
TAAAGATTGGATCGTGGTGGAGAAAGTGGACATTTACTAG
(DppIV)
SEQ ID NO: 18
ATGAAGTGGTCAATTCTCCTTTTGGTCGGCTGCGCTGCCGCCATTGACGTCCCTC
GTCAACCATATGCCCCTACTGGAAGCGGCAAGAAACGACTGACCTTCAACGAGA
CGGTCGTCAAGCGAGCCATTTCCCCCTCGGCCATCTCGGTCGAGTGGATTTCTAC
CTCCGAGGATGGGGATTATGTCTACCAAGACCAGGACGGCAGTCTGAAAATCCA
GAGCATCGTCACCAACCACACGCAGACCCTCGTCCCTGCGGACAAAGTGCCAGA
GGATGCCTACAGCTACTGGATCCATCCCAATCTCTCCTCCGTGCTCTGGGCTACC
AACTACACCAAGCAATACCGGCACTCGTACTTTGCCGACTACTTTATCCAGGACG
TGCAGTCGATGAAATTGCGACCGCTCGCCCCAGACCAGTCCGGCGACATCCAGT
ACGCTCAGTGGACTCCCACCGGCGACGCCATCGCCTTTGTCCGCGACAACAACGT
CTTCGTCTGGACCAATGCCTCGACTAGCCAGATTACCAATGACGGCGGGCCGGAT
CTCTTCAATGGCGTCCCGGACTGGATCTACGAGGAGGAGATCCTCGGCGACCGG
TTTGCGCTCTGGTTCTCGCCGGACGGGGCGTACCTCGCCTTCCTGCGGTTCAATG
AGACCGGTGTCCCAACCTTCACCGTGCCGTACTACATGGACAACGAGGAGATTG
CGCCGCCGTACCCACGCGAGCTGGAGCTGCGGTATCCCAAGGTGTCGCAGACGA
ACCCTACCGTCGAGCTGAACCTGCTGGAGCTCCGTACCGGCGAGCGGACGCCTG
TCCCGATCGACGCCTTTGACGCAAAGGAGCTGATCATCGGCGAGGTGGCGTGGT
TGACGGGGAAGCATGACGTCGTGGCTGTCAAGGCGTTCAACCGCGTGCAGGACC
GGCAAAAGGTCGTCGCTGTGGATGTGGCCTCGCTCAGGTCCAAGACAATTAGTG
AGCGCGACGGCACGGACGGATGGCTGGATAACCTGCTCTCCATGGCGTACATCG
GGCCCATCGGCGAGTCCAAGGAGGAGTACTACATTGACATCTCGGACCAGTCCG
GCTGGGCGCATCTCTGGCTGTTTCCTGTCGCCGGAGGCGAGCCCATCGCCCTGAC
CAAGGGCGAGTGGGAAGTCACCAATATCCTTAGCATCGACAAGCCGCGCCAGCT
GGTCTACTTCCTGTCGACCAAACACCACAGCACCGAGCGCCACCTCTACTCCGTC
TCCTGGAAGACGAAAGAAATCACCCCCTTAGTCGACGACACCGTCCCCGCCGTCT
GGTCCGCCTCCTTCTCCTCGCAGGGCGGATACTACATCCTCTCTTACCGCGGGCC
CGACGTGCCCTACCAAGACCTCTACGCCATCAACTCCACCGCGCCCCTGCGCACC
ATCACCAGCAACGCGGCCGTGCTCAACGCCTTGAAGGAATACACCTTGCCGAAC
ATTACCTACTTCGAGCTCGCCCTTCCCAGCGGCGAAACCCTCAACGTCATGCAGC
GCCTCCCCGTCAAGTTCTCCCCCAAGAAGAAGTACCCCGTTCTCTTCACCCCCTA
CGGCGGTCCCGGCGCACAAGAAGTCTCCAAAGCCTGGCAAGCCCTCGACTTCAA
GGCCTACATTGCCTCAGACCCCGAACTCGAGTATATCACCTGGACGGTTGACAAC
CGCGGCACGGGCTACAAGGGCCGCGCATTCCGGTGCCAAGTTGCCAGCCGGCTG
GGCGAGCTCGAAGCCGCCGACCAGGTCTTCGCCGCGCAGCAGGCCGCCAAGCTC
CCTTATGTCGACGCACAGCACATCGCCATATGGGGATGGAGTTACGGCGGCTATC
TGACGGGCAAGGTCATCGAGACCGACAGTGGGGCGTTCTCGCTTGGTGTGCAGA
CCGCTCCGGTTTCGGACTGGCGATTCTATGATTCGATGTACACGGAGCGGTATAT
GAAGACGCTGGAGAGCAACGCGGCAGGGTACAATGCCAGTGCGATCCGGAAGG
TAGCAGGCTACAAGAATGTGCGTGGTGGGGTGCTGATCCAGCATGGGACGGGTG
ACGATAATGTGCATTTCCAGAATGCGGCGGCGCTGGTGGACACCCTTGTTGGGGC
GGGAGTGACACCGGAGAAGCTGCAGGTGCAGTGGTTTACAGACTCGGATCATGG
GATTCGGTACCATGGGGGGAATGTGTTCTTGTATCGGCAGTTGTCCAAGAGGCTG
TACGAGGAGAAGAAGCGGAAGGAGAAGGGTGAGGCGCATCAGTGGAGCAAGAA
GTCTGTTCTGTAG

The nucleic acids encoding the proteases of the enzyme composition of the invention include the nucleic acids whose sequences are provided herein or fragments thereof. The invention also includes mutant or variant nucleic acids any of whose bases may be changed from the corresponding base shown herein, while still encoding a protease that maintains activities of the proteases of the invention, or a fragment of such a nucleic acid. The invention further includes nucleic acids whose sequences are complementary to those described herein, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

Also included in the invention are fragments of nucleic acids sufficient for use as hybridization probes to identify protease-encoding nucleic acids (for example AfuS28 mRNAs) and fragments for use as PCR primers for the amplification and/or mutation of protease nucleic acid molecules.

A nucleic acid molecule of the invention, e.g., a nucleic acid molecule having the nucleic acid sequence comprising SEQ ID NOs: 10, 11, 12, 13, 14, 15, 16, 17 or 18, a complement of this aforementioned nucleic acid sequence, can be isolated using standard molecular biology techniques and the sequence information provided herein. Using all or a portion of the nucleic acid sequence of SEQ ID NOs: 10, 11, 12, 13, 14, 15, 16, 17 or 18 as a hybridization probe, nucleic acid molecules can be isolated using standard hybridization and cloning techniques (e.g., as described in Sambrook et al., (eds.), MOLECULAR CLONING: A LABORATORY MANUAL 2^{nd} Ed., Cold Spring Harbor Laboratory Press, Cold Spring Harbor, N.Y., 1989; and Ausubel et al., (eds.), CURRENT PROTOCOLS IN MOLECULAR BIOLOGY, John Wiley & Sons, New York, N.Y., 1993.)

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded.

The term “probes”, as used herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules, which are present in the natural source of these nucleic acid molecules. Preferably, an “isolated” nucleic acid is free of sequences, which naturally flank the nucleic acid (e.g., sequences located at the 5′- and 3′-termini of the nucleic acid) in the genomic DNA of the organism from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized. Particularly, it means that the nucleic acid or protein is at least about 50% pure, more preferably at least about 85% pure, and most preferably at least about 99% pure.

A nucleic acid molecule of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to protease nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. Oligonucleotides may be chemically synthesized and may also be used as probes.

In another embodiment, an isolated nucleic acid molecule of the invention comprises a nucleic acid molecule that is a complement of the nucleic acid sequence shown in SEQ ID NOs: 10, 11, 12, 13, 14, 15, 16, 17 or 18, or a portion of this nucleic acid sequence (e.g., a fragment that can be used as a probe or primer or a fragment encoding a biologically-active fragment of a protease of the invention). A nucleic acid molecule that is complementary to the nucleic acid sequence shown in SEQ ID NOs: 10, 11, 12, 13, 14, 15, 16, 17 or 18 is one that is sufficiently complementary to the nucleic acid sequence shown in SEQ ID NOs: 10, 11, 12, 13, 14, 15, 16, 17 or 18 that it can hydrogen bond with little or no mismatches to the nucleic acid sequence shown in SEQ ID NOs: 10, 11, 12, 13, 14, 15, 16, 17 or 18, thereby forming a stable duplex.

As used herein, the term “complementary” refers to Watson-Crick or Hoogsteen base pairing between nucleotide units of a nucleic acid molecule.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differ from it with respect to certain components or side chains. Analogs may be synthetic or from a different evolutionary origin and may have a similar or opposite metabolic activity compared to wild type. Homologs or orthologs are nucleic acid sequences or amino acid sequences of a particular gene that are derived from different species.

Derivatives or analogs of the nucleic acids or proteins of the invention include, but are not limited to, molecules comprising regions that are substantially homologous to the nucleic acids or proteins of the invention, in various embodiments, by at least about 70%, 80%, 90% or 95% identity over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See, e.g., Ausubel et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level. Homologous nucleotide sequences encode those sequences coding for isoforms of proteases of the invention. Isoforms can be expressed in the same organism as a result of, for example, alternative splicing of RNA. Alternatively, iso forms can be encoded by different genes. In the invention, homologous nucleotide sequences can include nucleotide sequences encoding a protease of the invention of species other than fungi. Homologous nucleotide sequences also include, but are not limited to, naturally occurring allelic variations and mutations of the nucleotide sequences set forth herein. Homologous nucleic acid sequences include those nucleic acid sequences that encode conservative amino acid substitutions in SEQ ID NOs: 1, 2, 3, 4, 5, 6, 7, 8 or 9, as well as a polypeptide possessing biological activity of the protease of the invention.

The nucleic acid sequence identity may be determined as the degree of identity between two sequences. The identity may be determined using computer programs known in the art, such as GAP software provided in the GCG program package. See Needleman & Wunsch, J. Mol. Biol. 48:443-453 1970. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the nucleic acid sequence shown in SEQ ID NOs: 10, 11, 12, 13, 14, 15, 16, 17 or 18.

A protease of the invention is encoded by the open reading frame (“ORF”) of a nucleic acid of said protease. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

A nucleic acid fragment encoding a “biologically-active fragment of protease” can be prepared by isolating a fragment SEQ ID NOs: 10, 11, 12, 13, 14, 15, 16, 17 or 18 that encodes a protease having a biological activity of the proteases of the invention (the biological activities of the proteases of the invention are described above), expressing the encoded portion of protease (for example, by recombinant expression in vitro) and assessing the activity of the encoded fragment of protease.

The invention further encompasses nucleic acid molecules that differ from the nucleic acid sequences shown in SEQ ID NOs: 10, 11, 12, 13, 14, 15, 16, 17 or 18 due to degeneracy of the genetic code and thus encode the same proteases that are encoded by the nucleic acid sequences shown in SEQ ID NOs: 10, 11, 12, 13, 14, 15, 16, 17 or 18.

In addition to the fungal protease nucleic acid sequences shown in SEQ ID NOs: 10, 11, 12, 13, 14, 15, 16, 17 or 18, it will be appreciated by those skilled in the art that DNA sequence polymorphisms that lead to changes in the amino acid sequences of the protease polypeptides may exist within a population of various species. Such genetic polymorphisms in the protease genes may exist among individual fungal species within a population due to natural allelic variation. As used herein, the terms “gene” and “recombinant gene” refer to nucleic acid molecules comprising an open reading frame (ORF) encoding a protease, preferably a fungal protease. Such natural allelic variations can typically result in 1-5% variance in the nucleic acid sequence of the protease genes. Any and all such nucleic acid variations and resulting amino acid polymorphisms in the protease polypeptides, which are the result of natural allelic variation and that do not alter the biological activity of the protease polypeptides, are intended to be within the scope of the invention.

Moreover, nucleic acid molecules encoding proteases of the invention from other species, and, thus, that have a nucleic acid sequence that differs from the sequence SEQ ID NOs: 10, 11, 12, 13, 14, 15, 16, 17 or 18 are intended to be within the scope of the invention. Nucleic acid molecules corresponding to natural allelic variants and homologues of the protease cDNAs of the invention can be isolated based on their homology to the fungal protease nucleic acids disclosed herein using the fungal cDNAs, or a portion thereof, as a hybridization probe according to standard hybridization techniques under stringent hybridization conditions.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein (an enzyme) encoded by an allelic variant of a gene.

Accordingly, in another embodiment, an isolated nucleic acid molecule of the invention is at least 6 nucleotides in length and hybridizes under stringent conditions to the nucleic acid molecule comprising the nucleic acid sequence of SEQ ID NOs: 10, 11, 12, 13, 14, 15, 16, 17 or 18.

In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region.

As used herein, the term “hybridizes under stringent conditions” is intended to describe conditions for hybridization and washing under which nucleotide sequences at least 60% homologous to each other typically remain hybridized to each other. Homologs or other related sequences (e.g., orthologs, paralogs) can be obtained by low, moderate or high stringency hybridization with all or a portion of the particular fungal sequence as a probe using methods well known in the art for nucleic acid hybridization and cloning. Stringent conditions are known to those skilled in the art and can be found in Ausubel et al., (eds.), 1993, CURRENT PROTOCOLS IN MOLECULAR BIOLOGY, John Wiley & Sons, N.Y. (1989), 6.3.1-6.3.6 and Kriegler, 1990; GENE TRANSFER AND EXPRESSION, A LABORATORY MANUAL, Stockton Press, NY and Shilo & Weinberg, Proc Natl Acad Sci USA 78:6789-6792 (1981).

For example, nucleotide substitutions leading to amino acid substitutions at “non-essential” amino acid residues can be made in the sequence of SEQ ID NOs: 10, 11, 12, 13, 14, 15, 16, 17 or 18. A “non-essential” amino acid residue is a residue that can be altered from the wild-type sequences of the proteases of the invention without altering their biological activity, whereas an “essential” amino acid residue is required for such biological activity.

As used herein, the term “biological activity” or “functional activity” refers to the natural or normal function of the proteases of the invention, for example the ability to degrade other proteins. Amino acid residues that are conserved among the proteases of the invention are predicted to be particularly non-amenable to alteration. Amino acids for which conservative substitutions can be made are well known within the art. The person skilled in the art will recognize that each codon in a nucleic acid (except AUG, which is ordinarily the only codon for methionine) can be modified to yield a functionally identical molecule by standard techniques. Furthermore, individual substitutions, deletions or additions which alter, add or delete a single amino acid or a small percentage of amino acids (typically less than 5%, more typically less than 1%) in an encoded sequence are “conservative mutations” where the alterations result in the substitution of an amino acid with a chemically similar amino acid.

Another aspect of the invention pertains to nucleic acid molecules encoding the proteases of the invention that contain changes in amino acid residues that are not essential for activity. Such proteases of the invention differ in amino acid sequence from SEQ ID NOs: 1, 2, 3, 4, 5, 6, 7, 8 or 9 yet retain biological activity. In one embodiment, the isolated nucleic acid molecule comprises a nucleotide sequence encoding a protease, wherein the protease comprises an amino acid sequence at least about 45% homologous to the amino acid sequences of SEQ ID NOs: 1, 2, 3, 4, 5, 6, 7, 8 or 9. Preferably, the protease encoded by the nucleic acid molecule is at least about 60% homologous to SEQ ID NOs: 1, 2, 3, 4, 5, 6, 7, 8 or 9; more preferably at least about 70% homologous to SEQ ID NOs: 1, 2, 3, 4, 5, 6, 7, 8 or 9; still more preferably at least about 80% homologous to SEQ ID NOS: 1, 2, 3, 4, 5, 6, 7, 8 or 9; even more preferably at least about 90% homologous to SEQ ID NOs: 1, 2, 3, 4, 5, 6, 7, 8 or 9; and most preferably at least about 95% homologous to SEQ ID NOs: 1, 2, 3, 4, 5, 6, 7, 8 or 9.

An isolated nucleic acid molecule encoding a protease of the invention homologous to the protein of SEQ ID NOs: 1, 2, 3, 4, 5, 6, 7, 8 or 9 can be created by introducing one or more nucleotide substitutions, additions or deletions into the nucleic acid sequence of SEQ ID NOs: 10, 11, 12, 13, 14, 15, 16, 17 or 18 such that one or more amino acid substitutions, additions or deletions are introduced into the encoded protease.

Mutations can be introduced into SEQ ID NOs: 10, 11, 12, 13, 14, 15, 16, 17 or 18 by standard techniques, such as site-directed mutagenesis, PCR-mediated mutagenesis and DNA shuffling. Preferably, conservative amino acid substitutions are made at one or more predicted, non-essential amino acid residues. A “conservative amino acid substitution” is a new amino acid that has similar properties and is one in which the amino acid residue is replaced with an amino acid residue having a similar side chain. Non-conservative substitutions refer to a new amino acid, which has different properties. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, hydroxyproline, phenylalanine, methionine, tryptophan), beta-branched side chains (e.g., threonine, valine, isoleucine) and aromatic side chains (e.g., tyrosine, phenylalanine, tryptophan, histidine). Thus, for a conservative substitution, a predicted non-essential amino acid residue in the protease of the invention is replaced with another amino acid residue from the same side chain family.

Alternatively, in another embodiment, mutations can be introduced randomly along all or part of a coding sequence of the protease of the invention, such as by saturation mutagenesis, and the resultant mutants can be screened for biological activity of the protease of the invention to identify mutants that retain activity. Following mutagenesis of SEQ ID NOs: 10, 11, 12, 13, 14, 15, 16, 17 or 18, the encoded protease can be expressed by any recombinant technology known in the art and the activity of the protein can be determined.

The host cell may be any of the host cells familiar to the person skilled in the art, including prokaryotic cells, eukaryotic cells, mammalian cells, insect cells, fungal cells, yeast cells and/or plant cells. As representative examples of appropriate hosts, there may be mentioned: bacterial cells, such as E. coli, Streptomyces, Bacillus subtilis, Bacillus cereus, Salmonella typhimurium and various species within the genera Pseudomonas, Streptomyces and Staphylococcus, fungal cells, such as Aspergillus, yeast such as any species of Pichia, Saccharomyces, Schizosaccharomyces, Schwanniomyces, including Pichia pastoris, Saccharomyces cerevisiae, or Schizosaccharomyces pombe, insect cells such as Drosophila S2 and Spodoptera 5/9, animal cells such as CHO, COS or Bowes melanoma and adenoviruses. Preferred host cells include Pichia pastoris, Aspergillus oryzae, Saccharomyces cerevisiae, and/or Kluveromyces lactis. The selection of an appropriate host is within the abilities of the person skilled in the art.

For example in order to promote production of proteolytic activity at neutral pH, A. oryzae will be grown in liquid media containing protein as the sole nitrogen source [collagen (animal) or soy meal (vegetal)]. To promote production of proteolytic activity at acidic pH, A. oryzae will be grown in liquid media containing a protein source dissolved in 68 mM citrate buffer (pH 3.5). After fungal growth, culture supernatants will be collected and dried by freeze-drying (lyophilisation). Aspergillus oryzae strain that over expresses the genes coding for enzymes of interest of the present invention, such as AfuS28, SedB, SedC, SedA, SedD and other proteases having the activity at neutral or acidic pH) will be engineered with the ultimate goal to design an optimal combination of enzymes for treatment based on fungal extracts. For instance, A. oryzae strains over producing DppIV was engineered by ectopic integration of the DPPIY gene in the genome of the fungus (Doumas A., van den Broek P., Affolter M., Monod M., 1998. Characterization of the prolyl dipeptidyl peptidase gene (dppIV) from the koji mold Aspergillus oryzae. Applied and Environmental Microbiology 64 (12), pp. 4809-15). It would also be possible to mix extracts from neutral and acidic pH cultures.

The production of a functional protein is intimately related to the cellular machinery of the organism producing the protein. The eukaryotic yeast, the methanoltrophic Pichia pastoris is typically used as the “factory” of choice for the expression of many proteins. P. pastoris has been developed to be an outstanding host for the production of foreign proteins since its alcohol oxidase promoter was isolated and cloned: The P. pastoris transformation was first reported in 1985. The P. pastoris heterologous protein expression system was developed by Phillips Petroleum, see, e.g., U.S. Pat. Nos. 4,855,231, 4,857,467, 4,879,231 and 4,929,555, each of which is incorporated herein by reference. Compared to other eukaryotic expression systems, Pichia offers many advantages, because it does not have the endotoxin problem associated with bacteria or the viral contamination problem of proteins produced in animal cell cultures. Furthermore, P. pastoris can utilize methanol as a carbon source in the absence of glucose. The P. pastoris expression system uses the methanol-induced alcohol oxidase (AOX1) promoter, which controls the gene that codes for the expression of alcohol oxidase, the enzyme that catalyzes the first step in the metabolism of methanol. This promoter has been characterized and incorporated into a series of P. pastoris expression vectors. Since the proteins produced in P. pastoris are typically folded correctly and secreted into the medium, the fermentation of genetically engineered P. pastoris provides an excellent alternative to E. coli expression systems. Furthermore, P. pastoris has the ability to spontaneously glycosylate expressed proteins, which also is an advantage over E. coli.

In one aspect, the nucleic acid sequences or vectors of the invention are introduced into the host cells, thus, the nucleic acids enter the host cells in a manner suitable for subsequent expression of the nucleic acid. The method of introduction is largely dictated by the targeted cell type.

Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.).

Another aspect of the invention pertains to vectors, preferably expression vectors, containing a nucleic acid encoding a protease of the invention, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of used in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The invention also encompasses a transformed host cell comprising nucleic acid sequences encoding the proteases of the invention, e.g., SEQ ID NOs: 10, 11, 12, 13, 14, 15, 16, 17 or 18.

Where appropriate, the engineered host cells can be cultured in conventional nutrient media modified as appropriate for activating promoters, selecting transformants or amplifying the nucleic acids coding for the proteases of the invention. The culture conditions, such as temperature, pH and the like, are those previously used with the host cell selected for expression and will be apparent to the person skilled in the art. The clones which are identified as having the specified enzyme activity may then be sequenced to identify the polynucleotide sequence encoding an enzyme having the enhanced activity. Following transformation of a suitable host cell and growth of the host cell to an appropriate cell density, the selected promoter may be induced by appropriate means (e.g., temperature shift or chemical induction) and the cells may be cultured for an additional period to allow them to produce the desired enzyme composition.

Host cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to the person skilled in the art. The expressed enzyme composition can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

The invention provides also a method for overexpressing recombinant proteases of the invention in a host cell comprising expressing a vector comprising a nucleic acid of the invention, e.g., an exemplary nucleic acid of the invention, including, e.g., SEQ ID NO: 10, 11, 12, 13, 14, 15, 16, 17 or 18 and biologically active fragments thereof, naturally occurring allelic variants thereof, or sequences having at least 70% of identity. The overexpression can be effected by any means, e.g., use of a high activity promoter, a dicistronic vector or by gene amplification of the vector.

The nucleic acid molecules of the invention can be expressed, or overexpressed, in any in vitro or in vivo expression system. Any cell culture systems can be employed to express, or over-express, recombinant protease, including bacterial, insect, yeast, fungal or mammalian cultures. Over-expression can be effected by appropriate choice of promoters, enhancers, vectors (e.g., use of replicon vectors, dicistronic vectors (see, e.g., Gurtu (1996) Biochem. Biophys. Res. Commun. 229:295-8), media, culture systems and the like. In one aspect, gene amplification using selection markers, e.g., glutamine synthetase (see, e.g., Sanders (1987) Dev. Biol. Stand. 66:55-63), in cell systems are used to overexpress the protease of the invention. Additional details regarding this approach are in the public literature and/or are known to the person skilled in the art, e.g., EP 0659215 (WO 9403612 A1) (Nevalainen et al); Lapidot (1996) J. Biotechnol. Nov. 51:259-64; Lüthi (1990) Appl. Environ. Microbiol. September 56:2677-83 (1990); Sung (1993) Protein Expr. Purif. June 4:200-6 (1993).

Alternatively, if it is desired to produce the proteases with other microorganisms than Aspergillus fumigatus, it is possible that the genetic information of Aspergillus fumigatus, which has been found initially by extensive screening and which has been proven to be a suitable source of the proteases of the invention, can be transferred to another microorganism which is normally used for the production of proteases, such as Pichia pastoris or Aspergillus oryzae that overexpresses the proteases of the invention, thereby providing the desired enzyme composition.

Further alternative to recombinant expression, a protease of the invention can be synthesized chemically using standard peptide synthesis techniques and purified using standard peptide purification techniques known to the person skilled in the art. In other aspects, fragments or portions of the polypeptides may be employed for producing the corresponding full-length polypeptide by peptide synthesis; therefore, the fragments may be employed as intermediates for producing the full-length polypeptides.

A “purified” polypeptide or protein or biologically-active fragment thereof is substantially free from chemical precursors or other chemicals when chemically synthesized. The language “substantially free of chemical precursors or other chemicals” includes preparations of the proteases of the invention in which the protease is separated from chemical precursors or other chemicals that are involved in the synthesis of the protease. For example, the proteases of the invention have less than about 30% (by dry weight) of chemical precursors or non-protease chemicals, more preferably less than about 20%, still more preferably less than about 10%, and most preferably less than about 5% chemical precursors or non-protease chemicals. Furthermore, “substantially free of chemical precursors or other chemicals” would include oxidation byproducts. The person skilled in the art would know how to prevent oxidation, for example, by keeping chemicals in an oxygen free environment.

In another embodiment, the enzyme composition of the invention can be derived from Aspergillus species, Penicillium species, Fusarium species, Saccharomyces species, and/or Kluveromyces species. Preferably the enzyme composition of the invention is derived from Aspergillus fumigatus, Aspergillus oryzae, Aspergillus niger, Aspergillus clavatus, Aspergillus glaucus, Aspergillus ornatus, Aspergillus cervinus, Aspergillus restrictus, Aspergillus ochraceus, Aspergillus candidus, Aspergillus flavus; Aspergillus wentii, Aspergillus cremeus, Aspergillus sparsus, Aspergillus versicolor, Aspergillus nidulans, Aspergillus ustus, Aspergillus flavipes, Aspergillus terreus, Penicillium roqueforti, Penicillium candidum, Penicillium notatum, Penicillium camemberti, Penicillium glaucus, Penicillium expansum, Penicillium digitatum, Penicillium chrysogenum, Penicillium citrinum, Penicillium commune, Penicillium decumbens, griseofulvum, Penicillium purpurogenum, Penicillium rugulosum, Penicillium verrucolosum, Fusarium venenatum, Saccharomyces cerevisiae, and/or Kluveromyces lactis.

As used herein the term “derived” encompasses the terms “originated from”, “obtained” or “obtainable from”, and “isolated from” and as used herein means that the polypeptide, for example a protease, encoded by a nucleic acid is produced from a cell in which the nucleic acid is naturally present or in which the nucleic acid has been inserted.

The proteases of the enzyme composition of the invention can be isolated from cells, such as Aspergillus species, Penicillium species, Fusarium species, Saccharomyces species, and/or Kluveromyces species or culture supernatants by an appropriate purification scheme using appropriate protein purification techniques known to the person skilled in the art.

An “isolated” or “purified” polypeptide or protein or biologically-active fragment thereof is substantially free of cellular material or other contaminating proteins from the cell from which the protease of the invention is derived.

The language “substantially free of cellular material” includes preparations of proteases of the invention in which the protease is separated from cellular material of the cells from which it is isolated or recombinantly-produced. For example the proteases of the invention have less than about 30% (by dry weight) of cellular material (or a contaminating protein), more preferably less than about 20%, still more preferably less than about 10%, and most preferably less than about 5% of cellular material (or a contaminating protein). When the protease of the invention or biologically-active fragment thereof is recombinantly-produced, it is also preferably substantially free of any constituent of the culture medium, e.g., culture medium components may represent less than about 20%, more preferably less than about 10%, and most preferably less than about 5% of the protease preparation.

Usually, the industrial production of enzymes is performed in a technical fermentation way using suitable microorganisms (bacteria, moulds, fungi). Usually the strains are recovered from natural ecosystems according to a special screening protocol, isolated as pure cultures as well as improved in their properties with respect to the enzyme spectrum and biosynthesis performance (volume/time yield). Enzyme production may also be carried out by methods developed in the future.

In a further embodiment, the present invention also encompasses a fungal enzyme extract, which comprises the enzyme composition according to the invention. Thus the fungal enzyme extract, comprising the enzyme composition according to the invention, can have the same or similar uses as disclosed herein for the enzyme composition of the invention. The fungal enzyme extract of the invention is derived from Aspergillus species, Penicillium species, Fusarium species, Saccharomyces species, and/or Kluveromyces species, and preferably from Aspergillus fumigatus, Aspergillus oryzae, Aspergillus niger, Aspergillus clavatus, Aspergillus glaucus, Aspergillus ornatus, Aspergillus cervinus, Aspergillus restrictus, Aspergillus ochraceus, Aspergillus candidus, Aspergillus flavus; Aspergillus wentii, Aspergillus cremeus, Aspergillus sparsus, Aspergillus versicolor, Aspergillus nidulans, Aspergillus ustus, Aspergillus flavipes, Aspergillus terreus, Penicillium roqueforti, Penicillium candidum, Penicillium notatum, Penicillium camemberti, Penicillium glaucus, Penicillium expansum, Penicillium digitatum, Penicillium chrysogenum, Penicillium citrinum, Penicillium commune, Penicillium decumbens, griseofulvum, Penicillium purpurogenum, Penicillium rugulosum, Penicillium verrucolosum, Fusarium venenatum, Saccharomyces cerevisiae, and/or Kluveromyces lactis.

Encapsulation of the fungal extract is an option to circumvent the problem of possible sensitivity of enzymes to stomach environment.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

The foregoing description will be more fully understood with reference to the following Examples. Such Examples, are, however, exemplary of methods of practising the present invention and are not intended to limit the scope of the invention.

### EXAMPLES

### Strains and Plasmids.

Aspergillus fumigatus D141 (NRRL 6585; U.S. Department of Agriculture, Peoria, Ill.) was used in this study. All plasmid subcloning experiments were performed in E. coli XL1 blue using plasmid pKJ113 (Borg von Zepelin et al., 1998).

Pichia pastoris GS115 (Invitrogen, Carlsbad, Calif.) was used to produce heterologous (recombinant) peptidases.

Aspergillus fumigatus Growth Media.

Aspergillus fumigatus was routinely grown on malt agar or, to promote production of proteolytic activity at neutral pH, in liquid media containing protein as the sole nitrogen source (0.2% collagen) (Monod et al., 1991). The pH was approximately 7.0 and slightly increased to 7.5 during growth of the fungus. To promote production of proteolytic activity at acidic pH in collagen medium, 0.2% collagen was dissolved in 68 mM citrate buffer (pH 3.5). One liter flasks containing 200 ml of medium were inoculated with approximately 10⁸ spores and incubated for 70 h at 37° C. on an orbital shaker at 200 rpm.

### Recombinant Protease Production.

Recombinant A. fumigatus SedB was previously produced and purified from P. pastoris used as an expression system (Reichard et al., 2006). To construct P. pastoris strains producing AfuS28 (MER064064), amplified cDNA segments encoding N-terminal and C-terminal parts of the protein were obtained by PCR with a standard protocol (Jousson et al., 2004a; 2004b) using homologous sense and antisense primers (P1 and P2, P3 and P4, respectively, Table 1) and 200 ng of DNA prepared from 10⁶ clones of a cDNA library as a template (Reichard et al., 2006). P5 was used instead of P4 as antisense primer to obtain His-Tagged AfuS28. The PCR products were digested with XhoI/SacI and SacI/BglII, respectively, and inserted end to end into pKJ113 digested with XhoI/BamHI to generate the expression plasmids pAfuS28 and pAfuS28H-6. Pichia pastoris GS115 transformation with EcoRI linearized plasmidic DNA and transformants were selected as previously described (Borg von Zepelin, 2008).

For enzyme production, P. pastoris transformants were grown to near saturation (OD₆₀₀=10) at 30° C. in 10 ml of glycerol-based yeast media [0.1 M potassium phosphate buffer at pH 6.0, containing 10 g/L yeast extract, 20 g/L peptone, 13 g/L yeast nitrogen base without amino acids (Becton-Dickinson, Sparks, Md.), 10 ml/L glycerol and 40 mg/L biotin]. Cells were harvested and resuspended in 2 ml (200 ml) of the same medium with 5 ml/L methanol instead of glycerol and incubated for 2 days. Then, the culture supernatant was harvested after centrifugation (3000×g, 4° C., 5 min).

Salts and small molecular weight solutes were removed from 2.5 ml of P. pastoris culture supernatant by passing through a PD10 column (Amersham Pharmacia, Dübendorf, Switzerland) with 20 mM citrate buffer (pH 6.0) before testing for proteolytic activity. The supernatant of P. pastoris GS115 grown under the same conditions was used as a negative control for comparison.

TABLE 1
Primers for AfuS28 and AfuS28 antigen construct

| | | |
|---|---|---|
| P1: | 5′-GTTTCTCGAGCACTCATGCCCAGGGCGCCT | (SEQ ID NO: 19) |
| | T-3′ | |
| P2: | 5′-TGAGAGCTCCCAACCCGAACATCTC-3′ | (SEQ ID NO: 20) |
| P3: | 5′-TGGGAGCTCTCAAGCATTTTGACT-3′ | (SEQ ID NO: 21) |
| P4: | 5′-GTTTAGATCTCATGGCTTCCTATATTTGG | (SEQ ID NO: 22) |
| | G-3′ | |
| P5: | 5′-GTTTAGATCTCAGTGATGGTGATGGTGAT | (SEQ ID NO: 23) |
| | GTGGCTTCCTATATTTGGG-3′ | |
| P6: | 5′-GTTCCATGGGTGGCTTTGGCAGGATATGA | (SEQ ID NO: 24) |
| | AT-3′ | |
| P7: | 5′-CTTGGATCCTCATGGCTTCCTATATTTGG | (SEQ ID NO: 25) |
| | G-3′ | |

### Purification of Heterologously Produced AfuS28.

The secreted proteins from 250 ml of P. pastoris culture supernatant were concentrated by ultrafiltration to 6 ml using a Centricon Plus-70 (30 kDa cut-off) (Millipore, Volketswil, Switzerland). The 6×His tagged target protein was extracted with a Ni-NTA resin (Qiagen, Hilden, Germany) column with histidine elution buffer (50 mM histidine in PBS 1×) as previously described (Sarfati et al., 2006). Active fractions were pooled and concentrated by ultrafiltration using Amicon Ultra (Milipore 30000 kDa cut-off). Protein concentrations were measured by the method of Bradford with a commercial reagent (Bio-Rad).

In parallel, AfuS28 without His₆ tag was purified at 4° C. as following: secreted proteins from 250 ml of P. pastoris culture supernatant were concentrated by ultrafiltration to 6 ml using a Centricon Plus-70 (30 kDa cut-off) (Millipore, Volketswil, Switzerland). Thereafter, the concentrate was desalted with PD10 column (Amersham) and applied to a DEAE-Sepharose column which was previously equilibrated with a 100 mM Na acetate buffer (pH 5.8). After washing the column with the same buffer, the recombinant protein was eluted with a 100 mM sodium acetate buffer (pH 3.8). Enzymatic activity was tested with Ala-Ala-Pro-p-nitroanilide (Ala-Ala-Pro-pNA) as a substrate and active fractions were pooled.

### Quality Control of Purified AfuS28 (on Silver Stained SDS-PAGE Gels).

To assess the degree of purity of AfuS28, eluted fractions were pooled and 5 μl aliquots were migrated through a SDS-PAGE and stained with silver nitrate according to Chevallet M. protocol (Chevallet et al., 2006).

### Antigen Preparation for Immunization of Rabbits.

A 253 amino acid large peptide corresponding to the C-terminal part of AfuS28 was produced using plasmid pET-11aH6, a derivative of pET-11 a made for His6-tagged large peptide production (Reichard et al., 2006). Sense and antisense primers P6 and P7 (Table 1) were used to amplify DNA from plasmid pAfuS28 encoding heterologous AfuS28. The PCR products were digested with NcoI and BamHI and cloned into the NcoI and BamHI sites of pET-11aH6. The resulting plasmid was termed pAGAfuS28.

The corresponding heterologous 6×His tagged peptide was produced in E. coli BL21 transformed with pAgAfuS28. Cells were grown at 37° C. to an OD₆₀₀ of 0.6 and 6×His tagged peptide expression was induced by adding IPTG to a 0.1 mM final concentration after which incubation was continued for an additional 4 h at 37° C. Cells were collected by centrifugation (4,500×g, 4° C., 15 min), and the 6×His tagged peptides were extracted by lysis with guanidine hydrochloride buffer and Ni-NTA resin affinity (Qiagen, Hilden, Germany) columns according to the manufacturer. The column was washed with 0.1 M sodium phosphate buffer (pH 5.9) containing 8 M urea. Thereafter, antigen was eluted with the same buffer adjusted at pH 4.5. Rabbit antisera were made by Eurogentec (Liège, Belgium) by using the purified AfuS28 polypeptide chain as an antigen.

### Western Blot Analysis of Native and Recombinant AfuS28.

AfuS28 samples with or without prior N-glycosidase F digestion (Doumas et al., 1998), were analyzed by Western blotting of SDS-PAGE gels (12.5%). Western blots were immunodeveloped using anti AfuS28 antiserum raised in rabbits, and alkaline phosphatase-conjugated goat anti-rabbit IgG (Bio-Rad, Hercules, Calif.).

### Proteolytic Activities.

Endoproteolytic activities were measured with 50 μA A. fumigatus and P. pastoris culture supernatants and 50 μl of 0.2% resorufin-labeled casein at different pHs in sodium citrate buffer (50 mM final concentration; pH 2.0 to 7.0) in a total volume of 0.5 ml. After incubation at 37° C., the undigested substrate of the enzyme-substrate mix was precipitated by trichloroacetic acid (4% final concentration) and separated from the supernatant by centrifugation. Subsequently, 500 μl of Tris-HCl buffer (500 mM; pH 9.4) were added to the collected supernatant (neutralization step) and the A₅₇₄ of the mixture (1 ml) was measured. A blank was performed with 50 μl P. pastoris GS115 culture supernatant. For practical purposes, one milliunit of endoproteolytic activity was arbitrarily defined as producing an increase in absorbance of 0.001 per min in a proteolytic assay (1 ml) at optimal pH for activity. The assays were performed in triplicates. Exoproteolytic activites were tested with synthetic substrates supplied by Genecust (Dunedange, Luxembourg). Stock solutions were prepared at 100 mM concentration and stored at −20° C. AP-pNA (Ala-Pro-p-nitroanilide), AA-pNA, FPA-pNA, AAP-pNA and AAAP-pNA were dissolved in water/DMSO. The reaction mixture contained a concentration of 10 mM substrate and the enzyme preparation (between 0.1 to 1.0 μg per assay) in 50 μl of 100 mM acetate buffer at different pH values. After incubation at 37° C. for 10 min, the reaction was terminated by adding 5 μl of glacial acetic acid and then 0.9 ml of water. The released pNA was measured by spectrophotometry at λ=405 nm. A control with substrate but without enzyme was carried out in parallel. The AfuS28 activities were expressed in mU (μmoles of released pNA/min) using Ala-Ala-Ala-Pro-pNA as a substrate.

The ability of AfuS28 to digest proline-rich peptides was investigated on three substrates:

NPY 1-36 (neuropeptideY, YPSKPDNPGEDAPAEDMARYYSALRHYINLITRQRY-NH2,) (SEQ ID NO:26), NPY 3-36
(SKPDNPGEDAPAEDMARYYSALRHYINLITRQRY-NH2) (SEQ ID NO:27) (Bachem) and bradykinin (RPPGFSPFR) (SEQ ID NO:28) (Sigma, Bradykinin acetate B3259-5MG). NPY1-36 and NPY3-36 were dissolved in deionized water at 1.2 nmol/μl concentration and bradykinin was dissolved at 94 nmol/μ1 (100 μg/μl). To measure by mass spectrometry the degradation of both NPY1-36 and NPY3-36, a solution containing 5 nmol of substrate with 1.8 mU of AfuS28 in an acidic buffer (0.015% formic acid, pH 4) was prepared. Degradation of bradykinin by AfuS28 was performed with 4 nmol/μl substrate as a final concentration and a control without AfuS28 was carried out in parallel. The enzymatic activity at 37° C. was decreased at different times by adding 0.5% formic acid and stopped by freezing with liquid nitrogen. The solutions were diluted 5 to 10 fold in H₂O:acetonitrile 50:50 with 0.1% formic acid and analyzed by mass spectrometry. They were infused in a LTQ-Orbitrap instrument (ThermoFisher, Bremen, Germany) via a TriVersa Nanomate (Advion Biosciences, Norwich, UK) system. Mass spectra were acquired in MS survey mode at a resolution of 60′000 (at 400 m/z) and accuracy better than 5 ppm.
Precipitation and Separation of Proteins from A. fumigatus Culture Supernatants by 1D-SDS-PAGE.

The mycelium was separated from culture medium by paper filtration (Miracloth from Calbiochem). Thereafter, 50 ml of supernatant were centrifuged for 10 minutes at 5000×g to remove debris, followed by a concentration step to 1 ml using a Centricon Plus-70 with a 10′000 Da cut-off. Concentrated media were precipitated as follows: 0.9 ml of 0.2% (w/v) sodium deoxycholate was mixed with 100 μl of concentrated medium and incubated for 10 min at room temperature. 100 μl of 6.1 N TCA was added to this mixture and was gently shaken. The sample was incubated for 10 min at 4° C., and then centrifuged at 13000 rpm for 10 min to obtain a pellet. After removal of the supernatant, the pellet was washed twice with 100% acetone and dried.

For 1D-SDS-PAGE analysis, the pellet was dissolved in 20 μl of 20 mM Tris-HCl, pH 7.4 and mixed with SDS sample buffer. Proteins were separated on a 12% SDS polyacrylamide gel followed by staining with Coomassie brilliant blue R-250 (Bio-Rad). The total optical density in every lane was determined by densitometry and used to calibrate sample loadings onto a preparative gel. For protein digestion (shotgun experiments), equal amounts of protein for every sample were subjected to limited electrophoretic separation on a 10% minigel, i.e. the migration was stopped after the front had moved by about 2.5 cm into the separating gel. At this time all bands up to 250 kDa of a prestained molecular weight marker had moved into the gel and were distinguishable. Gels were fixed for 10 min, partially stained with Coomassie Brilliant blue G (15 min) and then destained for 30 min. Every lane was cut into 4-5 sections beginning with high molecular weights.

### Digestion and MS Analysis: Shotgun MS Experiments

The mycelium was separated from culture medium by paper filtration (Miracloth from Calbiochem). Thereafter, 50 ml of supernatant were centrifuged for 10 minutes at 5000×g to remove debris, followed by a concentration step to 1 ml using a Centricon Plus-70 with a 10′000 Da cut-off. Concentrated media were precipitated as follows: 0.9 ml of 0.2% (w/v) sodium deoxycholate was mixed with 100 μl of concentrated medium and incubated for 10 min at room temperature. 100 μl of 6.1 N TCA was added to this mixture and was gently shaken. The sample was incubated for 10 min at 4° C., and then centrifuged at 13000 rpm for 10 min to obtain a pellet. After removal of the supernatant, the pellet was washed twice with 100% acetone and dried.

For 1D-SDS-PAGE analysis, the pellet was dissolved in 20 μl of 20 mM Tris-HCl, pH 7.4 and mixed with SDS sample buffer. Proteins were separated on a 12% SDS polyacrylamide gel followed by staining with Coomassie brilliant blue R-250 (Bio-Rad). The total optical density in every lane was determined by densitometry and used to calibrate sample loadings onto a preparative gel. For protein digestion (shotgun experiments), equal amounts of protein for every sample were subjected to limited electrophoretic separation on a 10% minigel, i.e. the migration was stopped after the front had moved by about 2.5 cm into the separating gel. At this time all bands up to 250 kDa of a prestained molecular weight marker had moved into the gel and were distinguishable. Gels were fixed for 10 min, partially stained with Coomassie Brilliant blue G (15 min) and then destained for 30 min. Every lane was cut into 4-5 sections beginning with high molecular weights.

Database Searching with MS Data

From raw files, MS/MS spectra were de-isotoped and exported as mgf files (Mascot Generic File, text format) using MascotDistiller 2.1.1 (Matrix Science, London, UK). MS/MS spectra were searched with Mascot (Matrix Science, London, UK; version 2.2.0) against the UNIPROT database (www.expasy.org) selected for Fungi assuming the digestion enzyme trypsin and one missed cleavage. The database release used was of Apr., 23th 2008 (5,939,836 sequences, Fungi: 358052 sequences). Mascot was searched with a fragment ion mass tolerance of 0.50 Da and a parent ion tolerance of 10.0 PPM. Iodoacetamide derivative of cysteine was specified in Mascot as a fixed modification. N-terminal acetylation of protein, deamidation of asparagine and glutamine, and oxidation of methionine were specified in Mascot as variable modifications.

### Criteria for Protein Identification

Scaffold (version Scaffold 2_{—}05_{—}01, Proteome Software Inc., Portland, Oreg.) was used to validate MS/MS based peptide and protein identifications. Peptide identifications were accepted if they could be established at greater than 90.0% probability as specified by the Peptide Prophet algorithm (Keller, A et al Anal. Chem. 2002; 74(20):5383-92). Protein identifications were accepted if they could be established at greater than 95.0% probability and contained at least 1 identified peptide. Protein probabilities were assigned by the Protein Prophet algorithm (Nesvizhskii, AI Anal Chem. 2003 Sep. 1; 75(17):4646-58). Proteins that contained similar peptides and could not be differentiated based on MS/MS analysis alone were grouped to satisfy the principles of parsimony.

### Comprehensive Identification of Proteins Secreted by A. Fumigatus at Different pH Values.

Aspergillus fumigatus grew well at 30° C. in a medium containing 0.2% collagen protein as a sole carbon and nitrogen source at both pH 7.0 and pH 3.5. After two days of growth, clarification of the culture medium was observed. At this time, the amount of protein was 20-50 μg·ml⁻¹ in culture supernatants at both pH values. Concomitantly, a substantial proteolytic activity was measured using resorufin-labelled casein as substrate. Substantial activities on APF-pNA, AAP-pNA and AAAP-pNA were also detected in culture supernatant. Activity on AP-pNA was detected in the culture supernatant at pH 7.0, but not at pH 3.5.

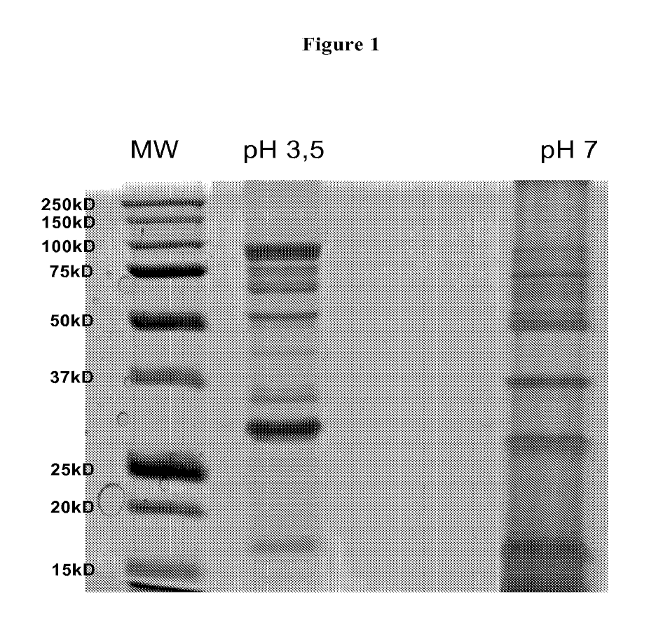

SDS-PAGEs of proteins precipitated from culture supernatants at pH 7.0 and pH 3.5 showed complex band patterns with major differences (FIG. 1). In an attempt to map a maximum number of proteins secreted by A. fumigatus at different pH values, a systematic shotgun protein identification experiment was undertaken. After sample fractionation by limited 1D SDS-PAGE electrophoresis, five identical gel bands corresponding to different molecular weights were excised from every lane and proteins were in-gel digested. After peptide gel extraction, every fraction was analysed by LC-MS/MS on a LTQ-orbitrap mass spectrometer.

Lists of spectra for each lane were merged in order to obtain a dataset that was used for searching a fungi sequence database (SPTrEMB1, Apr. 23, 2008, Taxonomy Fungi: 358052 sequences, supplementary table 2, 3 and 4). Total numbers of MS/MS spectra assigned to peptides after database search were 3292 and 2352 for A. fumigatus samples at pH 7.0 and pH 3.5, respectively. Spectral counting coupled with redundant sampling of a mixture has been established as a reliable method to quantify protein abundances in shotgun experiments (Liu, 2004). Here we have assumed that the number of matched spectra represents at least semi-quantitative estimates of the relative protein abundances between samples. The most abundant protein identified in the A. fumigatus sample at pH 7.0 was the dipeptidyl peptidase DppV (XP 755237) with 466 spectra which correspond to 38 unique peptide sequences. The most abundant protein identified in the A. fumigatus sample at pH 3.5 was a tripeptidyl peptidase called sedolisin B (SedB) with 315 spectra which correspond to 17 unique peptide sequences (table 2). Extensive details on the results of identifications can be found in Table 3. Overall, 171 different sequences were matched in the shotgun experiment.

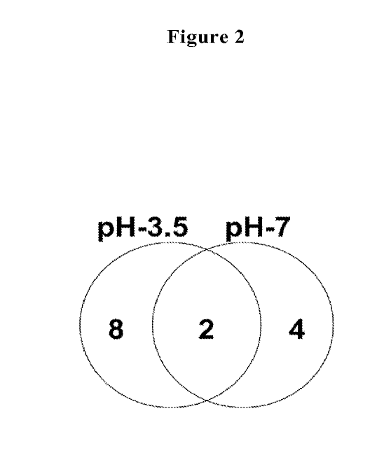

As expected, proteases constituted a significant fraction of all identified proteins, with (5 endo- and 10 exoproteases) (Table 2). Furthermore, proteases accounted for 30 to 40% of total matched spectra in the shotgun analysis, a fact that highlights their quantitative dominance. These proteases fall into two only slightly overlapping groups corresponding to the acidic and neutral pH secretomes (FIG. 2). Alkaline serine protease Alp1 (XP_{—}751651), DppV (XP_{—}755237) and leucine aminopeptidase Lap2 (XP_{—}748386) were three major proteases only secreted at pH 7.0. Aspartic endoprotease Pep1 (XP_{—}753324), SedB (XP_{—}746536) and serine carboxypeptidase Scp1 (XP_{—}753901) were three major proteases only secreted at pH 3.5. A putative glutamic endoprotease (XP 748619) ortholog of A. niger Aspergillopepsin II, SedD (XP751432) was also to be found in culture supernatant at pH 3.5, but in an amount lower than those of the three preceeding cited major acidic proteases. Only one putative serine protease of the S28 family, called here AfuS28 and homologous to a previously described A. niger prolylendopeptidase (XP_{—}001392567), was secreted in similar amounts at both pH values (Table 2). A total of 100 identified proteins were hydrolytic enzymes and other hydrolases detected were glycosidases (mannosidase, glutaminase, beta-1,3-endoglucanase) lipases and acid phosphatases. For nineteen sequences, no function could be assigned based on sequence similarity.

TABLE 2
Proteases secreted massively by A. fumigatus on media containing collagen at pH 3.5 and 7 during 70 h growth under shaking at
30° C. Numbers of matched spectra give a semiquantitative measure of protein amounts.

| | | As | | | Number of spectra detected | |
|---|---|---|---|---|---|---|
| | | named | | | by Mass spectrometry | |
| | | in the | Accession | Molecular | A. Fumigatus | A. Fumigatus |
| Family | Identified Proteins (enzymes massively secreted only) | Text | Number | Weight | pH-3.5 | pH-7 |
| S53 | Tripeptidyl-peptidase - Aspergillus fumigatus A1163 | SedB | B0YEL1 | 66 kDa | 315 | 0 |
| A1 | Aspartic endopeptidase Pep1 - Aspergillus fumigatus A1163 | Pep1 | B0Y1V8 | 42 kDa | 240 | 0 |
| S10 | Carboxypeptidase S1, putative - Aspergillus fumigatus A1163 | | B0Y1L0 | 54 kDa | 123 | 0 |
| S10 | Carboxypeptidase 5 - Aspergillus fumigatus (Sartorya | | Q5VJG7 | 60 kDa | 45 | 0 |
| | fumigata) | | | | | |
| S10 | Carboxypeptidase 4 - Aspergillus fumigatus (Sartorya | | Q5VJG8 | 58 kDa | 43 | 0 |
| | fumigata) | | | | | |
| S53 | Tripeptidyl peptidase A - Aspergillus fumigatus A1163 | SedD | B0Y502 | 64 kDa | 26 | 0 |
| S10 | Carboxypeptidase S1, putative - Aspergillus fumigatus A1163 | | B0YA52 | 61 kDa | 22 | 0 |
| G1 | Aspergillopepsin, putative - Aspergillus fumigatus A1163 | G1 | B0Y015 | 28 kDa | 23 | 0 |
| S28 | Serine peptidase, putative - Aspergillus fumigatus A1163 | AfuS28 | B0XT80 | 59 kDa | 57 | 45 |
| M36 | Elastinolytic metalloproteinase Mep - Aspergillus fumigatus | Mep | B0Y9E2 | 69 kDa | 0 | 7 |
| | A1163 | | | | | |
| S8A | Autophagic serine protease Alp2 - Aspergillus fischerianus | Alp2 | A1DER5 | 53 kDa | 0 | 10 |
| S9 | Extracellular dipeptidyl-peptidase Dpp4 - Aspergillus | DppIV | B0Y6C5 | 86 kDa | 0 | 54 |
| | fumigatus A1163 | | | | | |
| M28A | Aminopeptidase Y LAP2, putative - Aspergillus fumigatus | Lap2 | B0XX53 | 54 kDa | 0 | 133 |
| | A1163 | | | | | |
| S8A | Alkaline serine protease Alp1 - Aspergillus fumigatus A1163 | Alp1 | B0Y708 | 42 kDa | 0 | 433 |
| S9 | Secreted dipeptidyl peptidase Dpp5 - Aspergillus fumigatus | DppV | B0XRV0 | 80 kDa | 13 | 466 |
| | A1163 | | | | | |

TABLE 3
Comparison between secreted protein on pH 3.5 and 7 get by Shotgun proteomics analysis

| | | | Secretome A. fumigatus | |
|---|---|---|---|---|
| | Accession | Molecular | (number of spectra detected by MS) | |
| Identified Proteins (171) | Number | Weight | pH-3.5 | pH-7 |
| Secreted dipeptidyl peptidase - Aspergillus fumigatus A1163 | B0XRV0 | 80 kDa | 13 | 466 |
| Alkaline serine protease Alp1 - Aspergillus fumigatus A1163 | B0Y708 | 42 kDa | 0 | 433 |
| Tripeptidyl-peptidase - Aspergillus fumigatus A1163 | B0YEL1 | 66 kDa | 315 | 0 |
| Mannosidase MsdS - Aspergillus fumigatus A1163 | B0XMT4 | 54 kDa | 93 | 170 |
| Aspartic endopeptidase Pep1 - Aspergillus fumigatus A1163 | B0Y1V8 | 42 kDa | 240 | 0 |
| Beta-D-glucoside glucohydrolase - Aspergillus fumigatus A1163 | B0YB65 | 78 kDa | 61 | 131 |
| Glutaminase GtaA - Aspergillus fumigatus A1163 | B0XYT5 | 76 kDa | 20 | 161 |
| FG-GAP repeat protein, putative - Aspergillus fumigatus (Sartorya | Q4WK08 | 34 kDa | 4 | 171 |
| fumigata) | | | | |
| Aminopeptidase Y, putative - Aspergillus fumigatus A1163 | B0XX53 | 54 kDa | 0 | 133 |
| Mycelial catalase Cat1 - Aspergillus fumigatus A1163 | B0Y0G0 | 80 kDa | 5 | 129 |
| Carboxypeptidase S1, putative - Aspergillus fumigatus A1163 | B0Y1L0 | 54 kDa | 123 | 0 |
| FAD-dependent oxygenase, putative - Aspergillus fumigatus A1163 | B0XX33 | 55 kDa | 67 | 42 |
| Extracellular lipase, putative - Aspergillus fumigatus A1163 | B0Y214 | 31 kDa | 9 | 101 |
| Glucan 1,4-alpha-glucosidase, putative - Aspergillus fumigatus A1163 | B0XSV7 | 67 kDa | 83 | 35 |
| Serine peptidase, putative - Aspergillus fumigatus A1163 | B0XT80 | 59 kDa | 57 | 45 |
| Chitosanase precursor - Aspergillus fumigatus (Sartorya fumigata) | Q709P2 | 25 kDa | 104 | 0 |
| Beta-fructofuranosidase, putative - Aspergillus fumigatus A1163 | B0XT79 | 57 kDa | 50 | 32 |
| Major allergen Asp F1 - Aspergillus fumigatus A1163 | B0Y2B4 | 20 kDa | 70 | 0 |
| GPI-anchored cell wall beta-1,3-endoglucanase EglC - Aspergillus | B0XXF8 | 45 kDa | 33 | 44 |
| fumigatus A1163 | | | | |
| Carboxypeptidase 5 - Aspergillus fumigatus (Sartorya fumigata) | Q5VJG7 | 60 kDa | 45 | 0 |
| FAD/FMN-containing isoamyl alcohol oxidase MreA - Aspergillus | B0YD87 | 61 kDa | 36 | 21 |
| fumigatus A1163 | | | | |
| Isoamyl alcohol oxidase, putative - Aspergillus fumigatus (Sartorya | A4D9R5 | 61 kDa | 32 | 33 |
| fumigata) | | | | |
| Beta-N-acetylhexosaminidase NagA, putative - Aspergillus fumigatus | B0Y9W3 | 67 kDa | 37 | 24 |
| A1163 | | | | |
| Cell wall serine-threonine-rich galactomannoprotein Mp1 - | B0YEP2 | 27 kDa | 12 | 47 |
| Aspergillus fumigatus A1163 | | | | |
| Extracellular dipeptidyl-peptidase Dpp4 - Aspergillus fumigatus | B0Y6C5 | 86 kDa | 0 | 54 |
| A1163 | | | | |
| Beta glucosidase, putative - Aspergillus fumigatus A1163 | B0Y7Q8 | 95 kDa | 0 | 48 |
| Mannosidase I - Aspergillus fumigatus (Sartorya fumigata) | Q6PWQ1 | 55 kDa | 17 | 30 |
| Phytase, putative - Aspergillus fumigatus A1163 | B0YBU1 | 57 kDa | 48 | 0 |
| Cell wall protein, putative - Aspergillus fumigatus (Sartorya fumigata) | Q4WFT1 | 19 kDa | 0 | 46 |
| Exo-beta-1,3-glucanase, putative - Aspergillus fumigatus A1163 | B0XLY8 | 84 kDa | 6 | 36 |
| Beta-galactosidase, putative - Aspergillus fumigatus (Sartorya | Q4WS33 | 112 kDa | 38 | 0 |
| fumigata) | | | | |
| Carboxypeptidase 4 - Aspergillus fumigatus (Sartorya fumigata) | Q5VJG8 | 58 kDa | 43 | 0 |
| Putative uncharacterized protein - Aspergillus fumigatus A1163 | B0Y501 | 23 kDa | 37 | 7 |
| Acid phosphatase, putative - Aspergillus fumigatus A1163 | B0YEJ1 | 46 kDa | 44 | 0 |
| Thioredoxin reductase, putative - Aspergillus fumigatus A1163 | B0Y2V0 | 43 kDa | 12 | 25 |
| Putative uncharacterized protein - Aspergillus fumigatus A1163 | B0XVH5 | 42 kDa | 19 | 20 |
| 1,3-beta-glucanosyltransferase Bgt1 - Aspergillus fumigatus A1163 | B0XQR5 | 33 kDa | 31 | 0 |
| Oxidoreductase, FAD-binding, putative - Aspergillus fumigatus A1163 | B0XU27 | 50 kDa | 14 | 12 |
| Bifunctional catalase-peroxidase Cat2 - Aspergillus fumigatus A1163 | B0YAK0 | 84 kDa | 0 | 37 |
| Endonuclease/exonuclease/phosphatase family protein - Aspergillus | B0XY76 | 64 kDa | 3 | 29 |
| fumigatus A1163 | | | | |
| Beta galactosidase, putative - Aspergillus fumigatus A1163 | B0XNY2 | 112 kDa | 14 | 18 |
| Class V chitinase ChiB1 - Aspergillus fumigatus A1163 | B0YAM7 | 48 kDa | 0 | 30 |
| 1,3-beta-glucanosyltransferase Gel1 - Aspergillus fumigatus A1163 | B0XT72 | 48 kDa | 22 | 4 |
| Tripeptidyl peptidase A - Aspergillus fumigatus A1163 | B0Y502 | 64 kDa | 26 | 0 |
| IgE-binding protein - Aspergillus fumigatus A1163 | B0YDX1 | 20 kDa | 0 | 28 |
| Alpha-galactosidase, putative - Aspergillus fumigatus A1163 | B0YDJ1 | 56 kDa | 15 | 9 |
| Alpha-1,2-mannosidase family protein, putative - Aspergillus | B0XQJ8 | 93 kDa | 19 | 10 |
| fumigatus A1163 | | | | |
| Extracellular cell wall glucanase Crf1/allergen Asp F9 - Aspergillus | B0XNL0 | 40 kDa | 5 | 24 |
| fumigatus A1163 | | | | |
| Alpha-1,3-glucanase/mutanase, putative - Aspergillus fumigatus | B0XUS0 | 54 kDa | 0 | 22 |
| A1163 | | | | |
| Extracellular serine-rich protein, putative - Aspergillus fumigatus | B0XYK6 | 84 kDa | 0 | 23 |
| A1163 | | | | |
| Putative uncharacterized protein - Aspergillus fumigatus A1163 | B0YEP7 | 49 kDa | 0 | 27 |
| Extracellular arabinanase, putative - Aspergillus fumigatus A1163 | B0YDT3 | 45 kDa | 0 | 21 |
| 1,3-beta-glucanosyltransferase, putative - Aspergillus fumigatus | B0XVI5 | 59 kDa | 14 | 0 |
| A1163 | | | | |
| Alpha-glucosidase, putative - Aspergillus fumigatus A1163 | B0XNL6 | 99 kDa | 0 | 19 |
| Acid sphingomyelinase, putative - Aspergillus fumigatus A1163 | B0Y5K6 | 68 kDa | 0 | 23 |
| Cell wall protein PhiA - Aspergillus fumigatus (Sartorya fumigata) | A4FSH5 | 19 kDa | 4 | 19 |
| Carboxypeptidase S1, putative - Aspergillus fumigatus A1163 | B0YA52 | 61 kDa | 22 | 0 |
| Aspergillopepsin, putative - Aspergillus fumigatus A1163 | B0Y015 | 28 kDa | 23 | 0 |
| Alpha-L-arabinofuranosidase A - Aspergillus fumigatus A1163 | B0XUG6 | 72 kDa | 13 | 10 |
| Alpha,alpha-trehalose glucohydrolase TreA/Ath1 - Aspergillus | B0Y0F9 | 117 kDa | 14 | 4 |
| fumigatus A1163 | | | | |
| Alpha-galactosidase - Aspergillus fumigatus A1163 | B0Y224 | 47 kDa | 15 | 0 |
| Extracellular endo-polygalacturonase, putative - Aspergillus | Q4WBE1 | 38 kDa | 21 | 0 |
| fumigatus (Sartorya fumigata) | | | | |
| Endo-1,4-beta-xylanase - Aspergillus fumigatus A1163 | B0XXF3 | 24 kDa | 4 | 12 |
| Beta-1,6-glucanase, putative - Aspergillus fumigatus A1163 | B0Y9D8 | 51 kDa | 20 | 0 |
| Alpha-glucosidase AgdA, putative - Aspergillus fumigatus A1163 | B0Y6K4 | 108 kDa | 19 | 0 |
| Extracelular serine carboxypeptidase, putative - Aspergillus | B0XVM7 | 55 kDa | 7 | 6 |
| fumigatus A1163 | | | | |
| Endo-1,3(4)-beta-glucanase, putative - Aspergillus fumigatus A1163 | B0Y002 | 31 kDa | 0 | 15 |
| Endo-arabinase, putative - Aspergillus fumigatus A1163 | B0XU55 | 36 kDa | 0 | 16 |
| Putative uncharacterized protein - Aspergillus fumigatus A1163 | B0XP19 | 33 kDa | 9 | 4 |
| Allergen Asp f 15 precursor - Aspergillus fumigatus (Sartorya | AL15 | 16 kDa | 2 | 8 |
| fumigata) | | | | |
| Neutral/alkaline nonlysosomal ceramidase, putative - Aspergillus | B0XPL9 | 83 kDa | 0 | 13 |
| fumigatus A1163 | | | | |
| Fucose-specific lectin - Aspergillus fumigatus (Sartorya fumigata) | Q8NJT4 | 35 kDa | 4 | 11 |
| Aldose 1-epimerase, putative - Aspergillus fumigatus A1163 | B0XWH7 | 51 kDa | 5 | 0 |
| Mutanase - Aspergillus fumigatus A1163 | B0Y904 | 138 kDa | 0 | 15 |
| Amidase, putative - Aspergillus fumigatus A1163 | B0Y0P8 | 65 kDa | 13 | 0 |
| Alpha-N-acetylglucosaminidase, putative - Aspergillus fumigatus | B0XRY5 | 86 kDa | 12 | 0 |
| A1163 | | | | |
| Beta-lactamase, putative - Aspergillus fumigatus A1163 | B0Y2K9 | 48 kDa | 12 | 0 |
| Class V chitinase, putative - Aspergillus fumigatus A1163 | B0XXM2 | 44 kDa | 0 | 16 |
| Thioredoxin reductase GliT - Aspergillus fumigatus A1163 | B0Y818 | 36 kDa | 0 | 15 |
| Endo-1,3-beta-glucanase Engl1 - Neosartorya fischeri | A1D4K0_NEOFI | 78 kDa | 0 | 14 |
| Extracellular phytase, putative - Neosartorya fischeri | A1DJ51_NEOFI | 58 kDa | 14 | 0 |
| Cell wall glucanase - Aspergillus fumigatus A1163 | B0XUB8 | 47 kDa | 12 | 0 |
| Extracellular lipase, putative - Aspergillus fumigatus A1163 | B0YAB3 | 61 kDa | 0 | 11 |
| Tyrosinase, putative - Aspergillus fumigatus A1163 | B0XX11 | 42 kDa | 0 | 14 |
| Putative uncharacterized protein - Aspergillus fumigatus A1163 | B0XVQ9 | 20 kDa | 0 | 15 |
| Oxalate decarboxylase, putative - Aspergillus fumigatus (Sartorya | Q4X060 | 49 kDa | 11 | 0 |
| fumigata) | | | | |
| Alpha-L-rhamnosidase C, putative - Aspergillus fumigatus A1163 | B0Y024 | 88 kDa | 0 | 11 |
| Cellulase family protein - Aspergillus fumigatus A1163 | B0Y2L4 | 43 kDa | 4 | 5 |
| DUF1237 domain protein - Aspergillus fumigatus A1163 | B0XVA1 | 59 kDa | 6 | 5 |
| Asp-hemolysin precursor - Aspergillus fumigatus (Sartorya fumigata) | ASPH | 15 kDa | 13 | 0 |
| NlpC/P60-like cell-wall peptidase, putative - Aspergillus fumigatus | B0Y269 | 39 kDa | 9 | 0 |
| A1163 | | | | |
| Putative uncharacterized protein - Aspergillus fumigatus A1163 | B0Y742 | 79 kDa | 9 | 0 |
| ML domain protein - Neosartorya fischeri | A1DH49_NEOFI | 19 kDa | 0 | 8 |
| Metallopeptidase MepB - Aspergillus fumigatus A1163 | B0YB44 | 82 kDa | 0 | 5 |
| Putative uncharacterized protein - Aspergillus fumigatus A1163 | B0Y1A9 | 69 kDa | 0 | 9 |
| Alpha-amylase, putative - Neosartorya fischeri | A1CYB1_NEOFI | 69 kDa | 0 | 10 |
| Isoamyl alcohol oxidase, putative - Aspergillus fumigatus A1163 | B0YBR0 | 112 kDa | 4 | 2 |
| Isoamyl alcohol oxidase - Aspergillus fumigatus A1163 | B0XYQ0 | 66 kDa | 9 | 0 |
| Aldehyde dehydrogenase AldA, putative - Aspergillus fumigatus | B0Y8I3 | 61 kDa | 0 | 6 |
| A1163 | | | | |
| Mn superoxide dismutase - Aspergillus fumigatus A1163 | B0Y6Y9 | 25 kDa | 0 | 9 |
| Autophagic serine protease Alp2 - Neosartorya fischeri | A1DER5_NEOFI | 53 kDa | 0 | 10 |
| Cutinase, putative - Aspergillus fumigatus A1163 | B0XRY3 | 22 kDa | 0 | 9 |
| Glycosyl hydrolase, putative - Aspergillus fumigatus A1163 | B0Y4Y2 | 37 kDa | 10 | 0 |
| Gamma-glutamyltranspeptidase - Aspergillus fumigatus A1163 | B0YCI4 | 54 kDa | 9 | 0 |
| 1,3-beta-glucanosyltransferase Gel2 - Aspergillus fumigatus A1163 | B0Y8H9 | 52 kDa | 0 | 9 |
| Putative uncharacterized protein - Aspergillus fumigatus A1163 | B0XZT4 | 9 kDa | 0 | 10 |
| Ribonuclease T2, putative - Neosartorya fischeri | A1D1B0_NEOFI | 32 kDa | 0 | 3 |
| Extracellular cellulase CelA/allergen Asp F7-like, putative - | B0Y3V5 | 36 kDa | 6 | 0 |
| Aspergillus fumigatus A1163 | | | | |
| Oligopeptidase family protein - Aspergillus fumigatus A1163 | B0Y9Z2 | 80 kDa | 0 | 5 |
| 1,3-beta-glucanosyltransferase Gel3 - Aspergillus fumigatus A1163 | B0XT09 | 57 kDa | 8 | 0 |
| Xylosidase/arabinosidase, putative - Aspergillus fumigatus A1163 | B0XV35 | 41 kDa | 0 | 5 |
| Aspartic endopeptidase Pep2 - Aspergillus fumigatus A1163 | B0XXK9 | 43 kDa | 0 | 7 |
| Spermidine synthase - Neosartorya fischeri | A1D269_NEOFI | 33 kDa | 0 | 5 |
| NAD-dependent formate dehydrogenase AciA/Fdh - Aspergillus | B0YCV9 | 46 kDa | 0 | 3 |
| fumigatus A1163 | | | | |
| Elastinolytic metalloproteinase Mep - Aspergillus fumigatus A1163 | B0Y9E2 | 69 kDa | 0 | 7 |
| Beta-glucosidase, putative - Aspergillus fumigatus A1163 | B0XPE1 | 95 kDa | 6 | 0 |
| Alpha-mannosidase - Aspergillus fumigatus A1163 | B0XYH2 | 124 kDa | 0 | 4 |
| Peptidase, putative - Aspergillus fumigatus A1163 | B0Y766 | 46 kDa | 0 | 5 |
| Putative uncharacterized protein - Aspergillus fumigatus A1163 | B0Y1N8 | 22 kDa | 0 | 6 |
| Alpha-1,2-mannosidase, putative subfamily - Aspergillus fumigatus | B0XM55 | 87 kDa | 6 | 0 |
| A1163 | | | | |
| G-protein comlpex beta subunit CpcB - Aspergillus clavatus | A1CIN8_ASPCL | 35 kDa | 0 | 6 |
| Alpha-amylase, putative - Aspergillus fumigatus (Sartorya fumigata) | Q4WFV4 | 62 kDa | 0 | 5 |
| Nucleoside diphosphate kinase - Aspergillus fumigatus A1163 | B0Y2U5 | 17 kDa | 0 | 4 |
| Putative uncharacterized protein - Aspergillus fumigatus A1163 | B0Y1K3 | 37 kDa | 0 | 6 |
| Putative uncharacterized protein - Aspergillus fumigatus A1163 | B0Y1Z9 | 51 kDa | 4 | 0 |
| Phosphatidylglycerol specific phospholipase, putative - Aspergillus | B0XWP7 | 54 kDa | 0 | 4 |
| fumigatus A1163 | | | | |
| Class III chitinase ChiA1 - Aspergillus fumigatus A1163 | B0Y2Y2 | 87 kDa | 0 | 6 |
| Penicillolysin/deuterolysin metalloprotease, putative - Aspergillus | B0Y4X9 | 39 kDa | 0 | 5 |
| fumigatus A1163 | | | | |
| Beta-glucosidase, putative - Aspergillus fumigatus A1163 | B0XPB8 | 83 kDa | 3 | 0 |
| Feruloyl esterase, putative - Aspergillus fumigatus A1163 | B0Y7U1 | 58 kDa | 4 | 0 |
| Carboxypeptidase S1, putative - Aspergillus fumigatus A1163 | B0Y3N9 | 68 kDa | 5 | 0 |
| Cellulase, putative - Neosartorya fischeri | A1DGM6_NEOFI | 45 kDa | 5 | 0 |
| Aspartyl aminopeptidase - Aspergillus clavatus | A1CJU9_ASPCL | 54 kDa | 0 | 3 |
| Ser/Thr protein phosphatase family - Aspergillus fumigatus A1163 | B0XZB5 | 71 kDa | 0 | 2 |
| Putative uncharacterized protein - Aspergillus fumigatus A1163 | B0Y221 | 29 kDa | 0 | 3 |
| Putative uncharacterized protein - Aspergillus fumigatus A1163 | B0XT52 | 21 kDa | 0 | 3 |
| Putative uncharacterized protein - Neosartorya fischeri | A1DEN1_NEOFI | 32 kDa | 2 | 0 |
| Mannitol-1-phosphate dehydrogenase - Neosartorya fischeri | A1DGY9_NEOFI | 43 kDa | 0 | 4 |
| Beta-mannosidase - Aspergillus fumigatus A1163 | B0Y7S2 | 104 kDa | 4 | 0 |
| Alpha-1,2-mannosidase family protein - Aspergillus fumigatus | Q4WV22 | 89 kDa | 3 | 0 |
| (Sartorya fumigata) | | | | |
| Prolidase pepP, putative - Aspergillus fumigatus A1163 | B0XW47 | 52 kDa | 0 | 3 |
| Alpha-1,2-mannosidase, putative - Aspergillus fumigatus A1163 | B0YCI0 | 88 kDa | 3 | 0 |
| Cu,Zn superoxide dismutase SOD1 - Aspergillus fumigatus A1163 | B0Y476 | 16 kDa | 0 | 4 |
| Major allergen Asp f 2 precursor - Aspergillus fumigatus (Sartorya | ALL2 | 33 kDa | 0 | 3 |
| fumigata) | | | | |
| Pectate lyase A - Aspergillus fumigatus A1163 | B0XT32 | 34 kDa | 0 | 4 |
| Putative uncharacterized protein - Neosartorya fischeri | A1D462_NEOFI | 75 kDa | 4 | 0 |
| Lipase, putative - Aspergillus fumigatus A1163 | B0YCB0 | 49 kDa | 2 | 0 |
| BYS1 domain protein, putative - Aspergillus fumigatus A1163 | B0Y209 | 16 kDa | 0 | 3 |
| Carboxypeptidase CpyA/Prc1, putative - Neosartorya fischeri | A1DP75_NEOFI | 61 kDa | 0 | 3 |
| Endo-1,3(4)-beta-glucanase, putative - Aspergillus fumigatus A1163 | B0XM89 | 31 kDa | 0 | 2 |
| Acid phosphatase, putative - Aspergillus fumigatus A1163 | B0YF50 | 30 kDa | 2 | 0 |
| Extracellular lipase, putative - Aspergillus fumigatus A1163 | B0Y0A5 | 47 kDa | 3 | 0 |
| Putative uncharacterized protein - Aspergillus fumigatus A1163 | B0YCY3 | 23 kDa | 0 | 3 |
| Alpha-L-rhamnosidase B, putative - Aspergillus fumigatus A1163 | B0XPH6 | 75 kDa | 2 | 0 |
| Acid phosphatase AphA - Aspergillus fumigatus A1163 | B0Y1M6 | 67 kDa | 2 | 0 |
| GPI anchored protein, putative - Aspergillus fumigatus A1163 | B0XQH8 | 39 kDa | 2 | 0 |
| Aminotransferase, class V, putative - Aspergillus fumigatus A1163 | B0XQ69 | 42 kDa | 0 | 2 |
| Hydrolase, putative - Aspergillus fumigatus A1163 | B0XU31 | 72 kDa | 0 | 2 |
| Cell wall glucanase, putative - Aspergillus fumigatus A1163 | B0Y7N9 | 46 kDa | 3 | 0 |
| Putative uncharacterized protein - Aspergillus fumigatus A1163 | B0XYL8 | 23 kDa | 0 | 2 |
| Pectin methylesterase, putative - Aspergillus fumigatus A1163 | B0Y9F9 | 35 kDa | 2 | 0 |
| WSC domain protein, putative - Aspergillus fumigatus A1163 | B0Y4W9 | 31 kDa | 2 | 0 |
| Vacuolar aspartyl aminopeptidase Lap4, putative - Aspergillus | A1C4U0_ASPCL | 55 kDa | 0 | 2 |
| clavatus | (+4) | | | |
| Putative uncharacterized protein - Neosartorya fischeri | A1CYA5_NEOFI | 37 kDa | 0 | 2 |
| Pectin lyase, putative - Aspergillus fumigatus A1163 | B0Y0N7 | 40 kDa | 2 | 0 |
| GPI anchored protein, putative - Aspergillus fumigatus A1163 | B0Y935 | 44 kDa | 0 | 2 |
| Dihydrolipoyl dehydrogenase - Neosartorya fischeri | A1CYQ9_NEOFI | 55 kDa | 0 | 2 |
| Putative uncharacterized protein - Aspergillus fumigatus A1163 | B0YDZ8 | 12 kDa | 0 | 2 |
| N,O-diacetyl muramidase, putative - Aspergillus fumigatus A1163 | B0Y858 | 25 kDa | 2 | 0 |
| Conidial hydrophobin RodB - Neosartorya fischeri | A1D142_NEOFI | 14 kDa | 0 | 2 |
| Hydroxyacylglutathione hydrolase, putative - Neosartorya fischeri | A1DDQ5_NEOFI | 28 kDa | 0 | 2 |

TABLE 4
All theoretical and detected weight of peptides released after
AfuS28 and SedB digestion of NPY1-36 and 3-36 by MS.

| Peptides generated from NPY degradation and | | Theoritical | | |
|---|---|---|---|---|
| detected on MS | z charge | mass | Mass detected | Delta |
| SKPDNPGEDAPAEDMARYYSALRHYINLITRQRY (NPY3-36) | 3 | 1337.9915 | 1337.9955 | −0.004 |
| SEQ ID NO: 27 | | | | |
| | 4 | 1003.7454 | 1003.7503 | −0.0049 |
| | 5 | 803.1978 | 803.2006 | −0.0028 |
| | 6 | 669.4994 | 669.5016 | −0.0022 |
| | 7 | 574.0005 | 574.0027 | −0.0022 |
| DNPGEDAPAEDMARYYSALRHYINLITRQRY (NPY6-36) | 4 | 925.7005 | 925.7052 | −0.0047 |
| SEQ ID NO: 29 | | | | |
| | 5 | 740.7619 | 740.765 | −0.0031 |
| | 6 | 617.4694 | 617.4723 | −0.0029 |
| GEDAPAEDMARYYSALRHYINLITRQRY (NPY9-36) | 3 | 1125.224 | 1125.2304 | −0.0064 |
| SEQ ID NO: 30 | | | | |
| | 4 | 844.1699 | 844.1734 | −0.0035 |
| | 5 | 675.5373 | 675.5398 | −0.0025 |
| | 6 | 563.1157 | 563.1184 | −0.0027 |
| AEDMARYYSALRHYINLITRQRY (NPY14-36) | 3 | 968.8304 | 968.8364 | −0.006 |
| SEQ ID NO: 31 | | | | |
| | 4 | 726.8746 | 726.8778 | −0.0032 |
| | 5 | 581.7012 | 581.7042 | −0.003 |
| | 6 | 484.9188 | 484.9216 | −0.0028 |
| YPSKPDNPGEDAPAEDMARYYSALRHYINLITRQRY (NPY1-36) | 3 | 1424.6969 | 1424.7073 | −0.0104 |
| SEQ ID NO: 26 | | | | |
| | 4 | 1068.7745 | 1068.7804 | −0.0059 |
| | 5 | 855.221 | 855.2258 | −0.0048 |
| | 6 | 712.8521 | 712.8568 | −0.0047 |
| | 7 | 611.164 | 611.16 | 0.004 |
| YPSKPDNP (SEQ ID NO: 32) | 1 | 917.4363 | 917.4372 | −0.0009 |
| | 2 | 459.2218 | 459.2228 | −0.001 |
| Peptides generated from 3-36 NPY degradation | | Theoritical | | |
| and detected on MS | z charge | mass | Mass detected | Delta |
| YPS | 1 | 366.166 | 366.1676 | −0.0016 |
| KPD | 1 | 359.1925 | 359.1941 | −0.0016 |
| NPG | 1 | 287.135 | 287.1363 | −0.0013 |
| SKP | 1 | 331.1976 | 331.1985 | −0.0009 |
| DNP | 1 | 345.1405 | 345.1411 | −0.0006 |
| SKPDNP (SEQ ID NO: 33) | 1 | 657.3202 | 657.3216 | −0.0014 |
| GED | 1 | 320.1088 | 320.1096 | −0.0008 |
| GEDAP (SEQ ID NO: 34) | 1 | 488.1987 | 488.1995 | −0.0008 |
| AP | 1 | 187.1077 | 187.108 | −0.0003 |
| APA | 1 | 258.1448 | 258.1453 | −0.0005 |
| EDM | 1 | 394.1279 | 394.1288 | −0.0009 |
| ARY | 2 | 205.1133 | 205.1137 | −0.0004 |
| YSA | 1 | 340.1503 | 340.151 | −0.0007 |
| LRH | 1 | 425.2619 | 425.2628 | −0.0009 |
| YIN | 1 | 409.2082 | 409.2107 | −0.0025 |
| LIT | 1 | 346.2336 | 346.2343 | −0.0007 |
| RQR | 1 | 459.2786 | 459.2797 | −0.0011 |
| Y | 1 | 182.0812 | 182.0814 | −0.0002 |
| AED | 1 | 334.1245 | 334.1256 | −0.0011 |
| MAR | 1 | 377.1966 | 377.1973 | −0.0007 |
| YYS | 1 | 432.1765 | 432.1775 | −0.001 |
| ALR | 1 | 359.2401 | 359.2405 | −0.0004 |
| HYI | 1 | 432.2241 | 432.225 | −0.0009 |
| NLI | 1 | 359.2289 | 359.2302 | −0.0013 |
| TRQ | 1 | 404.2252 | 404.2261 | −0.0009 |
| RY | 1 | 338.1823 | 338.1829 | −0.0006 |

Characterization of Recombinant A. fumigatus Prolylexoprotease

Aspergillus fumigatus Pep1 (Reichard et al., 1995), SedB (Reichard et al., 2006) and SedC secreted at pH 3.5 as well as Alp1, Mep (Sarfati et al., 2006), DppIV (Beauvais et al., 1997a, 1997b), Lap1 and Lap2 (Monod et al., 2005) secreted at pH 7.0 were previously characterized as recombinant enzymes. To learn more about the function of the new serine protease AfuS28 and its importance in protein digestion, the enzyme was produced as a recombinant protein with or without a His₆-tail using P. pastoris as an expression system. A yield of 25 μg/ml culture supernatant was obtained. Fractions of the purified enzyme showed a single band on silver stained SDS-PAGE gel, attesting a high degree of purity (Data not shown). AfuS28 is a 65 kDa glycoprotein with a carbohydrate content of about 20% (FIG. 3). Recombinant AfuS28 had the same electrophoretic mobility than the native enzyme secreted by A. fumigatus in collagen medium.

Recombinant AfuS28 showed no detectable proteolytic activity using casein resorufin-labeled as a substrate but very efficiently released pNA when AAP-pNA, APP-pNA and AAAP-pNA were used as substrates. AfuS28 was active between pH 3.0 and 9.0 with an optimum at pH 6.0. At optimal pH, AfuS28 activity was 1.5, 1 and 0.2 mmol min⁻¹ me (specific activity) using AAAP-pNA, AAP-pNA and APP-pNA respectively. AfuS28 showed no activity on the DppIV substrates GP-pNA and AP-pNA, and on APF-pNA which is a SedB substrate.

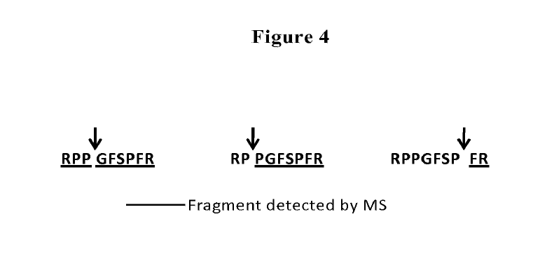

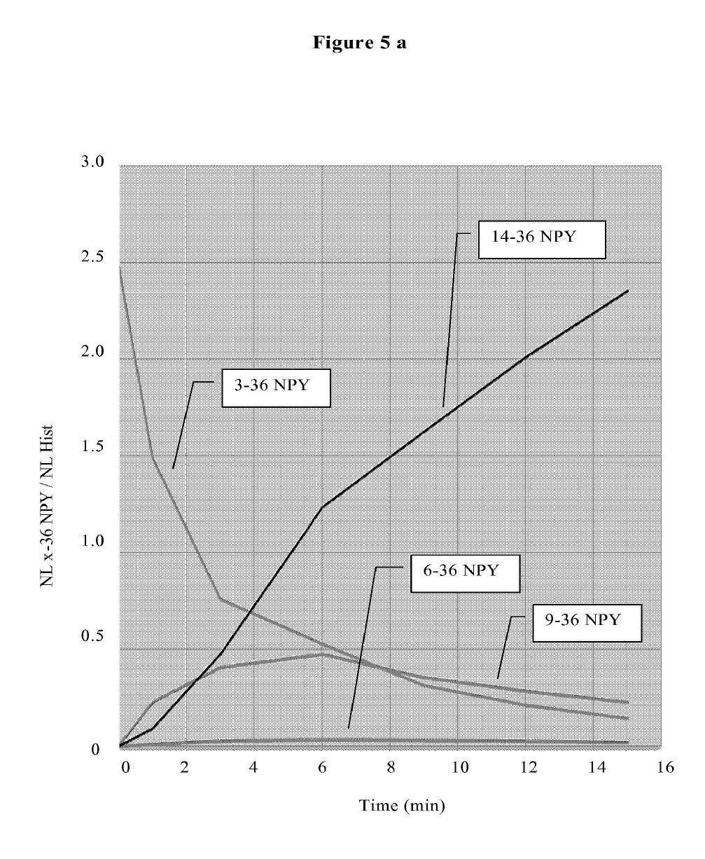

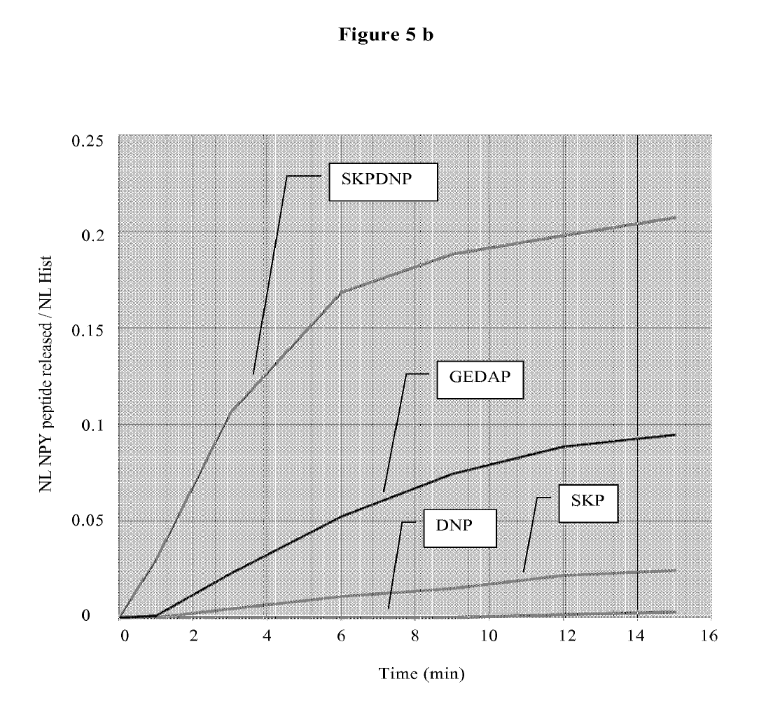

The degradation of large proline-containing peptides by AfuS28 was analyzed by mass spectrometry. Digestion of bradykinin (RPPGFSPFR, SEQ ID NO:28) resulted in RPP, FR, GFSPFR (SEQ ID NO:35) and PGFSPFR (SEQ ID NO:36) fragments (FIG. 4, RPPGFSP fragment is SEQ ID NO:37). AfuS28 was also found to degrade NPY1-36 and NPY3-36, cleaving after proline residues (FIG. 6). Measurements of the degradation kinetics of the amide form of NPY3-36 were accomplished. The reaction was monitored at different times from 0 to 15 min (t1, t3, t6, t9, t12 and t15 min) using 1.8 mU of enzyme at 37° C. (FIG. 5). The signal of histidine (m/z 156.0766, present from the elution of AfuS28 Hist₆-Tag) was used as reference to normalize peptide fragment intensities (Table 4), permitting to follow the progression of NPY3-36 degradation. At t₀, intact NPY3-36 was observed at different m/z ratios corresponding to various charge states (z=3-7). Less than 10% of full length NPY3-36 was still present after 15 min incubation. Fragments NPY6-36, NPY9-36 and NPY14-36 were detected concomitantly with SKP (residues 3-5), SKPDNP (residues 3-8) and GEDAP (residues 9-13) after 3 min of digestion by AfuS28. NPY6-36 and DNP (residues 6-8) were detected only in low amount as SKPDNP appeared to be highly resistant to AfuS28. At t₁ (1 min) there was more NPY9-36 than NPY14-36 and following 6 min, NPY14-36 increased at the expense of NPY9-36. The latter disappeared after 60 min reaction (Data not shown). Importantly, a peak corresponding to fragment NPY3-13 (SKPDNPGEDAP) was never detected. Therefore, cleavage between amino acids P8 and G9 seemed necessary for cleavage after P in position 13. These results are in agreement with AfuS28 having an exoprotease activity.

Large Peptide Digestion into Short Assimilable Peptides at Acidic pH.

Large peptide digestion at acidic pH was investigated with SedB, the major exoprotease secreted at acidic pH by A. fumigatus. NPY3-36 was not digested by recombinant SedB, but this enzyme removed tripeptides (YPS, KPD and NPG) from the N-terminus of NPY1-36 until position 10 (FIG. 6.b). In conclusion, SedB appeared to be active only when the amino acid in P1 or P′1 position (amino acids in positions 3 and 4 from the N-terminus of any substrate peptide) was not a proline. AfuS28 and SedB added together degraded NPY3-36 in Y, di- and tri-peptides (FIG. 6.a). Two different pathways of degradation can be hypothesized. In the first one, SedB cleaves NPY9-36 generated by AfuS28 in tripeptides and jumpes P13 which does not constitute a road block. In the second, AfuS28 first acts on P13 before further SedB digestion. Other tripeptides such as INL, ITR or QRY which would result from other modes of degradation were not detected.

### Degradation of Gliadin

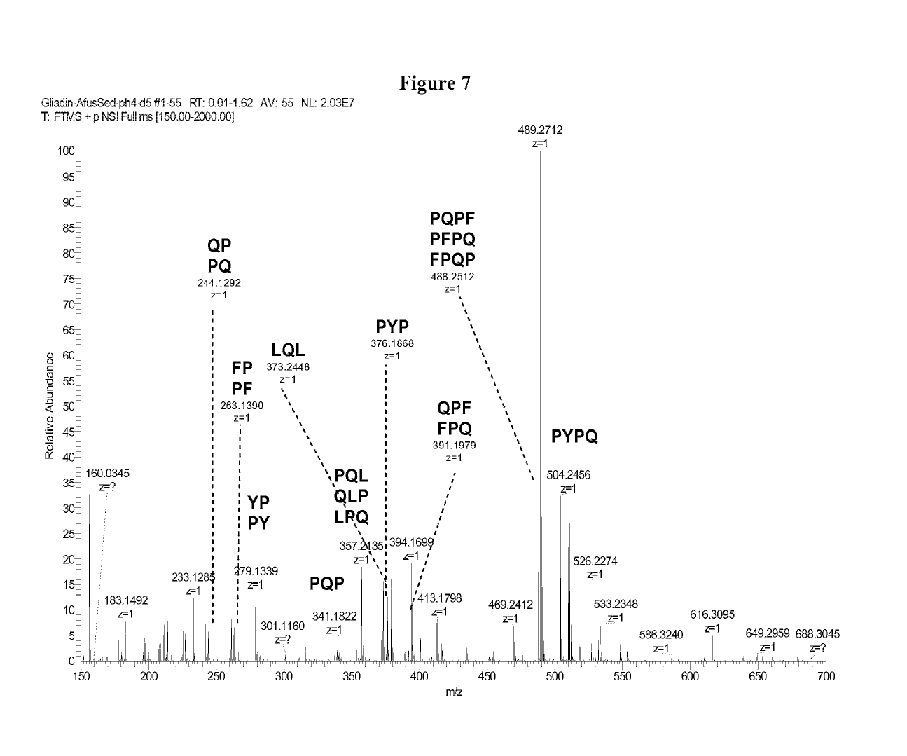

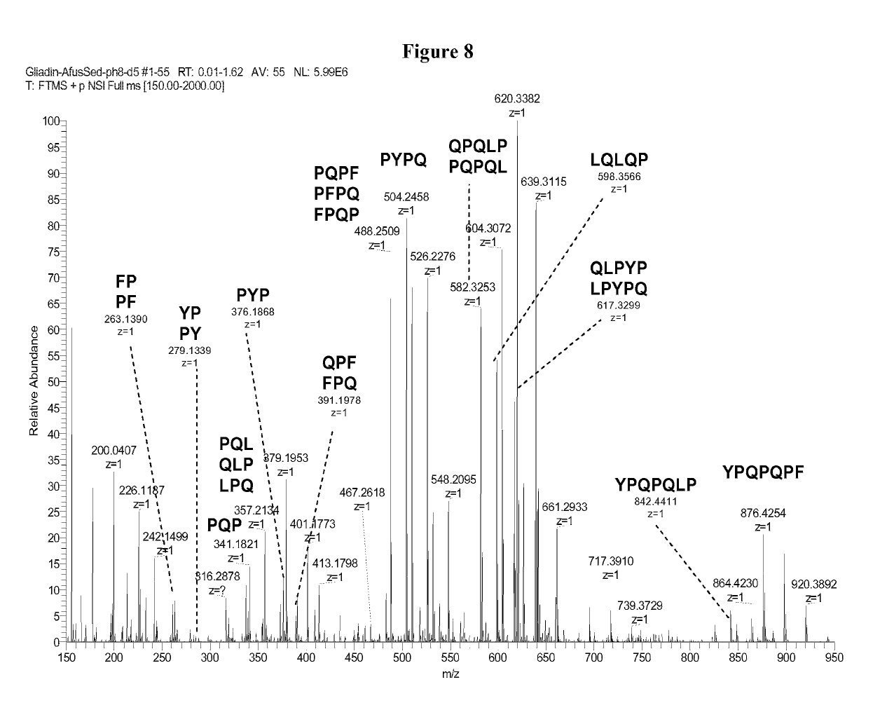

The 33-mer of gliadin (5 nmol) was incubated at 37° C. for 2 hours at pH 4 and pH 8 in the presence of 1 μg of AfuS28 and SedB in a total volume of 45 μl of buffer (the ratio substrate/enzyme was 1/20). The used buffers were those disclosed by Michel Monod in J. Proteome Res, 2010. The enzyme activity was stopped with 5 μl of formic acid 0.5%. The samples (total volume ˜50 μl) were then diluted 5 times in H₂O:MeCN 50:50 (+0.1% formic acid) and infused in the LTQ-Orbitrap via Nanomate (150-2000 m/z, 1.5 min). The enzyme composition AfuS28+SedB provides complete degradation of gliadin into several di-, tri-, tetra- and pentapeptides (FIGS. 7 and 8)

1. An enzyme composition, comprising
i. a prolyl protease AfuS28 comprising SEQ ID NO: 1, a biologically active fragment thereof, a naturally occurring allelic variant thereof, or a sequence having at least 95% of identity, and
ii. at least one tripeptidyl protease of the sedolisin family, said tripeptidyl protease is selected from the group consisting of
a) a sedolisin SedA comprising SEQ ID NO: 2, a biologically active fragment thereof, a naturally occurring allelic variant thereof, or a sequence having at least 95% of identity,
b) a sedolisin SedB comprising SEQ ID NO: 3, a biologically active fragment thereof, a naturally occurring allelic variant thereof, or a sequence having at least 95% of identity,
c) a sedolisin SedC comprising SEQ ID NO: 4, a biologically active fragment thereof, a naturally occurring allelic variant thereof, or a sequence having at least 95% of identity, and
d) a sedolisin SedD comprising SEQ ID NO: 5, a biologically active fragment thereof, a naturally occurring allelic variant thereof, or a sequence having at least 95% of identity.

2. The enzyme composition of claim 1, comprising
a prolyl protease AfuS28 comprising SEQ ID NO: 1, a biologically active fragment thereof, a naturally occurring allelic variant thereof, or a sequence having at least 95% of identity, and
a sedolisin SedB comprising SEQ ID NO: 3, a biologically active fragment thereof, a naturally occurring allelic variant thereof, or a sequence having at least 95% of identity.

3. The enzyme composition of claim 1, comprising
i) a prolyl protease AfuS28 comprising SEQ ID NO: 1, a biologically active fragment thereof, a naturally occurring allelic variant thereof, or a sequence having at least 95% of identity,
ii) a sedolisin SedA comprising SEQ ID NO: 2, a biologically active fragment thereof, a naturally occurring allelic variant thereof, or a sequence having at least 95% of identity,
iii) a sedolisin SedB comprising SEQ ID NO: 3, a biologically active fragment thereof, a naturally occurring allelic variant thereof, or a sequence having at least 95% of identity,
iv) a sedolisin SedC comprising SEQ ID NO: 4, a biologically active fragment thereof, a naturally occurring allelic variant thereof, or a sequence having at least 95% of identity, and
v) a sedolisin SedD comprising SEQ ID NO: 5, a biologically active fragment thereof, a naturally occurring allelic variant thereof, or a sequence having at least 95% of identity.

4. The enzyme composition according to claim 1, further comprising at least one protease selected from the group consisting of:
an aspartic protease of the pepsin family (Pep1) comprising SEQ ID NO: 6, a biologically active fragment thereof, a naturally occurring allelic variant thereof, or a sequence having at least 95% of identity,
a glutamic protease serine comprising SEQ ID NO: 7, a biologically active fragment thereof, a naturally occurring allelic variant thereof, or a sequence having at least 95% of identity,
carboxypeptidase Scp1 comprising SEQ ID NO:8, a biologically active fragment thereof, a naturally occurring allelic variant thereof, or a sequence having at least 95% of identity, and
X-prolyl peptidase (DppIV) comprising SEQ ID NO:9, a biologically active fragment thereof, a naturally occurring allelic variant thereof, or a sequence having at least 95% of identity.

5. A pharmaceutical composition, comprising the enzyme composition of any one of claims 1 to 4 and at least one pharmaceutically acceptable excipient, carrier and/or diluent.

6. The pharmaceutical composition of claim 5, wherein said pharmaceutical composition is an oral pharmaceutical composition.

7. A food supplement comprising the enzyme composition of any one of claims 1 to 4.

8. A method for treating and/or preventing a syndrome associated with a human disease or disorder, said disease or disorder being selected from the group consisting of celiac disease, digestive tract bad absorption, an allergic reaction, an enzyme deficiency, a fungal infection, mycoses, Crohn disease, and sprue, the method comprising administering to a subject in need thereof a therapeutically effective amount of the enzyme composition of any one of claims 1 to 4.

9. The method according to claim 8, wherein the allergic reaction is a reaction to gluten or fragments thereof.

10. The method according to claim 9, wherein a fragment of gluten is gliadine.

11. (canceled)

12. (canceled)

13. A method of degrading a polypeptide substrate, said method comprising contacting the polypeptide substrate with the enzyme composition of any one of claims 1 to 4.

14. The method of degrading a polypeptide substrate according to claim 13, wherein said enzyme composition sequentially digests a full-length polypeptide substrate or a full-length protein.

15. The method of degrading a polypeptide substrate according to claim 13, wherein the polypeptide substrate is casein, gluten, bovine serum albumin or fragments thereof.

16. The method of degrading a polypeptide substrate according to claim 13, wherein the polypeptide substrate length is from 2 to 200 amino acids.

17. A method of detoxifying gliadin, the method comprising contacting a gliadin containing food product with an effective dose of the enzyme composition of any one of claims 1 to 4.

18. A method for improving food digestion in a mammal, the method comprising orally administering to the mammal the enzyme composition of any one of claims 1 to 4.

19. The method for improving food digestion according to claim 18, wherein the food contains proline rich nutriments.

20. The method for improving food digestion according to claim 18, wherein the mammal is a human.

21. A kit for degrading a polypeptide product comprising the enzyme composition of any one of claims 1 to 4.

22. A method for producing the enzyme composition of any one of claims 1 to 4, the method comprising
(a) introducing into a host cell a nucleic acid encoding for
i. a prolyl protease AfuS28 comprising SEQ ID NO: 1, a biologically active fragment thereof, a naturally occurring allelic variant thereof, or a sequence having at least 95% of identity, and
ii. at least one tripeptidyl protease of the sedolisin family, said tripeptidyl protease selected from the group consisting of
a) a sedolisin SedA comprising SEQ ID NO: 2, a biologically active fragment thereof, a naturally occurring allelic variant thereof, or a sequence having at least 95% of identity,
b) a sedolisin SedB comprising SEQ ID NO: 3, a biologically active fragment thereof, a naturally occurring allelic variant thereof, or a sequence having at least 95% of identity,
c) a sedolisin SedC comprising SEQ ID NO: 4, a biologically active fragment thereof, a naturally occurring allelic variant thereof, or a sequence having at least 95% of identity, and
d) a sedolisin SedD comprising SEQ ID NO: 5, a biologically active fragment thereof, a naturally occurring allelic variant thereof, or a sequence having at least 95% of identity;
(b) cultivating the cell of step (a) in a culture medium under conditions suitable for producing the enzyme composition; and
(c) recovering the enzyme composition.

23. The method for producing the enzyme composition according to claim 22, wherein the nucleic acid encoding for X-prolyl peptidase (DppIV) comprising SEQ ID NO:9, a biologically active fragment thereof, a naturally occurring allelic variant thereof, or a sequence having at least 95% of identity is introduced into the host cell.

24. The method for producing the enzyme composition according to claim 22, wherein the host cell is Pichia pastoris, Aspergillus oryzae, Saccharomyces cerevisiae, and/or Kluveromyces lactis.

25. The method according to claim 13, wherein the polypeptide substrate is selected from the group consisting of a by-product; a toxic or contaminant protein; a prion or virus; a protein used in proteomics; and a cornified substrate.

26. The method according to claim 13, wherein the degrading of a polypeptide substrate is used for wound cleaning; for hydrolysing a polypeptide for amino acid analysis; for a cosmetology procedure; for prothesis cleaning and/or preparation; for use in fabric softeners; for use in soaps; for tenderizing meat; for the controlled fermentation process of Soja or cheese; for cleaning or disinfection of septic tanks or any container containing proteins that should be removed or sterilized; or for cleaning of surgical instruments.

27. The method according to claim 26, wherein the cosmetology procedure is selected from the group consisting of a cosmetology procedure involving a peeling tool, depilation, dermabrasion and dermaplaning.

28. The method according to claim 19, wherein the proline rich nutriment is gluten.
